(19) United States
(12) Patent Application Publication
(10) Pub. No.: US 2024/0229156 A1
(43) Pub. Date: Jul. 11, 2024

(54) METHODS FOR USING A HYPOMETHYLATING AGENT TO TREAT DISEASES AND DISORDERS BASED ON GENE MUTATION PROFILES

(71) Applicant: Celgene Corporation, Summit, NJ (US)

(72) Inventors: Daniel Elvyn LOPES DE MENEZES, Berkeley, CA (US); Wendy Laurene SEE, Novato, CA (US); Alberto RISUEÑO PÉREZ, Gelves (ES); Anjan Guha THAKURTA, Basking Ridge, NJ (US); Charles Lee BEACH, Less Summit, MO (US); Ignazia LA TORRE, Neuchatel (CH); Barry Sim SKIKNE, Leawood, KS (US); Manuel UGIDOS GUERRERO, Cádiz (ES)

(21) Appl. No.: 18/550,083

(22) PCT Filed: Mar. 11, 2022
(86) PCT No.: PCT/US2022/019874

§ 371 Date: Sep. 11, 2023

Related U.S. Application Data
(60) Provisional application No. 63/160,671, filed on Mar. 12, 2021.

(51) Int. Cl. C12Q 1/6886; C12Q 1/6869

(52) U.S. Cl. CPC C12Q 1/6886; C12Q 1/6869; C12Q 2600/106; C12Q 2600/154; C12Q 2600/156

(57) ABSTRACT

Provided herein are methods of using a hypomethylating agent (e.g., 5-azacytidine or decitabine), optionally in combination with one or more additional therapeutic agents or therapies, to treat diseases and disorders including cancers such as but not limited to acute myeloid leukemia (AML), and myelodysplastic syndromes (MDS), based on gene mutation profiles of the diseases and disorders.

### CROSS-REFERENCE TO RELATED APPLICATIONS

This application claims priority to U.S. Provisional Application No. 63/160,671, filed on Mar. 12, 2021, the entirety of which is incorporated herein by reference.

### FIELD

Provided herein are methods of using a hypomethylating agent (e.g., 5-azacytidine or decitabine), optionally in combination with one or more additional therapeutic agents or therapies, to treat diseases and disorders including cancers such as but not limited to acute myeloid leukemia (AML) and myelodysplastic syndromes (MDS), based on gene mutation profiles of the diseases and disorders.

### BACKGROUND

Cancer is a major worldwide public health problem, and is among the leading cause of deaths worldwide. Many types of cancer have been described in the medical literature. Examples include cancer of the blood, bone, lung (e.g., non-small-cell lung cancer and small-cell lung cancer), colon, breast, prostate, ovary, brain, and intestine. According to the National Cancer Institute, the most common cancers (listed in descending order according to estimated new cases in 2020) are breast cancer, lung and bronchus cancer, prostate cancer, colon and rectum cancer, melanoma of the skin, bladder cancer, non-Hodgkin lymphoma, kidney and renal pelvis cancer, endometrial cancer, leukemia, pancreatic cancer, thyroid cancer, and liver cancer. (https://www.cancer.gov/about-cancer/understanding/statistics). The incidence of cancer continues to climb as the general population ages and as new forms of cancer develop. According to the American Cancer Society, one in three people will be diagnosed with cancer in their lifetime. A continuing need exists for effective therapies to treat cancer patients.

Myelodysplastic syndromes (MDS) refers to a diverse group of hematopoietic stem cell disorders. The annual incidence of MDS is estimated to be 4.9 cases per 100,000 people worldwide, and approximately 10,000 people in the United States are diagnosed with MDS each year. MDS may be characterized by a cellular marrow with impaired morphology and maturation (dysmyelopoiesis), peripheral blood cytopenias, and a variable risk of progression to acute leukemia, resulting from ineffective blood cell production.

MDS are grouped together because of the presence of dysplastic changes in one or more of the hematopoietic lineages including dysplastic changes in the myeloid, erythroid, and megakaryocytic series. These changes result in cytopenias in one or more of the three lineages. Patients afflicted with MDS may develop complications related to anemia, neutropenia (infections), and/or thrombocytopenia (bleeding). About 10% to about 70% of patients with MDS may develop acute leukemia. In the early stages of MDS, the main cause of cytopenias is increased programmed cell death (apoptosis). As the disease progresses and converts into leukemia, a proliferation of leukemic cells overwhelms the healthy marrow. The disease course differs, with some cases behaving as an indolent disease and others behaving aggressively with a very short clinical course that converts into an acute form of leukemia. The majority of people with higher risk MDS eventually experience bone marrow failure. Up to 50% of MDS patients succumb to complications, such as infection or bleeding, before progressing to AML.

Acute myeloid leukemia (AML) is a type of cancer that affects the bone marrow and blood. AML is known by a variety of names, including acute myelogenous leukemia, acute myeloblastic leukemia, acute granulocytic leukemia, and acute nonlymphocytytic leukemia. The word “acute” in acute myeloid leukemia reflects the disease's rapid progression. It is called acute myeloid leukemia because it affects a group of white blood cells called the myeloid cells, which normally develops into the various types of mature blood cells, such as red blood cells, white blood cells, and platelets. In other words, AML is a malignancy of the myeloid precursor cell line, characterized by the rapid proliferation of abnormal cells, which accumulate in the bone marrow and interfere with the production of normal cells.

AML is generally classified as de novo, or secondary when arising following exposure to prior cytotoxic chemotherapy, or after a history of prior myelodysplastic syndrome (MDS) or antecedent hematologic disorder (AHD). The pathogenesis of AML at the genetic level is also heterogeneous. Genetic alterations that cause AML include an internal tandem duplication in a tyrosine kinase gene, chromosomal rearrangements that alter the functioning of genes involved in leukemogenesis, and mutations resulting in activation of transcription factors, etc. Comprehensive profiling of genetic alterations in AML will enhance disease classification, risk stratification and prognosis, and ultimately, allow more precise therapeutic interventions. MV4-11 and MOLM-13 are AML cell lines that express FLT3 mutations. See Quentmeier et al., Leukemia, 17(1):120-4 (January 2003). FLT3-ITD up-regulates MCL-1 to promote survival of stem cells in AML. See Yoshimoto et al., Blood, 114(24):5034-43 (Dec. 3, 2009).

Current strategies of AML treatment include inductive chemotherapy for remission induction and low-intensity therapy intended for survival prolongation. The remission-induction chemotherapy is a cytoreductive modality for achieving remission or at least effective reduction of tumor burden. The combination of cytarabine and anthracycline has been the mainstay of treatments to induce remission. A common induction regimen consists of cytarabine at doses of 100 to 200 mg/m²/day for 7 days and daunorubicin at doses of 45 to 90 mg/m²/day for 3 days, often referred to as the “7+3 protocol.” If remission is achieved, additional cycles of chemotherapy or stem cell transplantation from a donor (allogeneic hematopoietic stem cell transplantation [HSCT]) are employed for consolidation. Although inductive chemotherapy has become the standard for younger fit patients, it remains a matter of debate in the elderly and unfit population. In elderly patients who have received IC, outcomes are less favorable primarily due to the increased rate of treatment-related death and poor prognostic factors leading to lower remission rates seen in the elderly population. Treatment options for patients considered ineligible or unfit due to age, performance status, and co-morbidities or those who choose not to receive IC current chemotherapy options include low-dose cytarabine, 5-azacytidine or decitabine.

Although induction chemotherapy produces morphologic complete remissions (CRs) in about 60% to 80% of younger adults and 40% to 50% of older adults with newly diagnosed AML, there is a substantial population of patients who will fail to attain CR (i.e., refractory AML). Even for those who attain CR after induction treatment, a significant portion will eventually relapse, leading to only about 29% relapse-free survival at three years after treatment.

Thus, there is a need for more effective treatments for treating cancer, including but not limited to AML, and/or MDS, and this disclosure addresses this need.

### SUMMARY

Provided herein are methods of using hypomethylating agent (e.g., 5-azacytidine or decitabine), optionally in combination with one or more additional therapeutic agents or therapies, to treat diseases and disorders including cancers such as but not limited to acute myeloid leukemia (AML), and myelodysplastic syndromes (MDS), based on gene mutation profiles of the diseases and disorders, for example, the presence or absence of certain mutant alleles.

In one embodiment, provided herein is a method of treating a cancer or a disorder related to abnormal cell proliferation in a human subject, comprising administering to the human subject a therapeutically effective amount of a hypomethylating agent, and wherein the cancer or the disorder is characterized by:
(i) presence of a nucleophosmin (NPM1) mutation;
(ii) presence of a NPM1 mutation and a FMS-like tyrosine kinase-3 (FLT3) mutation;
(iii) presence of a DNA methyltransferase 3A (DNMT3A) mutation;
(iv) presence of a tumor protein p53 (TP53) mutation; or
(v) presence of an isocitrate dehydrogenase 2 (IDH2) mutation and a serine-and-arginine-rich-splicing-factor 2 (SRSF2) mutation.

In one embodiment, provided herein is a method of treating a cancer or a disorder related to abnormal cell proliferation in a human subject, comprising:
(1) identifying the cancer or the disorder in the human subject to be characterized by:
(i) presence of a nucleophosmin (NPM1) mutation;
(ii) presence of a NPM1 mutation and a FMS-like tyrosine kinase-3 (FLT3) mutation;
(iii) presence of a DNA (cytosine-5)-methyltransferase 3A (DNMT3A) mutation;
(iv) presence of a tumor protein p53 (TP53) mutation; or
(v) presence of an isocitrate dehydrogenase 2 (IDH2) mutation and a serine-and-arginine-rich-splicing-factor 2 (SRSF2) mutation; and
(2) administering to the human subject a therapeutically effective amount of a hypomethylating agent.

In one embodiment, the method further comprises administering at least one additional therapeutic agent or therapy. In one embodiment, the at least one additional therapeutic agent or therapy is an agent that modulates NPM1 pathway or targets nucleophosmin, a FLT3 inhibitor, an agent or therapy that restore the wild-type DNMT3A function, a PI3K/Akt/mTOR pathway inhibitor, a TP53 inhibitor, an agent that targets spliceosome or its downstream target(s), an IDH2 inhibitor, or a RAS pathway inhibitor.

In one embodiment, the hypomethylating agent is 5-azacytidine. Certain embodiments herein provide that the 5-azacytidine is administered as a composition that is a single unit dosage form. Certain embodiments herein provide compositions that are non-enteric-coated. Certain embodiments herein provide compositions that are immediate release oral compositions.

### BRIEF DESCRIPTION OF THE DRAWINGS

FIG. 1A shows overall survival (OS) for patients in the defined AML subtype and treated with either placebo or CC-486. FIG. 1B shows relapse-free survival (RFS) for patients in the defined AML subtype and treated with either placebo or CC-486.

FIG. 2 represents cytogenetic risk (intermediate- or poor-risk) at diagnosis; favorable-risk patients were excluded from the trial.

FIG. 3A shows OS for patients with intermediate cytogenetic risk treated with either CC-486 or placebo. FIG. 3B shows RFS for patients with intermediate cytogenetic risk treated with either CC-486 or placebo. FIG. 3C shows OS for patients with poor cytogenetic risk treated with either CC-486 or placebo. FIG. 3D shows RFS for patients with poor cytogenetic risk treated with either CC-486 or placebo.

FIG. 4A to FIG. 4C represent prevalence of gene mutations at diagnosis and associated hazard ratios for RFS comparing CC-486 versus placebo.

FIG. 5A shows OS for patients with NPM1 mutation versus NPM1 wild type treated with CC-486. FIG. 5B shows OS for patients with NPM1 mutations versus NPM1 wild type treated with placebo. FIG. 5C shows RFS for patients with NPM1 mutations versus NPM1 wild type treated with CC-486. FIG. 5D shows RFS for patients with NPM1 mutation versus NPM1 wild type treated with placebo.

FIG. 6A shows overall survival (OS) for patients harboring NPM1 mutation or NPM1 wild type and treated with either placebo or CC-486. FIG. 6B shows relapse-free survival (RFS) for patients harboring NPM1 mutation or NPM1 wild type and treated with either placebo or CC-486. FIGS. 6C and 6D shows overall survival (OS) analysis and hazard ratios comparing NPM1 mutation status in combination with MRD status in patients treated with CC-486 or placebo. FIGS. 6E and 6F shows relapse-free survival (RFS) analysis and hazard ratios comparing NPM1 mutation status in combination with MRD status in patients treated with CC-486 or placebo.

In FIG. 7A, OS hazard ratio for NPM1 mutation versus NPM1 wild type in CC-486 treated patients was visualized using a forest plot (left) and reported in the table (right) along with 95% confidence interval [CI] and associated p-value. In FIG. 7B, OS hazard ratio for NPM1 mutation versus NPM1 wild type in placebo treated patients was visualized using a forest plot (left) and reported in the table (right) along with 95% confidence interval (CI) and associated p-value. In FIG. 7C, RFS hazard ratio for NPM1 mutation versus NPM1 wild type in CC-486 treated patients was visualized using a forest plot (left) and reported in the table (right) along with 95% confidence interval (CI) and associated p-value. In FIG. 7D, RFS hazard ratio for NPM1 mutation versus NPM1 wild type in placebo treated patients was visualized using a forest plot (left) and reported in the table (right) along with 95% confidence interval (CI) and associated p-value.

In FIG. 8A, OS hazard for patients with NPM1 mutations in CC-486 versus placebo was visualized using a forest plot (left) and reported in the table (right) along with 95% CI and associated p-value. In FIG. 8B, RFS hazard ratio for patients with NPM1 mutations in CC-486 versus placebo was visualized using a forest plot (left) and reported in the table (right) along with 95% CI and associated p-value.

FIG. 9A shows overall survival (OS) for patients harboring FLTITD/FLT3-TKD mutation or FLTITD/FLT3-TKD wild type and treated with either placebo or CC-486. FIG. 9B shows RFS for patients harboring FLTITD/FLT3-TKD mutation or FLTITD/FLT3-TKD wild type and treated with either placebo or CC-486.

FIG. 10A and FIG. 10B show OS and RFS for patients harboring NPM1 mutation and FLTITD wild type and treated with either placebo or CC-486. FIG. 10C and FIG. 10D show OS and RFS for patients harboring NPM1 mutation and FLTITD mutation and treated with either placebo or CC-486. FIG. 10E and FIG. 10F show OS and RFS for patients harboring NPM1 wild type and FLTITD mutation and treated with either placebo or CC-486.

In FIG. 11A, OS hazard ratio for NPM1 mutation FLTITD WT, NPM1 mutation FLTITD mutation, and NPM1 WT FLTITD mutation and (versus patients that lack the reported NPM1 mutation status in combination with FLTITD mutation status) in CC-486 treated patients was visualized using a forest plot (left) and reported in the table (right) along with 95% confidence interval [CI] and associated p-value. In FIG. 11B, OS hazard ratio for NPM1 mutation FLTITD WT, NPM1 mutation FLTITD mutation, and NPM1 WT FLTITD mutation (versus patients that lack the reported NPM1 mutation status in combination with FLTITD mutation status) in placebo treated patients was visualized using a forest plot (left) and reported in the table (right) along with 95% confidence interval (CI) and associated p-value. In FIG. 11C, RFS hazard ratio for NPM1 mutation FLTITD WT, NPM1 mutation FLTITD mutation, and NPM1 WT FLTITD mutation (versus patients that lack the reported NPM1 mutation status in combination with FLTITD mutation status) in CC-486 treated patients was visualized using a forest plot (left) and reported in the table (right) along with 95% confidence interval (CI) and associated p-value. In FIG. 11D, RFS hazard ratio for NPM1 mutation FLTITD WT, NPM1 mutation FLTITD mutation, and NPM1 WT FLTITD mutation (versus patients that lack the reported NPM1 mutation status in combination with FLTITD mutation status) in placebo treated patients was visualized using a forest plot (left) and reported in the table (right) along with 95% confidence interval (CI) and associated p-value.

In FIG. 12A, OS hazard for patients with NPM1 mutation FLTITD WT, NPM1 mutation FLTITD mutation, and NPM1 WT FLTITD mutation in CC-486 versus placebo was visualized using a forest plot (left) and reported in the table (right) along with 95% CI and associated p-value. In FIG. 12B, RFS hazard ratio for patients with NPM1 mutation FLTITD WT, NPM1 mutation FLTITD mutation, and NPM1 WT FLTITD mutation in CC-486 versus placebo was visualized using a forest plot (left) and reported in the table (right) along with 95% CI and associated p-value.

FIG. 13A and FIG. 13B show OS for patients harboring NPM1 mutation FLTITD/FLT3-TKD wild type versus NPM1 mutation FLTITD/FLT3-TKD mutation or FLTITD/FLT3-TKD mutation NPM1 mutation versus FLTITD/FLT3-TKD mutation NPM1 WT and treated with either placebo or CC-486. FIG. 13C and FIG. 13D show RFS for patients harboring NPM1 mutation FLTITD/FLT3-TKD wild type versus NPM1 mutation FLTITD/FLT3-TKD mutation or FLTITD-FLT3-TKD mutation NPM1 mutation versus FLTITD/FLT3-TKD mutation NPM1 WT and treated with either placebo or CC-486.

FIG. 14A and FIG. 14B represent gene mutation frequency and mutational landscape in patients (median age of 68 years) who achieved complete response CR or CRi, post-induction chemotherapy/consolidation (postIC/C). FIG. 14C shows the variant allele frequencies (VAFs) of gene mutations included in the analysis.

FIG. 15 represents gene mutation status (postIC/C, in CR or CRi) and associated hazard ratios with clinical response in patients treated with CC-486 versus placebo, visualized using a forest plot (left) and reported in the table (right) along with 95% CI and associated p-value.

FIG. 16A and FIG. 16B show OS for patients with a normal karyotype (no abnormalities), abnormal karyotype (1-3 abnormalities), or complex karyotype (>4 abnormalities) treated with either CC-486 or placebo. FIG. 16C and FIG. 16D show RFS for patients with a normal karyotype (no abnormalities), abnormal karyotype (1-3 abnormalities), or complex karyotype (>4 abnormalities) treated with either CC-486 or placebo.

FIG. 17A and FIG. 17B show OS and RFS for patients with a normal karyotype treated with either CC-486 or placebo. FIG. 17C and FIG. 17D show OS and RFS for patients with an abnormal karyotype (>1 abnormality) treated with either CC-486 or placebo.

FIG. 18A and FIG. 18B show OS and RFS for patients with DNMT3A mutation or WT and treated with either CC-486 or placebo. FIG. 18C and FIG. 18D show OS and RFS for patients with p53 mutation or WT and treated with either CC-486 or placebo.

In FIG. 19A and FIG. 19B, RFS hazard ratios for mutations in functional categories are reported for patients treated with CC-486 or placebo. In FIG. 19C and FIG. 19D, RFS hazard ratios for mutations in functional categories for patients treated with CC-486 versus placebo were visualized using a forest plot and reported in the table along with 95% confidence interval (CI) and associated p-value.

FIG. 20A and FIG. 20B represent statistical associations of co-occurring gene mutations in patients that achieved CR or CRi, postIC/C. The commutation frequencies are listed in the Table (FIG. 20A). FIG. 20B is a heat map showing the odds ratio for all possible pairwise comparisons between individual gene mutations, karyotypes, chromosomal abnormalities, and fusion genes.

FIG. 21A and FIG. 21B show the clinical associations (RFS) in patients with mutations in SRSF2 alone or SRSF2 WT, or IDH2 alone or IDH2 WT and treated with CC-486 versus placebo. FIG. 21C and FIG. 21D show RFS curves for co-occurrence of SRS2 and IDH2 versus SRSF/IDH2 WT (other) with CC-486 treatment or placebo.

### DETAILED DESCRIPTION

### I. Definitions

Unless defined otherwise, all technical and scientific terms used herein have the same meaning as commonly understood by one of ordinary skill in the art. All publications and patents referred to herein are incorporated by reference herein in their entireties.

As used in the specification and the accompanying claims, the indefinite articles “a” and “an” and the definite article “the” include plural as well as singular referents, unless the context clearly dictates otherwise.

The term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 30%, 25%, 20%, 15%, 10%, 9%, 8%, 7%, 6%, 5%, 4%, 3%, 2%, 1%, 0.5%, 0.1%, or 0.05% of a given value or range.

As used herein, and unless otherwise specified, the terms “treat,” “treating” and “treatment” refer to the eradication or amelioration of a disease or disorder, or of one or more symptoms associated with the disease or disorder. In certain embodiments, the terms refer to minimizing the spread or worsening of the disease or disorder resulting from the administration of one or more prophylactic or therapeutic agents to a subject with such a disease or disorder. In some embodiments, the terms refer to the administration of a compound or dosage form provided herein, with or without one or more additional active agent(s), after the onset of symptoms of the particular disease.

As used herein, and unless otherwise specified, the terms “prevent,” “preventing” and “prevention” refer to the prevention of the onset, recurrence or spread of a disease or disorder, or of one or more symptoms thereof. In certain embodiments, the terms refer to the treatment with or administration of a compound or dosage form provided herein, with or without one or more other additional active agent(s), prior to the onset of symptoms, particularly to subjects at risk of disease or disorders provided herein. The terms encompass the inhibition or reduction of a symptom of the particular disease. Subjects with familial history of a disease in particular are candidates for preventive regimens in certain embodiments. In addition, subjects who have a history of recurring symptoms are also potential candidates for prevention. In this regard, the term “prevention” may be interchangeably used with the term “prophylactic treatment.”

As used herein, and unless otherwise specified, the terms “manage,” “managing” and “management” refer to preventing or slowing the progression, spread or worsening of a disease or disorder, or of one or more symptoms thereof. Often, the beneficial effects that a subject derives from a prophylactic and/or therapeutic agent do not result in a cure of the disease or disorder. In this regard, the term “managing” encompasses treating a subject who had suffered from the particular disease in an attempt to prevent or minimize the recurrence of the disease.

As used herein, amelioration of the symptoms of a particular disorder by administration of a particular pharmaceutical composition refers to any lessening, whether permanent or temporary, lasting or transient, that can be attributed to or associated with administration of the composition.

As used herein, and unless otherwise specified, the terms “therapeutically effective amount” and “effective amount” of a compound mean an amount sufficient to provide a therapeutic benefit in the treatment or management of a disease or disorder, or to delay or minimize one or more symptoms associated with the disease or disorder. A “therapeutically effective amount” and “effective amount” of a compound mean an amount of therapeutic agent, alone or in combination with one or more other agent(s), which provides a therapeutic benefit in the treatment or management of the disease or disorder. The terms “therapeutically effective amount” and “effective amount” can encompass an amount that improves overall therapy, reduces or avoids symptoms or causes of disease or disorder, or enhances the therapeutic efficacy of another therapeutic agent.

As used herein, and unless otherwise specified, a “prophylactically effective amount” of a compound is an amount sufficient to prevent a disease or disorder, or prevent its recurrence. A prophylactically effective amount of a compound means an amount of therapeutic agent, alone or in combination with one or more other agent(s), which provides a prophylactic benefit in the prevention of the disease. The term “prophylactically effective amount” can encompass an amount that improves overall prophylaxis or enhances the prophylactic efficacy of another prophylactic agent.

“Tumor,” as used herein, refers to all neoplastic cell growth and proliferation, whether malignant or benign, and all pre-cancerous and cancerous cells and tissues. “Neoplastic,” as used herein, refers to any form of dysregulated or unregulated cell growth, whether malignant or benign, resulting in abnormal tissue growth. Thus, “neoplastic cells” include malignant and benign cells having dysregulated or unregulated cell growth.

The terms “cancer” and “cancerous” refer to or describe the physiological condition in mammals that is typically characterized by unregulated cell growth. Examples of cancer include, but are not limited to blood borne (e.g., lymphoma, leukemia) and solid tumors.

The terms “composition,” “formulation,” and “dosage form,” as used herein are intended to encompass compositions comprising the specified ingredient(s) (in the specified amounts, if indicated), as well as any product(s) which result, directly or indirectly, from combination of the specified ingredient(s) in the specified amount(s). By “pharmaceutical” or “pharmaceutically acceptable” it is meant that any diluent(s), excipient(s) or carrier(s) in the composition, formulation, or dosage form are compatible with the other ingredient(s) and not deleterious to the recipient thereof. Unless indicated otherwise, the terms “composition,” “formulation,” and “dosage form” are used herein interchangeably.

The term “immediate release,” when used herein in reference to a composition, formulation, or dosage form provided herein, means that the composition, formulation, or dosage form does not comprise a component (e.g., a coating) that serves to delay the spatial and/or temporal release of some or all of the API from the composition, formulation, or dosage form beyond the stomach following oral administration. In certain embodiments, an immediate release composition, formulation, or dosage form is one that releases the API substantially in the stomach following oral administration. In specific embodiments, an immediate release composition, formulation, or dosage form is one that is not delayed-release. In specific embodiments, an immediate release composition, formulation, or dosage form is one that does not comprise an enteric coating.

The term “non-enteric-coated,” when used herein, refers to a pharmaceutical composition, formulation, or dosage form that does not comprise a coating intended to release the active ingredient(s) beyond the stomach (e.g., in the intestine). In certain embodiments, a non-enteric-coated composition, formulation, or dosage form is designed to release the active ingredient(s) substantially in the stomach.

The term “substantially in the stomach,” when used herein in reference to a composition, formulation, or dosage form provided herein, means that at least about 99%, at least about 95%, at least about 90%, at least about 85%, at least about 80%, at least about 75%, at least about 70%, at least about 65%, at least about 60%, at least about 55%, at least about 50%, at least about 45%, at least about 40%, at least about 35%, at least about 30%, at least about 25%, at least about 20%, at least about 15%, or at least about 10% of the active ingredient (e.g., 5-azacytidine) is released in the stomach. The term “released in the stomach” and related terms as used herein refer to the process whereby the active ingredient (e.g., 5-azacytidine) is made available for uptake by or transport across cells lining the stomach and then made available to the body.

The term “subject” is defined herein to include animals such as mammals, including, but not limited to, primates (e.g., humans), cows, sheep, goats, horses, dogs, cats, rabbits, rats, mice and the like. In specific embodiments, the subject is a human.

The terms “co-administration” and “in combination with” include the administration of two or more therapeutic agents either simultaneously, concurrently or sequentially within no specific time limits. In one embodiment, the agents are present in the cell or in the subject's body at the same time or exert their biological or therapeutic effect at the same time. In one embodiment, the therapeutic agents are in the same composition or unit dosage form. In other embodiments, the therapeutic agents are in separate compositions or unit dosage forms. In certain embodiments, a first agent can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapeutic agent.

As used herein, the terms “determining,” “detecting,” “measuring,” “identifying” and “evaluating,” as used herein generally refer to any form of measurement, and include determining whether an element is present or not. These terms include quantitative and/or qualitative determinations. Assessing may be relative or absolute. “Detecting the presence of” can include determining the amount of something present, as well as determining whether it is present or absent.

As used herein, the term “responsive” or “responsiveness” when used in reference to a treatment refers to the degree of effectiveness of the treatment in lessening or decreasing the symptoms of a disease, e.g. AML or MDS, being treated. However, it is also understood that responsiveness can also halt disease progression, and does not necessarily require lessening or decreasing the symptoms of the disease.

The term “sample” as used herein relates to a material or mixture of materials, typically, although not necessarily, in fluid form, containing one or more components of interest. An exemplary sample is a “biological sample” obtained from a biological subject, including a sample of biological tissue or fluid origin, obtained, reached, or collected in vivo or in situ. A biological sample also includes samples from a region of a biological subject containing pre-cancerous or cancer cells or tissues. Such samples can be, but are not limited to, organs, tissues, and cells isolated from a mammal. Exemplary biological samples include but are not limited to cell lysate, a cell culture, a cell line, a tissue, oral tissue, gastrointestinal tissue, an organ, an organelle, a biological fluid, a blood sample, a urine sample, a skin sample, and the like. Preferred biological samples include, but are not limited to, whole blood, partially purified blood, PBMC, tissue biopsies, including bone marrow core biopsy, bone marrow aspirate, isolated bone marrow mononuclear cells, circulating tumor cells and the like.

As used herein, the term “hypomethylating agent” means a compound that inhibits DNA methylation, for example when administered to a subject, such as a subject with MDS or AML. As used herein and unless otherwise specified, a hypomethylating agent also includes a DNA methyltransferase (DNMT) inhibitor that leads to inhibition of DNA methylation. Examples of hypomethylating agents for use in treating MDS or AML include decitabine (5-aza-2′-deoxycytidine) and azacytidine (5-azacytidine). While not being bound by theory, hypomethylating agents are believed to inhibit the activity of methyltransferase, causing hypomethylation of DNA which prevents normal DNA synthesis and results in subsequent cytotoxicity.

As used herein, the term “minimal residual disease (MRD)” refers to the small number of cancer cells that remain in the body after treatment. The number of remaining cells may be so small that they do not cause any physical signs or symptoms and often cannot even be detected through traditional methods, such as viewing cells under a microscope and/or by tracking abnormal serum proteins in the blood. An MRD positive test result (i.e., “positive MRD”) means that residual (remaining) disease was detected. An MRD negative result (i.e., “negative MRD”) means that minimal residual disease was not detected.

### II. Overview

The present disclosure is based in part on the discovery that the gene mutational landscape is significantly altered in patients at diagnosis versus screening (post IC/C). Different specific gene mutation profiles favor hypomethylating agent (e.g., 5-azacytidine) response over placebo treatment at diagnosis versus post IC/C. For example, at diagnosis, NPM1 mutations are prognostic while FLT3/FLTITD mutations are adverse; both of these mutation subgroups gain significantly prolonged benefit with 5-azacytidine compared to placebo. Surprisingly, patients with poor prognostic mutations in FLT3/FLTITD in combination with NPM1 mutations gain similar OS benefit and prolonged RFS benefit with 5-azacytidine compared to patients with NPM1 mutations and wild type FLT3/FLTITD. In the post IC/C setting, DNMT3A mutations are adverse and patients with these mutations gain significant benefit with 5-azacytidine; 5-azacytidine benefits were also observed in patients with mutations in TP53 or SRSF2; RAS pathway alterations or SRSF2/IDH2 co-mutations confer adverse risk; and patients with DNA methylation or transcription gene mutations significantly benefit with 5-azacytidine post IC/C.

In another aspect, the discovery is of combination treatment of a hypomethylating agent (e.g., 5-azacytidine) and other agents or therapies based on the gene mutation profiles. Without being bound by a particular theory, the additional agents or therapies can be those that target the gene profiles that benefit hypomethylating agent (e.g., 5-azacytidine) treatment, to further improve the benefits of hypomethylating agent (e.g., 5-azacytidine) treatment. Without being bound by a particular theory, the additional agents or therapies can also be those that target the gene profiles that confer adverse risk to hypomethylating agent (e.g., 5-azacytidine) treatment, to complement hypomethylating agent (e.g., 5-azacytidine) treatment.

In some embodiments, certain combinations work synergistically in the treatment of particular diseases or disorders, including, e.g., types of cancer and certain diseases and conditions associated with, or characterized by, undesired angiogenesis or abnormal cell proliferation.

Acute myeloid leukemia (AML), also known as acute myelogenous leukemia, is an aggressive, heterogeneous, myeloid malignancy. According to the American Cancer Society, AML is the most common type of leukemia diagnosed in adults and makes up 32% of all adult leukemia cases. It is estimated that approximately 19,940 people will be diagnosed with AML in 2020 in the United States with 11,180 patients estimated to die from the disease. The disease is particularly difficult to treat in older adults who account for the majority of patients; thus, the five-year overall survival is only approximately 29%. National Cancer Institute, SEER Cancer Stat Facts: Leukemia—Acute Myeloid Leukemia (AML), https://seer.cancer.gov/statfacts/html/amyl.html (accessed 10 Jun. 2020). Since the 1970s, initial standard therapy, for those fit enough to receive it, consisted of the ‘7+3’ regimen, which includes seven days of continuous infusion cytarabine and three days of an anthracycline. Rai et. al. Blood 1981:58: 1203-1212. Over the next 35 years, a number of clinical trials attempting to augment the safety and efficacy of AML treatment have been performed with little change in the standard of care. However, recent data detailing the molecular ontogeny of AML have elucidated causal pathways which have led to efforts to develop targeted drug therapies. E. Winer and R. Stone, Ther. Adv. Hematol., 10:PMC6624910 (July 2019).

There is a long felt need for the gene mutation profile-based treatments, including combination treatments, described herein, as AML has a high rate of relapse. Additionally, relapsed and refractory AML is a disease that is very difficult to treat and is likely driven by multiple abnormal signaling pathways that give the leukemic cell an advantage in overcoming any single pathway that is being inhibited. Thus, successful target treatments or combination treatments are highly desirable to treat AML patients.

In one aspect of the methods of treatment described herein, the patient to be treated is about age 60 or older. In another aspect of the methods of treatment described herein, the patient to be treated is about age 65 or older, about age 70 or older, about age 75 or older, or about age 80 or older. In yet another aspect, the patient is a relapsed AML patient. In another aspect, the patient is a refractory AML patient. The patient to be treated can also be under about age 60, under about age 55, under about age 50, under about age 45, or under about age 40. In other aspects, the patient to be treated has FLT3 mutations, either FLT3-ITD or FLT3-TKD. In some aspects, the patient to be treated has a recurrent AML mutation. Exemplary AML mutations include, but are not limited to, FMS-related tyrosine kinase 3 (FLT3), Kirsten rat sarcoma viral oncogene homolog (KRAS), neuroblastoma RAS viral (V-Ras) oncogene homolog (NRAS), proto-oncogene c-Kit (KIT), protein tyrosine phosphatase non-receptor type 11 (PTPN11), neurofibromin 1 (NF1), DNA methyltransferase 3A (DNMT3A), isocitrate dehydrogenase 1 (IDH1), isocitrate dehydrogenase 2 (IDH2), ten-eleven translocation-2 (TET2), additional sex comb-like 1 (ASXL1), enhancer of zeste homolog 2 (EZH2), mixed-lineage leukemia 1/histone-lysine N-methyltransferase 2A (MLL/KMT2A), nucleophosmin (NPM1), CCAAT enhancer binding protein alpha (CEBPA), runt-related transcription factor 1 (RUNX1), GATA-binding factor 2 (GATA2), tumor protein p53 (TP53), serine-and-arginine-rich-splicing-factor 2 (SRSF2), U2 small nuclear RNA auxiliary factor 1 (U2AF1), splicing factor 3b subunit 1 (SF3B1), zinc finger (CCCH type), RNA-binding motif and serine/arginine rich 2 (ZRSR2), RAD21 cohesin complex component (RAD21), stromal antigen 1 (STAG1), stromal antigen 2 (STAG2), structural maintenance of chromosomes 1A (SMC1A), and structural maintenance of chromosomes protein 3 (SMC3). Various mutations are described in more detail herein.

NPM1: Nucleophosmin (NPM1) encodes a nucleus-cytoplasm shuttling protein and is involved in the regulation of several cellular processes that modulate growth-suppressive pathways, including ribosome biogenesis, genomic stability, p53-dependent stress response and modulation of growth-suppressive pathways via interaction with the Arf (Heath et al., Leukemia 2017, 31(4):798-807). Mutations in NPM1 occur in 25%-30% of AML patients (Papaemmanuil et al., N. Engl. J. Med. 2016, 374(23):2209-2221). The most common NPM mutations occur in exon 12 and >55 unique mutations have been reported in the literature (Falini et al., N. Engl. J. Med. 2005, 352(3):254-66; Heath 2017). NPM1 mutation type A, which results in an insertion of TCTG (thymine, cytosine, thymine, guanine) between nucleotides 860 and 863 (c.863_864insTCTG), is the most frequently found mutation in AML (Alpermann et al., Haematologica 2016, 101(2), e55-e58). Mutations in NPM1 lead to loss of the nucleolar localization signal and relocalization within the cytoplasm (Panuzzo et al., J. Clin. Med. 2020, 9(3):802).

FLT3: Fms-related tyrosine kinase 3, encodes a type III receptor tyrosine kinase FLT3. Mutations of the FLT3 gene occur in approximately 30% of all AML cases, with the internal tandem duplication (ITD) representing the most common type of FLT3 mutation and the point mutations in FLT3 TKD (D835) occur in approximately 7% of AML cases (Daver et al., Leukemia. 2019, 33(2):299-312; Papaemmanuil 2016). The receptor FLT3 is expressed in cell lineages that include hematopoietic stem and early progenitor cells and is involved in cell differentiation and proliferation (Rosnet et al., Leukemia 1996, 10:238-248). In AML, FLT3 is over-expressed on a majority of leukemic blast cell population in the bone marrow, as well as, FLT3-ITD mutations, have been reported to occur in the juxtamembrane domain of the receptor and point mutations in the tyrosine kinase domain (TKD, commonly at amino acid residue D835) (Yokota et al., Leukemia 1997, 11:1605-1609; Yamamoto et al., Blood 2001, 97:2434-2439; Kiyoi et al., Oncogene 2002, 21:2555-2563; Papaemmanuil 2016). FLT3-ITD and TKD mutations constitutive activate FLT3 signaling pathways which include, STAT5, AKT/mTOR, and Ras/MAPK (Hayakawa et al., Oncogene 2000, 19:624-631).

FLT3-ITD is associated with poor prognosis and is a considered a driver mutation of AML. FLT3-ITD repeat mutations are typically present in approximately 25% of AML cases, and the point mutations in FLT3 TKD occur in approximately 7% of AML cases. FLT3 mutations appear to predispose patients to poorer responses to induction therapy, shorter durations of remission, lower rates of overall survival and higher rates of relapse (Thiede et al., Blood 2002, 99(12):4326-35).

In the AML NCCN (Tallman et al., Acute Myeloid Leukemia, Version 3.2019, NCCN Clinical Practice Guidelines in Oncology, J. Natl. Compr. Canc. Netw. 2019, 17(6):721-749; O'Donnell et al., NCCN Clinical Practice Guidelines Acute myeloid leukemia, J. Natl. Compr. Canc. Netw. 2012, 10(8):984-1021) and ELN guidelines, the characterization of NPM1 mutations with or without FLT3ITD in patients are utilized to ascribe AML risk categories (Dohner et al., Blood 2017, 129(4):424-447; Dohner et al., Blood 2020, 135(5):371-380).

DNMT3A: The de novo DNA methyl transferase 3A (DNMT3A) gene encodes a protein involved in epigenetic regulation; it regulates gene expression through methylation of CpG islands. Mutations in DNMT3A are found in 15%-30% of patients with de novo AML (Papaemmanuil 2016). Most DNMT3A mutations are heterozygous and the common alteration occurs at amino acid R882H within the catalytic domain, resulting in a dominant negative consequence and is associated with reduced methytransferase activity (Ley et al., N. Engl. J. Med 2010, 363(25):2424-33; Russler-Germain et al., Cancer Cell 2014, 25(4):442-454). DNMT3A mutations are typically characterized as a CHIP mutation and is considered an early mutational event in leukemic transformation and appears to confer poor prognosis in AML patients (Brunetti et al., Cold Spring Harb. Perspect. Med 2017, 7(2):a030320; Ley et al., N. Engl. J. Med 2013, 368(22):2059-74).

TP53: Tumor suppressor protein 53 (TP53, P53) is a key transcription factor that regulates cell cycle arrest, apoptosis, senescence and DNA repair (Zhang et al., Leuk. Lymphoma 2017, 58(8):1777-1790). Mutations in TP53 occur in 5%-20% of AML patients. Mutations mostly occur in the DNA binding domain and the most common hot spot mutations are prevalent in codons 175, 245, 248, 249, 273, and 282 of the DNA binding domain (Papaemmanuil 2016; Welch, Best Pract. Res. Clin. Haematol. 2018, 31(4):379-383). The TP53 gene is located on chromosome 17p13.1, and chromosomal deletions of 17p (or monosomy chr17 or other chr17 abnormalities) phenocopies TP53 loss of function and portends poor survival in AML.

IDH2: Isocitrate dehydrogenase 2 is a metabolic enzyme that performs oxidative decarboxylation of isocitrate in ketoglutarate (KG), an irreversible reaction of the tricarboxylic acid cycle (TCA). Recurring mutations in isocitrate dehydrogenase 2 (IDH2) may be important for AML pathogenesis or disease progression (Mardis et al., N. Engl. J. Med 2009, 361(11):1058-66; Ley 2013; Papaemmanuil 2016). Mutations occur in ˜15% of AML patients and involve residues R140 or R172 that result in neomorphic activity (DiNardo et al., Am. J. Hematol. 2015, 90(8):732-736; Mardis, 2009; Ley 2013; Papaemmanuil 2016). Mutated IDH2 produces the oncometabaolite D-2-hydroxyglutarate (2-HG), which inhibits ten-eleven translocation (TET) family of proteins leading to increased DNA methylation. The prognositic significance of IDH2 mutations remains to be established and different disease contexts may be determinants in various AML disease segments (DiNardo 2015).

SRSF2: Serine and Arginine Rich Splicing Factor 2 (SRSF2) belongs to a family of pre-mRNA splicing components that regulate pre-mRNA splicing, RNA stability, and translation (Visconte et al., Cancers (Basel) 2019, 11(12):1844). In AML, SRSF2 mutations have been found in about 25% of patients and mutations occur almost exclusively at proline 95 and alter binding affinity of the RRM motif (Visconte 2019). In general, mutations in splicing factor genes predict worse prognosis in de novo AML patients (Hou et al., Oncotarget. 2016, 7(8):9084-101).

Hematopoietic stem cells (HSCs) are defined by their self-renewal capacity and ability to differentiate into the various blood lineage cell types, myeloid and lymphoid cell lineages (Pang et al., Proc. Natl. Acad Sci. U.S.A. 2011, 108(50):20012-7; Birbrair et al., Ann. N.Y. Acad Sci. 2016, 1370(1):82-96).

The function of HSCs gradually changes during aging. CHIP gene mutations are linked with clonal hematopoiesis are often associated with age, where patients/subjects carrying these mutations (e.g., DNMT3A, TET2, ASXL-1) present increased risk of developing AML and MDS (Papaemmanuil et al., Blood 2013, 122(22):3616-27; Cancer Genome Research Atlas Network, N. Engl. J. Med 2013, 368, 2059-2074; Ley 2013; Papaemmanuil 2016; Xie et al., Nat. Med 2014, 20(12):1472-8; Young et al., Nat. Commun. 2016, 7:12484).

In large sequencing studies, clonal hematopoiesis gene mutations were detected in normal aging subjects, and mutations frequencies increased with age (Jaiswal et al., Science 2019, 366:6465; Genovese et al., N. Engl. J. Med 2014, 371(26):2477-87; Shlush, Blood 2018, 131(5):496-504; Busque et al., Nat. Genet. 2012, 44:1179-1181).

In the Genovese et al. report, the CHIP mutation frequency was lower in younger subjects of less than 50 years of age (1%), whereas CHIP mutations were detected in ˜10% subjects over 65 years of age.

The frequently mutated genes include DNMT3A, TET2, and ASXL1, and are functionally involved in epigenetic regulation (DNA methylation (DNMT3A), DNA hydroxymethylation (TET2), and histone methylation and histone ubiquitination (ASXL1).

These are also important genetic features in AML and MDS and occur early during the malignant transformation process. These mutations were also observed in the study population described in this application.

CC-486 is a DNMT (DNA methyl transferase) inhibitor and hypomethylating agent, and the data provided herein provides evidence to show that CC-486 will benefit patient segments with DNMT3A and pathways where DNA methylation is altered (FIG. 19A to FIG. 19D, i.e., pathways altered). This data shows that administration of CC-486 and other DNMT inhibitors and/or hypomethylating agents will also benefit disease indications where similar mutations are pertinent.

Beyond myeloid diseases (AML, MDS), indications in cancers include, but are not limited to, primary gastrointestinal diffuse large B-cell lymphoma (Zhao et al., Oncol. Lett. 2015, 9(5):2307-2312); T cell lymphoma (Couronne et al., N. Engl. J. Med. 2012, 366(1):95-6); CHIP mutations confer adverse risk in multiple myeloma undergoing transplant (Mouhieddine et al., Nat. Commun. 2020, 11(1):2996); and solid tumors (Coombs et al., Cell Stem Cell 2017, 21(3):374-382; Comen et al., J. Natl. Cancer Inst. 2020, 112(1):107-110).

In another aspect, provided herein are methods of treating a human having myelodysplastic syndrome (MDS) by administering to the human (i) a pharmaceutical composition comprising a hypomethylating agent (e.g., 5-azacytidine or decitabine) administered orally; and (ii) at least one additional therapeutic agent. Also disclosed herein are (i) pharmaceutical compositions comprising a hypomethylating agent (e.g., 5-azacytidine or decitabine) administered orally, and (ii) at least one additional therapeutic agent for treating MDS in a human.

In some embodiments, the MDS is a higher risk or high risk MDS. Higher-risk MDS for this disclosure is defined as High or Very High risk according to the Revised International Scoring System (IPSS-R) (Voso M. T., et. al., J Clin Oncol. 2013; 31(21): 2671-2677; and Greenberg P. L. et al., Blood. 2012, 120(12): 2454-2465), where these patients have median survival of 1.6 and 0.8 years respectively.

### III. Method of Use

Provided herein are methods for treating a cancer or a disorder related to abnormal cell proliferation in a human subject.

In one embodiment, provided herein is a method of treating a cancer or a disorder related to abnormal cell proliferation in a human subject, comprising administering to the human subject a therapeutically effective amount of a hypomethylating agent, and wherein the cancer or the disorder is characterized by:
(i) presence of a nucleophosmin (NPM1) mutation;
(ii) presence of a NPM1 mutation and a FMS-like tyrosine kinase-3 (FLT3) mutation;
(iii) presence of a DNA methyltransferase 3A (DNMT3A) mutation;
(iv) presence of a tumor protein p53 (TP53) mutation; or
(v) presence of an isocitrate dehydrogenase 2 (IDH2) mutation and a serine-and-arginine-rich-splicing-factor 2 (SRSF2) mutation.

In one embodiment, provided herein is a method of treating a cancer or a disorder related to abnormal cell proliferation in a human subject, comprising:
(1) identifying the cancer or the disorder in the human subject to be characterized by:
(i) presence of a nucleophosmin (NPM1) mutation;
(ii) presence of a NPM1 mutation and a FMS-like tyrosine kinase-3 (FLT3) mutation;
(iii) presence of a DNA (cytosine-5)-methyltransferase 3A (DNMT3A) mutation;
(iv) presence of a tumor protein p53 (TP53) mutation; or
(v) presence of an isocitrate dehydrogenase 2 (IDH2) mutation and a serine-and-arginine-rich-splicing-factor 2 (SRSF2) mutation; and
(2) administering to the human subject a therapeutically effective amount of a hypomethylating agent.

In one embodiment, the cancer or the disorder is characterized by the presence of a NPM1 mutation.

In one embodiment, the cancer or the disorder is characterized by the presence of a NPM1 mutation and a FLT3 mutation. In one embodiment, the FLT3 mutation is a FMS-like tyrosine kinase-3 internal tandem duplication (FLT3-ITD) or FMS-like tyrosine kinase-3 tyrosine kinase domain (FLT3-TKD) mutation. In one embodiment, the FLT3 mutation is a FLT3-ITD mutation. In one embodiment, the FLT3 mutation is a FLT3-TKD mutation. In one embodiment, the FLT3 mutation is a FLT3-ITD and FLT3-TKD co-mutation.

In one embodiment, the cancer or the disorder is characterized by the presence of a DNMT3A mutation.

In one embodiment, the cancer or the disorder is characterized by the presence of a TP53 mutation.

In one embodiment, the cancer or the disorder is characterized by the presence of an IDH2 mutation and a SRSF2 mutation.

In one embodiment, the cancer or the disorder is further characterized by absence of a RAS mutation.

In one embodiment, the human subject has normal karyotype (no abnormalities). In one embodiment, the human subject has abnormal karyotype (1-3 abnormalities). In one embodiment, the human subject has complex karyotype (>4 abnormalities).

In one embodiment, the human subject tests negative for minimal residual disease (MRD). In one embodiment, the human subject tests positive for MRD.

In one embodiment, the cancer or the disorder is characterized by the presence of a NPM1 mutation, and the human subject tests negative for MRD. In one embodiment, the cancer or the disorder is characterized by the presence of a NPM1 mutation, and the human subject tests positive for MRD. In one embodiment, the cancer or the disorder is characterized by the absence of a NPM1 mutation (i.e., characterized as NPM1 wild type), and the human subject tests negative for MRD. In one embodiment, the cancer or the disorder is characterized by the absence of a NPM1 mutation (i.e., characterized as NPM1 wild type), and the human subject tests positive for MRD.

In one embodiment, provided herein is a method of preventing a clonal hematopoiesis of indeterminate potential (CHIP) disease in a human subject from progressing into a cancer or a disorder related to abnormal cell proliferation and/or function, comprising:
(1) identifying the CHIP disease in the human subject to be characterized by:
(i) presence of a DNA (cytosine-5)-methyltransferase 3A (DNMT3A) mutation;
(ii) presence of an additional sex comb-like 1 (ASXL1) mutation; or
(iii) presence of ten-eleven translocation-2 (TET2) mutation; and
(2) administering to the human subject a therapeutically effective amount of a hypomethylating agent.

In one embodiment, the CHIP disease is characterized by the presence of a DNMT3A mutation. In one embodiment, the CHIP disease is characterized by the presence of an ASXL1 mutation. In one embodiment, the CHIP disease is characterized by the presence of a TET2 mutation.

In one embodiment, the cancer or the disorder related to abnormal cell proliferation is a myeloid disease. In one embodiment, the disorder related to abnormal cell proliferation is MDS. In one embodiment, the cancer is AML. In one embodiment, the cancer is diffuse large B-cell lymphoma. In one embodiment, the cancer is primary gastrointestinal diffuse large B-cell lymphoma. In one embodiment, the cancer is T cell lymphoma. In one embodiment, the cancer is multiple myeloma. In one embodiment, the cancer is multiple myeloma undergoing transplant. In one embodiment, the cancer is a solid tumor.

In one embodiment, the hypomethylating agent is administered as the only therapeutic agent or therapy (monotherapy).

In one embodiment, the method further comprises administering at least one additional therapeutic agent or therapy (combination therapy).

In one embodiment, the at least one additional therapeutic agent or therapy is an agent that modulates NPM1 pathway or targets nucleophosmin, a FLT3 inhibitor, an agent or therapy that restore the wild-type DNMT3A function, a PI3K/Akt/mTOR pathway inhibitor, a TP53 inhibitor, an agent that targets spliceosome or its downstream target(s), an IDH2 inhibitor, or a RAS pathway inhibitor.

In one embodiment, the at least one additional therapeutic agent or therapy is an agent that modulates NPM1 pathway or targets nucleophosmin. In one embodiment, the agent that modulates NPM1 pathway or targets nucleophosmin is a DOT1L inhibitor. In one embodiment, the DOT1L inhibitor is SGC0946. In one embodiment, the DOT1L inhibitor is pinometostat. In one embodiment, the agent that modulates NPM1 pathway or target nucleophosmin is a Menin-MLL inhibitor. In one embodiment, the Menin-MLL inhibitor is MI-503.

In one embodiment, the at least one additional therapeutic agent or therapy is a FLT3 inhibitor. Examples of first generation FLT3 inhibitors include but are not limited to midostaurin, lestaurtinib, sunitinib (Sutent®), and sorafenib (Nexavar®). Examples of second generation FLT3 inhibitors include but are not limited to quizartinib, crenolanib, pexidartinib (PLX3397), and gilteritinib (ASP2215), are more potent and selective than the first-generation inhibitors. In one embodiment, the FLT3 inhibitor is midostaurin. In one embodiment, the FLT3 inhibitor is gilteritinib. In one embodiment, the FLT3 inhibitor is quizartinib.

In one embodiment, the at least one additional therapeutic agent or therapy is an agent or therapy that restore the wild-type DNMT3A function. In one embodiment, the agent or therapy that restore the wild-type DNMT3A function is a small molecule compound. In one embodiment, the agent or therapy that restore the wild-type DNMT3A function is a therapy that utilizes genetic method CRISPR.

In one embodiment, the at least one additional therapeutic agent or therapy is a PI3K/Akt/mTOR pathway inhibitor. In one embodiment, the PI3K/Akt/mTOR pathway inhibitor is an mTOR inhibitor. In one embodiment, the mTOR inhibitor is rapamycin or an analog thereof (also termed rapalog). In one embodiment, the mTOR inhibitor is everolimus. In one embodiment, the PI3K/Akt/mTOR pathway inhibitor is a PI3K inhibitor. In one embodiment, the PI3K inhibitor is idelalisib. In one embodiment, the PI3K/Akt/mTOR pathway inhibitor is an Akt inhibitor. In one embodiment, the Akt inhibitor is ipatasertib.

In one embodiment, the at least one additional therapeutic agent or therapy is an agent that targets spliceosome or its downstream target(s). In one embodiment, the agent that targets spliceosome or its downstream target(s) is a SRSF2 inhibitor. In one embodiment, the agent that targets spliceosome or its downstream target(s) is a SF3B1 inhibitor. In one embodiment, the SF3B1 inhibitor is FR901464. In one embodiment, the SF3B1 inhibitor is herboxidiene. In one embodiment, the SF3B1 inhibitor is pladienolide. In one embodiment, the SF3B1 inhibitor is meayamycin. In one embodiment, the SF3B1 inhibitor is E7107. In one embodiment, the SF3B1 inhibitor is spliceostatin A.

In one embodiment, the at least one additional therapeutic agent or therapy is an IDH2 inhibitor. In one embodiment, the IDH2 inhibitor is ivosidenib. In one embodiment, the IDH2 inhibitor is enasidenib. In one embodiment, the IDH2 inhibitor is 2-methyl-1-[(4-[6-(trifluoromethyl)pyridin-2-yl]-6-{[2-(trifluoromethyl)pyridin-4-yl]amino}-1,3,5-triazin-2-yl)amino]propan-2-ol.

In one embodiment, the at least one additional therapeutic agent or therapy is a RAS pathway inhibitor. In one embodiment, the RAS pathway inhibitor is captopril, imidapril, zofenopril, candesartan, delapril, telmisartan, aliskiren, moexipril, enalapril, valsartan, fosinopril, irbesartan, perindopril, quinapril, ramipril, eprosartan, olmesartan, trandolapril, losartan, azilsartan, lisinopril, spirapril, benazepril, or cilazapril. In one embodiment, the RAS pathway inhibitor is a BRAF inhibitor. In one embodiment, the BRAF inhibitor is vemurafenib or dabrafenib. In one embodiment, the RAS pathway inhibitor is a MEK inhibitor. In one embodiment, the MEK inhibitor is trametinib, selumetinib, binimetinib, PD-325901, cobimetinib, CI-1040, or PD035901. In one embodiment, the RAS pathway inhibitor is an ERK inhibitor. In one embodiment, the ERK inhibitor is LY3214996, LTT462, or BVD-523.

In one embodiment, the AML is characterized by the presence of a NPM1 mutation, and the at least one additional therapeutic agent or therapy is an agent that modulates NPM1 pathway or targets nucleophosmin (e.g., a DOT1L inhibitor (e.g., SGC0946 or pinometostat) or a Menin-MLL inhibitor (e.g., MI-503)).

In one embodiment, the AML is characterized by the presence of a NPM1 mutation, and the at least one additional therapeutic agent or therapy is a TP53 inhibitor.

In one embodiment, the AML is characterized by the presence of a NPM1 mutation and a FLT3 mutation, and the at least one additional therapeutic agent or therapy is a FLT3 inhibitor (e.g., midostaurin, gilteritinib, or quizartinib).

In one embodiment, the AML is characterized by the presence of a DNMT3A mutation, and the at least one additional therapeutic agent or therapy is an agent that modulates NPM1 pathway or targets nucleophosmin (e.g., a DOTiL inhibitor (e.g., SGC0946 or pinometostat) or a Menin-MLL inhibitor (e.g., MI-503)).

In one embodiment, the AML is characterized by the presence of a DNMT3A mutation, and the at least one additional therapeutic agent or therapy is an agent or therapy that restore the wild-type DNMT3A function.

In one embodiment, the AML is characterized by the presence of a DNMT3A mutation, and the at least one additional therapeutic agent or therapy is a PI3K/Akt/mTOR pathway inhibitor (e.g., an mTOR inhibitor (e.g., rapamycin or everolimus), a PI3K inhibitor (e.g., idelalisib), or an Akt inhibitor (e.g., ipatasertib)).

In one embodiment, the AML is characterized by the presence of a TP53 mutation, and the at least one additional therapeutic agent or therapy is a TP53 inhibitor.

In one embodiment, the AML is characterized by the presence of an IDH2 mutation and a SRSF2 mutation, and the at least one additional therapeutic agent or therapy is an agent that targets spliceosome or its downstream target(s) (e.g., a SRSF2 inhibitor or a SF3B1 inhibitor (e.g., FR901464, herboxidiene, pladienolide, meayamycin, E7107, or spliceostatin A)).

In one embodiment, the AML is characterized by the presence of an IDH2 mutation and a SRSF2 mutation, and the at least one additional therapeutic agent or therapy is an IDH2 inhibitor (e.g., ivosidenib, enasidenib, or 2-methyl-1-[(4-[6-(trifluoromethyl)pyridin-2-yl]-6-{[2-(trifluoromethyl)pyridin-4-yl]amino}-1,3,5-triazin-2-yl)amino]propan-2-ol).

In one embodiment, the AML is further characterized by the absence of a RAS mutation, and the at least one additional therapeutic agent or therapy is a RAS pathway inhibitor (e.g., a RAS inhibitor (e.g., captopril, imidapril, zofenopril, candesartan, delapril, telmisartan, aliskiren, moexipril, enalapril, valsartan, fosinopril, irbesartan, perindopril, quinapril, ramipril, eprosartan, olmesartan, trandolapril, losartan, azilsartan, lisinopril, spirapril, benazepril, or cilazapril), a BRAF inhibitor (e.g., vemurafenib or dabrafenib), a MEK inhibitor (e.g., trametinib, selumetinib, binimetinib, PD-325901, cobimetinib, CI-1040, or PD035901), or an ERK inhibitor (e.g., LY3214996, LTT462, or BVD-523)).

In one embodiment, the hypomethylating agent (e.g., 5-azacytidine or decitabine) and the at least one additional therapeutic agent or therapy are administered concomitantly. In one embodiment, the hypomethylating agent (e.g., 5-azacytidine or decitabine) and the at least one additional therapeutic agent or therapy are administered sequentially, wherein the hypomethylating agent (e.g., 5-azacytidine or decitabine) is administered first.

In one embodiment, the hypomethylating agent (e.g., 5-azacytidine or decitabine) and the at least one additional therapeutic agent or therapy are co-formulated as a single unit dosage form. In one embodiment, the hypomethylating agent (e.g., 5-azacytidine or decitabine) and the at least one additional therapeutic agent or therapy are formulated as separate dosage forms.

In one embodiment, the at least one additional therapeutic agent or therapy is administered parenterally. In one embodiment, the at least one additional therapeutic agent or therapy is administered orally.

In one embodiment, the hypomethylating agent (e.g., 5-azacytidine or decitabine) and the at least one additional therapeutic agent or therapy provides a synergistic effect to treat the acute myeloid leukemia or myelodysplastic syndrome.

In one embodiment, the hypomethylating agent (e.g., 5-azacytidine or decitabine) is administered before the at least one additional therapeutic agent or therapy.

In one embodiment, the acute myeloid leukemia is resistant to treatment with at least one additional therapeutic agent or therapy. In some embodiments, the acute myeloid leukemia is responsive to treatment with at least one additional therapeutic agent or therapy.

In one embodiment, the human has acute myeloid leukemia. In one embodiment, the human has myelodysplastic syndrome. In one embodiment, the myelodysplastic syndrome is high and very high risk myelodysplastic syndromes as defined by the Revised International Prognostic Scoring System (IPSS-R).

In one embodiment, the cancer is a hematological cancer. In one embodiment, the hematological cancer is acute myeloid leukemia (AML), acute lymphoblastic leukemia (ALL), chronic myeloid leukemia (CML), chronic lymphocytic leukemia (CLL), non-Hodgkin's lymphoma (NHL), Hodgkin's lymphoma, or multiple myeloma (MM).

In one embodiment, the hematological cancer is acute myeloid leukemia (AML). In one embodiment, the AML is de novo AML. In one embodiment, the AML is secondary to prior myelodysplastic disease (MDS) or chronic myelomonocytic leukemia (CMML). In one embodiment, the AML is relapsed or refractory AML. In one embodiment, the AML has intermediate-risk cytogenetic characteristics as defined according to National Comprehensive Cancer Network 2011 guidelines. In one embodiment, the AML has poor-risk cytogenetic characteristics as defined according to National Comprehensive Cancer Network 2011 guidelines.

In one embodiment, the cancer is a solid tumor. In one embodiment, the solid tumor is melanoma, carcinoma, adenocarcinoma, chordoma, breast cancer, colorectal cancer, ovarian cancer, lung cancer, testicular cancer, renal cancer, pancreatic cancer, bone cancer, gastric cancer, head and neck cancer, or prostate cancer.

In one embodiment, the disorder related to abnormal cell proliferation is myelodysplastic syndromes (MDS).

In one embodiment, the subject is a human.

### IV. Method of Identifying Subjects Responsive to Treatment

Provided herein are methods for identifying subjects with cancer or disorders related to abnormal cell proliferation who are likely to be responsive to treatment with a hypomethylating agent.

In one embodiment, provided herein is a method of treating a cancer or a disorder related to abnormal cell proliferation in a human subject with a hypomethylating agent, comprising:
(a) identifying the human subject having the cancer or the disorder that may be responsive to the treatment comprising the hypomethylating agent, comprising:
i. providing a sample from the human subject;
ii. detecting the presence of one or more gene mutations in the sample; and
iii. identifying the human subject as being likely to be responsive to the treatment comprising the hypomethylating agent if one or more gene mutations are detected; and
(b) administering to the human subject a therapeutically effective amount of the hypomethylating agent if the human subject is identified as being likely to be responsive to the treatment.

In one embodiment, provided herein is a method of treating a cancer or a disorder related to abnormal cell proliferation in a human subject with a hypomethylating agent, comprising:
(a) identifying the human subject having the cancer or the disorder that may be responsive to the treatment comprising the hypomethylating agent, comprising:
i. detecting the presence of one or more gene mutations in a sample obtained from the patient; and
ii. identifying the human subject as being likely to be responsive to the treatment comprising the hypomethylating agent if one or more gene mutations are detected; and
(b) administering to the human subject a therapeutically effective amount of the hypomethylating agent if the human subject is identified as being likely to be responsive to the treatment.

In one embodiment, provided herein is a method of identifying a human subject having a cancer or a disorder related to abnormal cell proliferation that may be responsive to the treatment comprising a hypomethylating agent, comprising:
detecting the presence of one or more gene mutations in a sample obtained from the patient;
wherein the patient is likely to be responsive to the treatment with the hypomethylating agent if the one or more gene mutations are detected.

In one embodiment, provided herein is a method of identifying a human subject having a cancer or a disorder related to abnormal cell proliferation that may be responsive to the treatment comprising a hypomethylating agent, comprising:
detecting the presence of one or more gene mutations in a sample obtained from the patient; and
identifying the subject as likely to be responsive to the hypomethylating agent based on the presence or the one or more gene mutations in the sample.

In one embodiment, provided herein is a method of diagnosing the responsiveness of a a human subject having a cancer or a disorder related to abnormal cell proliferation to treatment with a hypomethylating agent, comprising:
detecting the presence of one or more gene mutations in a sample obtained from the patient; and
diagnosing the subject a likely to be responsive to the hypomethylating agent based on the presence or the one or more gene mutations in the sample.

In one embodiment, the one or more gene mutations are detected in NPM1, FLT3, DNMT3A, TP53, IDH2 and/or SRSF.

In one embodiment, provided herein is a method of treating a cancer or a disorder related to abnormal cell proliferation in a human subject with a hypomethylating agent, comprising:
(a) identifying the human subject having the cancer or the disorder that may be responsive to the treatment comprising the hypomethylating agent, comprising:
i. providing a sample from the human subject;
ii. detecting the presence of one or more gene mutations in the sample; and
iii. identifying the human subject as being likely to be responsive to the treatment comprising the hypomethylating agent if one or more gene mutations are detected; and
(b) administering to the human subject a therapeutically effective amount of the hypomethylating agent if the human subject is identified as being likely to be responsive to the treatment;
wherein the one or more gene mutations are detected in NPM1, FLT3, DNMT3A, TP53, IDH2 and/or SRSF2.

In one embodiment, provided herein is a method of treating a cancer or a disorder related to abnormal cell proliferation in a human subject with a hypomethylating agent, comprising:
(a) identifying the human subject having the cancer or the disorder that may be responsive to the treatment comprising the hypomethylating agent, comprising:
i. detecting the presence of one or more gene mutations in a sample obtained from the patient; and
ii. identifying the human subject as being likely to be responsive to the treatment comprising the hypomethylating agent if one or more gene mutations are detected;
(b) administering to the human subject a therapeutically effective amount of the hypomethylating agent if the human subject is identified as being likely to be responsive to the treatment;
wherein the one or more gene mutations are detected in NPM1, FLT3, DNMT3A, TP53, IDH2 and/or SRSF2.

In one embodiment, provided herein is a method of identifying a human subject having a cancer or a disorder related to abnormal cell proliferation that may be responsive to the treatment comprising a hypomethylating agent, comprising:
detecting the presence of one or more gene mutations in a sample obtained from the patient;
wherein the patient is likely to be responsive to the treatment with the hypomethylating agent if the one or more gene mutations are detected; and
wherein the one or more gene mutations are detected in NPM1, FLT3, DNMT3A, TP53, IDH2 and/or SRSF2.

In one embodiment, provided herein is a method of identifying a human subject having a cancer or a disorder related to abnormal cell proliferation that may be responsive to the treatment comprising a hypomethylating agent, comprising:
detecting the presence of one or more gene mutations in a sample obtained from the patient;
identifying the subject as likely to be responsive to the hypomethylating agent based on the presence or the one or more gene mutations in the sample; and
wherein the one or more gene mutations are detected in NPM1, FLT3, DNMT3A, TP53, IDH2 and/or SRSF2.

In one embodiment, provided herein is a method of diagnosing the responsiveness of a a human subject having a cancer or a disorder related to abnormal cell proliferation to treatment with a hypomethylating agent, comprising:
detecting the presence of one or more gene mutations in a sample obtained from the patient;
diagnosing the subject a likely to be responsive to the hypomethylating agent based on the presence or the one or more gene mutations in the sample; and
wherein the one or more gene mutations are detected in NPM1, FLT3, DNMT3A, TP53, IDH2 and/or SRSF2.

In one embodiment, the presence of the one or more gene mutations is detected by sequencing, for example, targeted next-generation sequencing. In one embodiment, the presence of the one or more gene mutations is detected in DNA isolated from the patient's sample. In one embodiment, the patient's sample comprises tumor cells.

In one embodiment, the presence of a NPM1 mutation is detected. In one embodiment, the presence of a FLT3 mutation is detected. In one embodiment, the presence of a NPM1 mutation and a FLT3 mutation is detected.

In one embodiment, the FLT3 mutation is a FMS-like tyrosine kinase-3 internal tandem duplication (FLT3-ITD) or FMS-like tyrosine kinase-3 tyrosine kinase domain (FLT3-TKD) mutation. In one embodiment, the FLT3 mutation is a FLT3-ITD mutation. In one embodiment, the FLT3 mutation is a FLT3-TKD mutation. In one embodiment, the FLT3 mutation is a FLT3-ITD and FLT3-TKD co-mutation.

In one embodiment, the presence of a DNMT3A mutation is detected.

In one embodiment, the presence of a TP53 mutation is detected.

In one embodiment, the presence of an IDH2 mutation and a SRSF2 mutation is detected.

In one embodiment, the presence of a RAS mutation is not detected.

In one embodiment, the human subject has normal karyotype (no abnormalities). In one embodiment, the human subject has abnormal karyotype (1-3 abnormalities). In one embodiment, the human subject has complex karyotype (>4 abnormalities).

In one embodiment, the human subject tests negative for minimal residual disease (MRD). In one embodiment, the human subject tests positive for MRD.

In one embodiment, the presence of a NPM1 mutation is detected, and the human subject tests negative for MRD. In one embodiment, the presence of a NPM1 mutation is detected, and the human subject tests positive for MRD. In one embodiment, the presence of a NPM1 mutation is not detected (i.e., only NPM1 wild type is detected), and the human subject tests negative for MRD. In one embodiment, the presence of a NPM1 mutation is not detected (i.e., only NPM1 wild type is detected), and the human subject tests positive for MRD.

In one embodiment, the hypomethylating agent is 5-azacytidine or decitabine.

In one embodiment, the hypomethylating agent is administered as the only therapeutic agent or therapy (monotherapy).

In one embodiment, the method further comprises administering at least one additional therapeutic agent or therapy (combination therapy).

In one embodiment, the at least one additional therapeutic agent or therapy is an agent that modulates NPM1 pathway or targets nucleophosmin, a FLT3 inhibitor, an agent or therapy that restore the wild-type DNMT3A function, a PI3K/Akt/mTOR pathway inhibitor, a TP53 inhibitor, an agent that targets spliceosome or its downstream target(s), an IDH2 inhibitor, or a RAS pathway inhibitor.

In one embodiment, the at least one additional therapeutic agent or therapy is an agent that modulates NPM1 pathway or targets nucleophosmin. In one embodiment, the agent that modulates NPM1 pathway or targets nucleophosmin is a DOT1L inhibitor. In one embodiment, the DOT1L inhibitor is SGC0946. In one embodiment, the DOT1L inhibitor is pinometostat. In one embodiment, the agent that modulates NPM1 pathway or target nucleophosmin is a Menin-MLL inhibitor. In one embodiment, the Menin-MLL inhibitor is MI-503.

In one embodiment, the at least one additional therapeutic agent or therapy is a FLT3 inhibitor. Examples of first generation FLT3 inhibitors include but are not limited to midostaurin, lestaurtinib, sunitinib (Sutent®), and sorafenib (Nexavar®). Examples of second generation FLT3 inhibitors include but are not limited to quizartinib, crenolanib, pexidartinib (PLX3397), and gilteritinib (ASP2215), are more potent and selective than the first-generation inhibitors. In one embodiment, the FLT3 inhibitor is midostaurin. In one embodiment, the FLT3 inhibitor is gilteritinib. In one embodiment, the FLT3 inhibitor is quizartinib.

In one embodiment, the at least one additional therapeutic agent or therapy is an agent or therapy that restore the wild-type DNMT3A function. In one embodiment, the agent or therapy that restore the wild-type DNMT3A function is a small molecule compound. In one embodiment, the agent or therapy that restore the wild-type DNMT3A function is a therapy that utilizes genetic method CRISPR.

In one embodiment, the at least one additional therapeutic agent or therapy is a PI3K/Akt/mTOR pathway inhibitor. In one embodiment, the PI3K/Akt/mTOR pathway inhibitor is an mTOR inhibitor. In one embodiment, the mTOR inhibitor is rapamycin or an analog thereof (also termed rapalog). In one embodiment, the mTOR inhibitor is everolimus. In one embodiment, the PI3K/Akt/mTOR pathway inhibitor is a PI3K inhibitor. In one embodiment, the PI3K inhibitor is idelalisib. In one embodiment, the PI3K/Akt/mTOR pathway inhibitor is an Akt inhibitor. In one embodiment, the Akt inhibitor is ipatasertib.

In one embodiment, the at least one additional therapeutic agent or therapy is an agent that targets spliceosome or its downstream target(s). In one embodiment, the agent that targets spliceosome or its downstream target(s) is a SRSF2 inhibitor. In one embodiment, the agent that targets spliceosome or its downstream target(s) is a SF3B1 inhibitor. In one embodiment, the SF3B1 inhibitor is FR901464. In one embodiment, the SF3B1 inhibitor is herboxidiene. In one embodiment, the SF3B1 inhibitor is pladienolide. In one embodiment, the SF3B1 inhibitor is meayamycin. In one embodiment, the SF3B1 inhibitor is E7107. In one embodiment, the SF3B1 inhibitor is spliceostatin A.

In one embodiment, the at least one additional therapeutic agent or therapy is an IDH2 inhibitor. In one embodiment, the IDH2 inhibitor is ivosidenib. In one embodiment, the IDH2 inhibitor is enasidenib. In one embodiment, the IDH2 inhibitor is 2-methyl-1-[(4-[6-(trifluoromethyl)pyridin-2-yl]-6-{[2-(trifluoromethyl)pyridin-4-yl]amino}-1,3,5-triazin-2-yl)amino]propan-2-ol.

In one embodiment, the at least one additional therapeutic agent or therapy is a RAS pathway inhibitor. In one embodiment, the RAS pathway inhibitor is captopril, imidapril, zofenopril, candesartan, delapril, telmisartan, aliskiren, moexipril, enalapril, valsartan, fosinopril, irbesartan, perindopril, quinapril, ramipril, eprosartan, olmesartan, trandolapril, losartan, azilsartan, lisinopril, spirapril, benazepril, or cilazapril. In one embodiment, the RAS pathway inhibitor is a BRAF inhibitor. In one embodiment, the BRAF inhibitor is vemurafenib or dabrafenib. In one embodiment, the RAS pathway inhibitor is a MEK inhibitor. In one embodiment, the MEK inhibitor is trametinib, selumetinib, binimetinib, PD-325901, cobimetinib, CI-1040, or PD035901. In one embodiment, the RAS pathway inhibitor is an ERK inhibitor. In one embodiment, the ERK inhibitor is LY3214996, LTT462, or BVD-523.

In one embodiment, the cancer or the disorder is characterized by the presence of a NPM1 mutation, and the at least one additional therapeutic agent or therapy is an agent that modulates NPM1 pathway or targets nucleophosmin (e.g., a DOTiL inhibitor (e.g., SGC0946 or pinometostat) or a Menin-MLL inhibitor (e.g., MI-503)).

In one embodiment, the cancer or the disorder is characterized by the presence of a NPM1 mutation, and the at least one additional therapeutic agent or therapy is a TP53 inhibitor.

In one embodiment, the cancer or the disorder is characterized by the presence of a NPM1 mutation and a FLT3 mutation, and the at least one additional therapeutic agent or therapy is a FLT3 inhibitor (e.g., midostaurin, gilteritinib, or quizartinib).

In one embodiment, the cancer or the disorder is characterized by the presence of a DNMT3A mutation, and the at least one additional therapeutic agent or therapy is an agent that modulates NPM1 pathway or targets nucleophosmin (e.g., a DOTiL inhibitor (e.g., SGC0946 or pinometostat) or a Menin-MLL inhibitor (e.g., MI-503)).

In one embodiment, the cancer or the disorder is characterized by the presence of a DNMT3A mutation, and the at least one additional therapeutic agent or therapy is an agent or therapy that restore the wild-type DNMT3A function.

In one embodiment, the cancer or the disorder is characterized by the presence of a DNMT3A mutation, and the at least one additional therapeutic agent or therapy is a PI3K/Akt/mTOR pathway inhibitor (e.g., an mTOR inhibitor (e.g., rapamycin or everolimus), a PI3K inhibitor (e.g., idelalisib), or an Akt inhibitor (e.g., ipatasertib)).

In one embodiment, the cancer or the disorder is characterized by the presence of a TP53 mutation, and the at least one additional therapeutic agent or therapy is a TP53 inhibitor.

In one embodiment, the cancer or the disorder is characterized by the presence of an IDH2 mutation and a SRSF2 mutation, and the at least one additional therapeutic agent or therapy is an agent that targets spliceosome or its downstream target(s) (e.g., a SRSF2 inhibitor or a SF3B1 inhibitor (e.g., FR901464, herboxidiene, pladienolide, meayamycin, E7107, or spliceostatin A)).

In one embodiment, the cancer or the disorder is characterized by the presence of an IDH2 mutation and a SRSF2 mutation, and the at least one additional therapeutic agent or therapy is an IDH2 inhibitor (e.g., ivosidenib, enasidenib, or 2-methyl-1-[(4-[6-(trifluoromethyl)pyridin-2-yl]-6-{[2-(trifluoromethyl)pyridin-4-yl]amino}-1,3,5-triazin-2-yl)amino]propan-2-ol).

In one embodiment, the cancer or the disorder is further characterized by the absence of a RAS mutation, and the at least one additional therapeutic agent or therapy is a RAS pathway inhibitor (e.g., a RAS inhibitor (e.g., captopril, imidapril, zofenopril, candesartan, delapril, telmisartan, aliskiren, moexipril, enalapril, valsartan, fosinopril, irbesartan, perindopril, quinapril, ramipril, eprosartan, olmesartan, trandolapril, losartan, azilsartan, lisinopril, spirapril, benazepril, or cilazapril), a BRAF inhibitor (e.g., vemurafenib or dabrafenib), a MEK inhibitor (e.g., trametinib, selumetinib, binimetinib, PD-325901, cobimetinib, CI-1040, or PD035901), or an ERK inhibitor (e.g., LY3214996, LTT462, or BVD-523)).

In one embodiment, the hypomethylating agent (e.g., 5-azacytidine or decitabine) and the at least one additional therapeutic agent or therapy are administered concomitantly. In one embodiment, the hypomethylating agent (e.g., 5-azacytidine or decitabine) and the at least one additional therapeutic agent or therapy are administered sequentially, wherein the hypomethylating agent (e.g., 5-azacytidine or decitabine) is administered first.

In one embodiment, the hypomethylating agent (e.g., 5-azacytidine or decitabine) and the at least one additional therapeutic agent or therapy are co-formulated as a single unit dosage form. In one embodiment, the hypomethylating agent (e.g., 5-azacytidine or decitabine) and the at least one additional therapeutic agent or therapy are formulated as separate dosage forms.

In one embodiment, the at least one additional therapeutic agent or therapy is administered parenterally. In one embodiment, the at least one additional therapeutic agent or therapy is administered orally.

In one embodiment, the hypomethylating agent (e.g., 5-azacytidine or decitabine) and the at least one additional therapeutic agent or therapy provides a synergistic effect to treat the acute myeloid leukemia or myelodysplastic syndrome.

In one embodiment, the hypomethylating agent (e.g., 5-azacytidine or decitabine) is administered before the at least one additional therapeutic agent or therapy.

In one embodiment, the acute myeloid leukemia is resistant to treatment with at least one additional therapeutic agent or therapy. In some embodiments, the acute myeloid leukemia is responsive to treatment with at least one additional therapeutic agent or therapy.

In one embodiment, the human has acute myeloid leukemia. In one embodiment, the human has myelodysplastic syndrome. In one embodiment, the myelodysplastic syndrome is high and very high risk myelodysplastic syndromes as defined by the Revised International Prognostic Scoring System (IPSS-R).

In one embodiment, the cancer is a hematological cancer. In one embodiment, the hematological cancer is acute myeloid leukemia (AML), acute lymphoblastic leukemia (ALL), chronic myeloid leukemia (CML), chronic lymphocytic leukemia (CLL), non-Hodgkin's lymphoma (NHL), Hodgkin's lymphoma, or multiple myeloma (MM).

In one embodiment, the hematological cancer is acute myeloid leukemia (AML). In one embodiment, the AML is de novo AML. In one embodiment, the AML is secondary to prior myelodysplastic disease (MDS) or chronic myelomonocytic leukemia (CMML). In one embodiment, the AML is relapsed or refractory AML. In one embodiment, the AML has intermediate-risk cytogenetic characteristics as defined according to National Comprehensive Cancer Network 2011 guidelines. In one embodiment, the AML has poor-risk cytogenetic characteristics as defined according to National Comprehensive Cancer Network 2011 guidelines.

In one embodiment, the cancer is a solid tumor. In one embodiment, the solid tumor is melanoma, carcinoma, adenocarcinoma, chordoma, breast cancer, colorectal cancer, ovarian cancer, lung cancer, testicular cancer, renal cancer, pancreatic cancer, bone cancer, gastric cancer, head and neck cancer, or prostate cancer.

In one embodiment, the disorder related to abnormal cell proliferation is myelodysplastic syndromes (MDS).

In one embodiment, the subject is a human.

### V. Hypomethylating Agent and Additional Therapeutic Agents

In one embodiment, the DNMT inhibitor/hypomethylating agent used in the methods provided herein is a nucleoside inhibitor. In one embodiment, the nucleoside inhibitor is 5-azacytidine, decitabine, guadecitabine, or zebularine.

In one embodiment, the DNMT inhibitor/hypomethylating agent used in the methods provided herein is a non-nucleoside inhibitor. In one embodiment, the non-nucleoside inhibitor is SGI-1027, RG108, DC_05, or GSK3482364.

In one embodiment, the hypomethylating agent is 5-azacytidine. In one embodiment, the 5-azacytidine is in orally administered as a composition that is non-enteric-coated.

In one embodiment, the hypomethylating agent is decitabine.

In one embodiment, the hypomethylating agent (e.g., 5-azacytidine or decitabine) is administered at a dose of about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, or about 600 mg. In one embodiment, the hypomethylating agent (e.g., 5-azacytidine or decitabine) is administered at a dose of about 200 mg. In one embodiment, the hypomethylating agent (e.g., 5-azacytidine or decitabine) is administered at a dose of about 300 mg. In one embodiment, the hypomethylating agent (e.g., 5-azacytidine or decitabine) is administered at a dose of 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg, 400 mg, 410 mg, 420 mg, 430 mg, 440 mg, 450 mg, 460 mg, 470 mg, 480 mg, 490 mg, 500 mg, 510 mg, 520 mg, 530 mg, 540 mg, 550 mg, 560 mg, 570 mg, 580 mg or 600 mg orally. In one embodiment, the hypomethylating agent (e.g., 5-azacytidine or decitabine) is administered at a dose of 200 to 300 mg orally. In one embodiment, the hypomethylating agent (e.g., 5-azacytidine or decitabine) is administered at a dose of 200 mg. In one embodiment, the hypomethylating agent (e.g., 5-azacytidine or decitabine) is administered at a dose of 300 mg. In one embodiment, the hypomethylating agent (e.g., 5-azacytidine or decitabine) is administered for the first seven, fourteen, or twenty-one days of a 28-day cycle. In one embodiment, the hypomethylating agent (e.g., 5-azacytidine or decitabine) is administered to the human subject one or two times per day. In one embodiment, the hypomethylating agent (e.g., 5-azacytidine or decitabine) is administered to the human subject once per day. In one embodiment, the hypomethylating agent (e.g., 5-azacytidine or decitabine) is administered in the form of a capsule or a tablet. In one embodiment, the hypomethylating agent (e.g., 5-azacytidine or decitabine) is administered in the form of a non-enteric-coated tablet. In one embodiment, the hypomethylating agent (e.g., 5-azacytidine or decitabine) is administered in the form of an immediate release oral composition.

In one embodiment, the hypomethylating agent (e.g., 5-azacytidine or decitabine) is administered at a dose of about 200 mg per day for 14 days in a 28-day cycle. In one embodiment, the hypomethylating agent (e.g., 5-azacytidine or decitabine) is administered at a dose of about 300 mg per day for 14 days in a 28-day cycle. In one embodiment, the hypomethylating agent (e.g., 5-azacytidine or decitabine) is administered at a dose of about 200 mg per day for 21 days in a 28-day cycle. In one embodiment, the hypomethylating agent (e.g., 5-azacytidine or decitabine) is administered at a dose of about 300 mg per day for 21 days in a 28-day cycle. In one embodiment, the hypomethylating agent (e.g., 5-azacytidine or decitabine) is administered at a dose of about 200 mg per day for 7 days in a 28-day cycle. In one embodiment, the hypomethylating agent (e.g., 5-azacytidine or decitabine) is administered at a dose of about 300 mg per day for 7 days in a 28-day cycle. In one embodiment, the hypomethylating agent (e.g., 5-azacytidine or decitabine) is administered at a dose of 200 mg per day for 14 days in a 28-day cycle. In one embodiment, the hypomethylating agent (e.g., 5-azacytidine or decitabine) is administered at a dose of 300 mg per day for 14 days in a 28-day cycle. In one embodiment, the hypomethylating agent (e.g., 5-azacytidine or decitabine) is administered at a dose of 200 mg per day for 21 days in a 28-day cycle. In one embodiment, the hypomethylating agent (e.g., 5-azacytidine or decitabine) is administered at a dose of 300 mg per day for 21 days in a 28-day cycle. In one embodiment, the hypomethylating agent (e.g., 5-azacytidine or decitabine) is administered at a dose of 200 mg per day for 7 days in a 28-day cycle. In one embodiment, the hypomethylating agent (e.g., 5-azacytidine or decitabine) is administered at a dose of 300 mg per day for 7 days in a 28-day cycle.

In one embodiment, the hypomethylating agent (e.g., 5-azacytidine or decitabine) is administered: (a) daily for 1, 2, 3, 4, 5, 6, 7, 8, 9, 10, 11, 12, 13, 14, or greater than 14 days, optionally followed by a treatment dosing holiday of 1, 2, 3, 4, 5, 6, 7, 8, 9, 10, 11, 12, 13, 14, or greater than 14 days; (b) daily for 14 or more days, optionally followed by a treatment dosing holiday of 7 or more days; (c) for 21 or more days, optionally followed by a treatment dosing holiday of 7 or more days; (d) for 14 days, optionally followed by a treatment dosing holiday of 14 days; (e) for 21 or more days, followed by a treatment dosing holiday of 7 or more days; or (f) for 14 days, followed by a treatment dosing holiday of 14 days. In one embodiment, at least one of steps (a), (b), (c), (d), (e), or (f) are repeated.

In one embodiment, the hypomethylating agent (e.g., 5-azacytidine or decitabine) is administered: (a) at a dose of about 300 mg daily for 14 days, followed by a treatment dosing holiday of 14 days; (b) at a dose of about 200 mg daily for 14 days, followed by a treatment dosing holiday of 14 days; (c) at a dose of about 300 mg daily for 21 days, followed by a treatment dosing holiday of 7 days; (d) at a dose of about 200 mg daily for 21 days, followed by a treatment dosing holiday of 7 days; (e) at a dose of 300 mg daily for 14 days, followed by a treatment dosing holiday of 14 days; (f) at a dose of 200 mg daily for 14 days, followed by a treatment dosing holiday of 14 days; (g) at a dose of 300 mg daily for 21 days, followed by a treatment dosing holiday of 7 days; or (h) at a dose of 200 mg daily for 21 days, followed by a treatment dosing holiday of 7 days. In one embodiment, at least one of steps (a), (b), (c), (d), (e), (f), (g), or (h) are repeated.

In one embodiment, the hypomethylating agent (e.g., 5-azacytidine or decitabine) is administered using a treatment cycle comprising administration of the hypomethylating agent (e.g., 5-azacytidine or decitabine) per day for 7 days in a 28-day cycle. In one embodiment, the hypomethylating agent (e.g., 5-azacytidine or decitabine) is administered using a treatment cycle comprising administration of the hypomethylating agent (e.g., 5-azacytidine or decitabine) per day for 14 days in a 28-day cycle. In one embodiment, the hypomethylating agent (e.g., 5-azacytidine or decitabine) is administered using a treatment cycle comprising administration of the hypomethylating agent (e.g., 5-azacytidine or decitabine) per day for 21 days in a 28-day cycle.

In one embodiment, the hypomethylating agent (e.g., 5-azacytidine or decitabine) is administered using a treatment cycle comprising administration of the hypomethylating agent (e.g., 5-azacytidine or decitabine) per day for 7 days followed by 21 days of rest in a 28-day cycle. In one embodiment, the hypomethylating agent (e.g., 5-azacytidine or decitabine) is administered using a treatment cycle comprising administration of the hypomethylating agent (e.g., 5-azacytidine or decitabine) per day for 14 days followed by 14 days of rest in a 28-day cycle. In one embodiment, the hypomethylating agent (e.g., 5-azacytidine or decitabine) is administered using a treatment cycle comprising administration of the hypomethylating agent (e.g., 5-azacytidine or decitabine) per day for 21 days followed by 7 days of rest in a 28-day cycle.

In one embodiment, the hypomethylating agent (e.g., 5-azacytidine or decitabine) is administered parenterally. In one embodiment, the hypomethylating agent (e.g., 5-azacytidine or decitabine) is administered intravenously. In one embodiment, the hypomethylating agent (e.g., 5-azacytidine or decitabine) is administered orally.

Hypomethylating Agent with at Least One Additional Therapeutic Agent

In particular embodiments, the hypomethylating agent (e.g., 5-azacytidine or decitabine) compositions provided herein further comprise one, two, three, or more other pharmacologically active substances (also termed herein “additional therapeutic agents,” “second active agents,” or the like). In some embodiments, the hypomethylating agent (e.g., 5-azacytidine or decitabine) compositions are oral formulations. In some embodiments, the hypomethylating agent (e.g., 5-azacytidine or decitabine) oral compositions with at least one additional therapeutic agent is used for treating any of the diseases or disorders provided herein. In particular embodiments, the oral formulations provided herein comprise the additional therapeutic agent(s) in a therapeutically effective amount. In particular embodiments, the hypomethylating agent (e.g., 5-azacytidine or decitabine) and the additional therapeutic agent(s) are co-formulated together in the same dosage form using methods of co-formulating active pharmaceutical ingredients, including methods disclosed herein and methods known in the art. In other embodiments, the hypomethylating agent (e.g., 5-azacytidine or decitabine) and the additional therapeutic agent(s) are co-administered in separate dosage forms. In some embodiments, certain combinations work synergistically in the treatment of particular diseases or disorders, including, e.g., types of cancer and certain diseases and conditions associated with, or characterized by, undesired angiogenesis or abnormal cell proliferation.

Examples of additional therapeutic agents include but are not limited to an agent that modulates NPM1 pathway or targets nucleophosmin, a FLT3 inhibitor, an agent or therapy that restore the wild-type DNMT3A function, a PI3K/Akt/mTOR pathway inhibitor, a TP53 inhibitor, an agent that targets spliceosome or its downstream target(s), an IDH2 inhibitor, or a RAS pathway inhibitor, as provided herein.

Gilteritinib is a tyrosine kinase inhibitor and is marketed as XOSPATA®, which is in the form of a tablet. Gilteritinib is indicated in the US for the treatment of adult patients who have relapsed or refractory acute myeloid leukemia (AML) with a FLT3 mutation as detected by an FDA-approved test. The recommended starting dose for gilteritinib is 120 mg orally once daily with or without food.

In some embodiments, the gilteritinib is administered orally. In some embodiments, the gilteritinib is administered in a form of a tablet. In some embodiments, the gilteritinib is administered daily. In some embodiments, the gilteritinib is administered at a dose of from about 20 mg to about 400 mg, from about 40 mg to about 400 mg, from about 40 mg to about 200 mg, such as about 20 mg, about 40 mg, about 50 mg, about 80 mg, about 100 mg, about 120 mg, about 160 mg, about 200 mg, or about 400 mg. In some embodiments, the gilteritinib is administered at a dose of about 120 mg.

In some embodiments, the hypomethylating agent (e.g., 5-azacytidine or decitabine) and gilteritinib are administered concomitantly. In some embodiments, the hypomethylating agent (e.g., 5-azacytidine or decitabine) and gilteritinib are administered sequentially. In some embodiments, where the hypomethylating agent (e.g., 5-azacytidine or decitabine) and gilteritinib are administered sequentially, the hypomethylating agent (e.g., 5-azacytidine or decitabine) is administered first. In some embodiments, the hypomethylating agent (e.g., 5-azacytidine or decitabine) and gilteritinib are administered as separate dosage forms, such as injections suitable for intravenous or subcutaneous use and/or tablets or capsules for oral use. In some embodiments, the hypomethylating agent (e.g., 5-azacytidine or decitabine) and gilteritinib are co-formulated as a single unit dosage form, such as an injection suitable for intravenous or subcutaneous use or a tablet or capsule for oral use.

As described herein, certain embodiments herein provide methods of treating a human subject having acute myeloid leukemia (AML), wherein the method includes administering to the human subject (i) a pharmaceutical composition comprising a hypomethylating agent (e.g., 5-azacytidine or decitabine); and (ii) at least one additional therapeutic agent. In some embodiments, the pharmaceutical composition comprising the hypomethylating agent (e.g., 5-azacytidine or decitabine) is administered orally. Also in some embodiments, the pharmaceutical composition comprising a hypomethylating agent (e.g., 5-azacytidine or decitabine) as described herein is used with at least one additional therapeutic agent are used for treating AML in a subject, including a human patient.

In some embodiments, the hypomethylating agent (e.g., 5-azacytidine or decitabine) and one or more therapeutic agents are co-administered to human subjects to yield a synergistic therapeutic effect. The co-administered agent may be a cancer therapeutic agent dosed orally or by injection.

In certain embodiments, methods provided herein for treating disorders related to abnormal cell proliferation comprise orally administering a formulation comprising a therapeutically effective amount of hypomethylating agent (e.g., 5-azacytidine or decitabine). Particular therapeutic indications relating to the methods provided herein are disclosed herein. In certain embodiments, the therapeutically effective amount of the hypomethylating agent (e.g., 5-azacytidine or decitabine) in the pharmaceutical formulation is an amount as provided herein. In certain embodiments, the precise therapeutically effective amount of the hypomethylating agent (e.g., 5-azacytidine or decitabine) in the pharmaceutical formulation will vary depending on, e.g., the age, weight, disease and/or condition of the human subject.

In particular embodiments, the disorders related to abnormal cell proliferation include, but are not limited to, myelodysplastic syndrome (MDS), acute myeloid leukemia (AML), acute lymphoblastic leukemia (ALL), chronic myeloid leukemia (CMIL), leukemia, chronic lymphocytic leukemia (CLL), lymphoma (including non-Hodgkin's lymphoma (NHL) and Hodgkin's lymphoma), multiple myeloma (MM), sarcoma, melanoma, carcinoma, adenocarcinoma, chordoma, breast cancer, colorectal cancer, ovarian cancer, lung cancer (e.g., non-small-cell lung cancer and small-cell lung cancer), testicular cancer, renal cancer, pancreatic cancer, bone cancer, gastric cancer, head and neck cancer, and prostate cancer. In particular embodiments, the disorder related to abnormal cell proliferation is lymphoma. In particular embodiments, the lymphoma is angioimmunoblastic T-cell lymphoma. In particular embodiments, the disorder related to abnormal cell proliferation is MDS. In particular embodiments, the disorder related to abnormal cell proliferation is AML.

Particular embodiments herein provide methods for treating a human subject having a disease or disorder provided herein by orally administering a pharmaceutical composition provided herein, wherein the treatment results in improved survival of the patient. In certain embodiments, the improved survival is measured as compared to one or more standard care regimens. Particular embodiments herein provide methods for treating a human subject having a disease or disorder provided herein by orally administering a pharmaceutical composition provided herein, wherein the treatment provides improved effectiveness for treating the disease or disorder. In particular embodiments, the improved effectiveness is measured using one or more endpoints for cancer clinical trials, as recommended by the U.S. Food and Drug Administration (FDA). For example, FDA provides Guidance for Industry on Clinical Trial Endpoints for the Approval of Cancer Drugs and Biologics (http://www.fda.gov/CbER/gdlns/clintrialend.htm). The FDA endpoints include, but are not limited to, Overall Survival, Endpoints Based on Tumor Assessments such as (i) Disease-Free Survival (ii) Objective Response Rate, (iii) Time to Progression and Progression-Free Survival and (iv) Time-to-Treatment Failure. Endpoints Involving Symptom Endpoints may include Specific Symptom Endpoints such as (i) Time to progression of cancer symptoms and (ii) A composite symptom endpoint. Biomarkers assayed from blood or body fluids may also be useful to determine the management of the disease. In some embodiments, the improvement can be about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

Subjects in need of treatment can be members of a patient population with an increased risk of AML. For example, several inherited genetic disorders and immunodeficiency states are associated with an increased risk of AML. These include disorders with defects in DNA stability, leading to random chromosomal breakage, such as Bloom's syndrome, Fanconi's anemia, Li-Fraumeni kindreds, ataxia-telangiectasia, and X-linked agammaglobulinemia.

In certain embodiments, methods provided herein comprise treating acute promyelocytic leukaemia (APML) by administering a pharmaceutical composition comprising a hypomethylating agent (e.g., 5-azacytidine or decitabine) in combination with one or more additional agents to a human subject in need thereof. APML is a rare sub-type of AML and is sometimes referred to as AML M31. This subtype is characterized by promyelocytic blasts containing the 15;17 chromosomal translocation. This translocation leads to the generation of the fusion transcript comprised of the retinoic acid receptor and a sequence PML.

In some embodiments, methods described herein are used to treat specific types of acute myeloid leukemia. Illustrative types of acute myeloid leukemia include but are not limited to, acute myeloid leukemia with recurrent genetic abnormalities, acute myeloid leukemia with myelodysplasia-related changes, therapy-related myeloid neoplasms, myeloid sarcoma, myeloid proliferations related to Down syndrome, blastic plasmacytoid dendritic cell neoplasm, and/or acute promyelocytic leukaemia.

In some embodiments, the AML is characterized as caused by any one of the following mutations: Fms-related tyrosine kinase 3 (FLT3), Kirsten rat sarcoma viral oncogene homolog (KRAS), neuroblastoma RAS viral (V-Ras) oncogene homolog (NRAS), proto-oncogene c-Kit (KIT), protein tyrosine phosphatase non-receptor type 11 (PTPN11), neurofibromin 1 (NF1), DNA methyltransferase 3A (DNMT3A), isocitrate dehydrogenase 1 (IDH1), isocitrate dehydrogenase 2 (IDH2), ten-eleven translocation-2 (TET2), additional sex comb-like 1 (ASXL1), enhancer of zeste homolog 2 (EZH2), mixed-lineage leukemia 1/histone-lysine N-methyltransferase 2A (MLL/KMT2A), nucleophosmin (NPM1), CCAAT enhancer binding protein alpha (CEBPA), runt-related transcription factor 1 (RUNX1), GATA-binding factor 2 (GATA2), tumor protein p53 (TP53), serine-and-arginine-rich-splicing-factor 2 (SRSF2), U2 small nuclear RNA auxiliary factor 1 (U2AF1), splicing factor 3b subunit 1 (SF3B1), zinc finger (CCCH type), RNA-binding motif and serine/arginine rich 2 (ZRSR2), RAD21 cohesin complex component (RAD21), stromal antigen 1 (STAG1), stromal antigen 2 (STAG2), structural maintenance of chromosomes 1A (SMC1A), and structural maintenance of chromosomes protein 3 (SMC3).

In certain embodiments, methods provided herein comprise treating lymphoma by administering a pharmaceutical composition comprising hypomethylating agent (e.g., 5-azacytidine or decitabine) in combination with one or more additional agents to a human subject in need thereof. Types of lymphomas include non-Hodgkin lymphoma and Hodgkin's disease. Examples of lymphoma include, but are not limited to, diffuse large B-cell lymphoma, anaplastic large-cell lymphoma, Burkitt lymphoma, lymphoblastic lymphoma, mantle cell lymphoma, peripheral T-cell lymphoma, follicular lymphoma, cutaneous T-cell lymphoma, lymphoplasmacytic lymphoma, marginal zone B-cell lymphoma, MALT lymphoma, small-cell lymphocytic lymphoma, and angioimmunoblastic T-cell lymphoma. In some embodiments, the lymphoma is angioimmunoblastic T-cell lymphoma.

In certain embodiments, methods provided herein comprise treating myelodysplastic syndromes, by administering a pharmaceutical composition comprising hypomethylating agent (e.g., 5-azacytidine or decitabine) in combination with one or more additional agents to a human subject in need thereof. MDS may also be classified by using the Revised International Prognostic Scoring System (IPSS-R), which classifies patients into 1 of 5 groups, from very low risk to very high risk, based on risk of mortality and transformation to acute myeloid leukemia (AML). Higher-risk MDS as used in the disclosure is defined as High or Very High risk according to the Revised International Scoring System (IPSS-R). Greenberg, P. L. et al., Blood 2012 Sep. 20; 120 (12): 2454-2465. The scoring system for the IPSS-R is based on the following factors: the percentage of blasts (very early forms of blood cells) in the bone marrow, the type and number of chromosome abnormalities in the cells, the level of red blood cells (measured as hemoglobin) in the patient's blood, the level of platelets in the patient's blood, and the level of neutrophils (a type of white blood cell) in the patient's blood. Each factor is assigned a score and the total sum of the score is used to assign the MDS patient into one of the following five risk groups: Very low (Risk score of <1.5); Low risk (Risk score of >1.5-3); Intermediate risk (Risk score of >3-4.5); High risk (Risk Score of >4.5-6); and Very high risk (Risk Score of >6). About 13% of MDS patients are classified as High Risk, which has a mean overall survival of 1.6 years while about 10% of MDS patients are classified as Very High Risk, which has a mean overall survival of 0.8 years. In some embodiments, the MDS is MDS that is classified as High Risk or Very High Risk as defined by the IPSS-R.

### Dosing Regimens for Hypomethylating Agent and an Additional Therapeutic Agent

Certain embodiments herein provide methods of treating diseases or disorders provided herein (e.g., diseases or disorders involving abnormal cell proliferation), wherein the methods comprise co-administering an oral formulation disclosed herein (such as, for example, an oral formulation comprising a hypomethylating agent (e.g., 5-azacytidine or decitabine) with one or more additional therapeutic agents (such as, for example, a cancer therapeutic agent) to yield a synergistic therapeutic effect. Particular co-administered therapeutic agents useful in the methods provided herein are provided throughout the specification. In particular embodiments, the additional therapeutic agent is co-administered in an amount that is a therapeutically effective amount. In particular embodiments, the additional therapeutic agent is co-administered in a separate dosage form from hypomethylating agent (e.g., 5-azacytidine or decitabine) dosage form with which it is co-administered. In particular embodiments, the additional therapeutic agent is co-administered in a dosage form (e.g., a single unit dosage form) together with hypomethylating agent (e.g., 5-azacytidine or decitabine) with which it is co-administered. In such cases, the hypomethylating agent (e.g., 5-azacytidine or decitabine) and the additional therapeutic agent may be co-formulated together in the same dosage form using methods of co-formulating active pharmaceutical ingredients, including methods disclosed herein and methods known in the art.

In some embodiments provided herein is a method of treating a human having acute myeloid leukemia, wherein the method includes administering to the human a pharmaceutical composition including hypomethylating agent (e.g., 5-azacytidine or decitabine); and wherein the method further includes administering at least one additional therapeutic agent.

In some embodiments provided herein is a method of treating a human having myelodysplastic syndrome, wherein the method includes administering to the human a pharmaceutical composition including hypomethylating agent (e.g., 5-azacytidine or decitabine);
and wherein the method further includes administering at least one additional therapeutic agent. Examples of additional therapeutic agents include but are not limited to an agent that modulates NPM1 pathway or targets nucleophosmin, a FLT3 inhibitor, an agent or therapy that restore the wild-type DNMT3A function, a PI3K/Akt/mTOR pathway inhibitor, a TP53 inhibitor, an agent that targets spliceosome or its downstream target(s), an IDH2 inhibitor, or a RAS pathway inhibitor, as provided herein.

In one embodiment provided herein, the pharmaceutical composition that includes hypomethylating agent (e.g., 5-azacytidine or decitabine) is administered orally.

In one embodiment provided herein, the pharmaceutical composition including hypomethylating agent (e.g., 5-azacytidine or decitabine) is a capsule.

In one embodiment provided herein, the pharmaceutical composition including hypomethylating agent (e.g., 5-azacytidine or decitabine) is a tablet.

In some embodiments, the hypomethylating agent (e.g., 5-azacytidine or decitabine) and the at least one additional therapeutic agent are administered concomitantly. In some embodiments, the hypomethylating agent (e.g., 5-azacytidine or decitabine) and the at least one additional therapeutic agent are administered sequentially, wherein the hypomethylating agent (e.g., 5-azacytidine or decitabine) is administered first. In some embodiments, the hypomethylating agent (e.g., 5-azacytidine or decitabine) and the at least one additional therapeutic agent are co-formulated as a single unit dosage form. In some embodiments, the additional therapeutic agent is administered parenterally. In some embodiments, the additional therapeutic agent is administered orally.

In some embodiments, the hypomethylating agent (e.g., 5-azacytidine or decitabine) is administered orally. In some embodiments, the hypomethylating agent (e.g., 5-azacytidine or decitabine) is administered at a dose of about 50 mg, about 60 mg, about 70 mg, about 80 mg, about 90 mg, about 100 mg, about 150 mg, about 200 mg, about 250 mg, about 300 mg, about 350 mg, about 400 mg, about 450 mg, about 500 mg, about 550 mg, or 600 mg orally. In some embodiments, the hypomethylating agent (e.g., 5-azacytidine or decitabine) is administered at a dose of about 200 mg. In some embodiments, the hypomethylating agent (e.g., 5-azacytidine or decitabine) is administered at a dose of about 300 mg. In some embodiments, the hypomethylating agent (e.g., 5-azacytidine or decitabine) is administered orally for the first seven, fourteen, or twenty-one days of a cycle. In some embodiments, the hypomethylating agent (e.g., 5-azacytidine or decitabine) is administered orally for the first seven, fourteen, or twenty-one days in a 28-day cycle. In some embodiments, the hypomethylating agent (e.g., 5-azacytidine or decitabine) administered to the human subject once or two times per day. In some embodiments, the hypomethylating agent (e.g., 5-azacytidine or decitabine) is administered in the form of a capsule or a tablet. In some embodiments, the tablet is a non-enteric coated tablet.

In some embodiments, the hypomethylating agent (e.g., 5-azacytidine or decitabine) and the at least one additional therapeutic agent provides a synergistic effect to treat the diseases disclosed herein. Synergy may be measured by using the highest single agent (HSA) model and Combenefit package (Di Veroli et al., Bioinformatics. 2016 Sep. 15; 32(18):2866-8.) Specifically, the following are used to determine the synergistic interactions between two drugs: (a) a demonstration of shift in dose response curves determined from their EC₅₀ (i.e., a potency shift) and/or an augmentation of the maximal inhibitory effect; (b) response surface analyses (enabled the visualization of synergy, additivity or antagonism over a matrix of concentration between the two drugs); and (c) the combination index score (derived using a software application Combenefit). The limit of where the synergy index becomes significant is determined empirically and is based on the variance in the data and a confirmation in a potency shift in ECSo. In other words, a combination index, without the clear shift in a dose response curves would not constitute a synergistic interaction. As used herein, the synergistic effect is defined as having EC₅₀ shift at about greater than 4 and/or a synergy index of greater than about 20 as measured by the HSA model and Combenefit package. A negative cell line is used as a control to set the relative “threshold.” In other words, the EC₅₀ and maximal inhibitory effect from the negative control cell line provide baseline potency results, and the shift in EC₅₀ and maximal inhibitory effect of the drug combination is compared to the results from the negative control cell line to determine whether there the drug combination provided a synergistic effect.

In some embodiments, the therapeutic effect of (1) the hypomethylating agent (e.g., 5-azacytidine or decitabine) administered orally and at least one additional therapeutic agent is better than the therapeutic effect of (2) the hypomethylating agent (e.g., 5-azacytidine or decitabine) alone, (3) the at least one additional therapeutic alone, and/or (4) the combination of the hypomethylating agent (e.g., 5-azacytidine or decitabine) administered intravenously or subcutaneously and at the at least one additional therapeutic agent.

In some embodiments, the hypomethylating agent (e.g., 5-azacytidine or decitabine) and at least one additional therapeutic agent increases median survival as compared to the hypomethylating agent (e.g., 5-azacytidine or decitabine) alone. In some embodiments, the hypomethylating agent (e.g., 5-azacytidine or decitabine) and at least one additional therapeutic agent increases median survival as compared to the hypomethylating agent (e.g., 5-azacytidine or decitabine) alone by about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, as measured by any clinically recognized technique.

In some embodiments, the hypomethylating agent (e.g., 5-azacytidine or decitabine) and at least one additional therapeutic agent increases median survival as compared to at least one additional therapeutic agent alone. In some embodiments, the hypomethylating agent (e.g., 5-azacytidine or decitabine) and at least one additional therapeutic agent increases median survival as compared to at least one additional therapeutic agent alone by about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, as measured by any clinically recognized technique.

In some embodiments, the hypomethylating agent (e.g., 5-azacytidine or decitabine) and at least one additional therapeutic agent increases median survival as compared to hypomethylating agent (e.g., 5-azacytidine or decitabine) administered intravenously or subcutaneously and at least one additional therapeutic agent. In some embodiments, the hypomethylating agent (e.g., 5-azacytidine or decitabine) and at least one additional therapeutic agent increases median survival as compared to hypomethylating agent (e.g., 5-azacytidine or decitabine) administered intravenously or subcutaneously and at least one additional therapeutic agent by about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, as measured by any clinically recognized technique.

In one embodiment provided herein, the method includes: (i) administering the hypomethylating agent (e.g., 5-azacytidine or decitabine) to the human subject for 1, 2, or 3 days; and (ii) administering the at least one additional therapeutic agent to the human subject for one or more days. In one embodiment provided herein, the method further includes repeating steps (i) and (ii).

In one embodiment provided herein, the method includes: (i) administering the hypomethylating agent (e.g., 5-azacytidine or decitabine) daily to the human subject for 1, 2, 3, 4, 5, 6, 7, 8, 9, 10, 11, 12, 13, or 14 days; (ii) administering the at least one additional therapeutic agent to the human subject for one or more days; and (iii) optionally repeating steps (i) and (ii).

In one embodiment provided herein, the method includes: (i) administering the hypomethylating agent (e.g., 5-azacytidine or decitabine) daily to the human subject for 1, 2, 3, 4, 5, 6, 7, 8, 9, 10, 11, 12, 13, 14, 15, 16, 17, 18, 19, 20, or 21 days; and (ii) administering the at least one additional therapeutic agent to the human subject for one or more days. In one embodiment provided herein, the method further includes repeating steps (i) and (ii).

In one embodiment provided herein, the method includes: (i) administering the hypomethylating agent (e.g., 5-azacytidine or decitabine) daily to the human subject for 1, 2, 3, 4, 5, 6, 7, 8, 9, 10, 11, 12, 13, or 14 days of a 28-day cycle; (ii) concurrently administering the at least one additional therapeutic agent daily to the human subject for 1, 2, 3, 4, 5, 6, 7, 8, 9, 10, 11, 12, 13, 14, 15, 16, 17, 18, 19, 20, 21, 22, 23, 24, 25, 26, 27, or 28 days of a 28-day cycle; and (iii) optionally repeating steps (i) and (ii).

In one embodiment provided herein, the method includes: (i) administering the hypomethylating agent (e.g., 5-azacytidine or decitabine) daily to the human subject for 1, 2, 3, 4, 5, 6, 7, 8, 9, 10, 11, 12, 13, 14, 15, 16, 17, 18, 19, 20, or 21 days of a 28-day cycle; (ii) concurrently administering the at least one additional therapeutic agent daily to the human subject for 1, 2, 3, 4, 5, 6, 7, 8, 9, 10, 11, 12, 13, 14, 15, 16, 17, 18, 19, 20, 21, 22, 23, 24, 25, 26, 27, or 28 days of a 28-day cycle; and (iii) optionally repeating steps (i) and (ii).

In one embodiment provided herein, the method includes the sequential steps of: (i) administering the hypomethylating agent (e.g., 5-azacytidine or decitabine) to the human subject for 7 days of a 28-day cycle; (ii) administering the at least one additional therapeutic agent to the human subject for 1 day of a 28-day cycle; (iii) administering the hypomethylating agent (e.g., 5-azacytidine or decitabine) to the human subject for 6 days of a 28-day cycle; and (iv) repeating steps (i) to (iii) after 7 days of a resting period.

In one embodiment provided herein, the method includes the sequential steps of: (i) administering the hypomethylating agent (e.g., 5-azacytidine or decitabine) daily to the human subject for 1, 2, 3, 4, 5, 6, 7, 8, 9, 10, 11, 12, 13, 14, 15, 16, 17, 18, 19, 20, or 21 days of a 28-day cycle; (ii) administering the at least one additional therapeutic agent daily to the human subject for 1, 2, 3, 4, 5, 6, 7, 8, 9, 10, 11, 12, 13, 14, 15, 16, 17, 18, 19, 20, or 21 days of a 28-day cycle; (iii) administering the hypomethylating agent (e.g., 5-azacytidine or decitabine) daily to the human subject for 1, 2, 3, 4, 5, 6, 7, 8, 9, 10, 11, 12, 13, 14, 15, 16, 17, 18, 19, 20, or 21 days of a 28-day cycle; and (iv) repeating steps (i) to (iii) after 1, 2, 3, 4, 5, 6, 7, 8, 9, 10, 11, 12, 13, 14, 15, 16, 17, 18, 19, 20, or 21 days of a resting period.

In one embodiment provided herein, the pharmaceutical composition of hypomethylating agent (e.g., 5-azacytidine or decitabine) includes about 50 mg, about 75 mg, about 100 mg, about 100 mg, about 200 mg, about 250 mg, about 300 mg, about 350 mg, about 360 mg, about 370 mg, about 400 mg, about 470 mg, about 480 mg, about 490 mg, about 500 mg, about 550 mg, or about 600 mg of hypomethylating agent (e.g., 5-azacytidine or decitabine).

In one embodiment, the hypomethylating agent (e.g., 5-azacytidine or decitabine) and at least one additional therapeutic agent or therapy increases median survival as compared to hypomethylating agent (e.g., 5-azacytidine or decitabine) administered intravenously or subcutaneously and at least one additional therapeutic agent or therapy. In one embodiment, the hypomethylating agent (e.g., 5-azacytidine or decitabine) and at least one additional therapeutic agent or therapy increases median survival as compared to hypomethylating agent (e.g., 5-azacytidine or decitabine) administered intravenously or subcutaneously and at least one additional therapeutic agent or therapy by about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

In one embodiment, the method comprises: (a) administering the hypomethylating agent (e.g., 5-azacytidine or decitabine) daily to the human subject for 1, 2, or 3 days; (b) administering the at least one additional therapeutic agent or therapy to the human subject for one or more days; and (c) optionally repeating steps (a) and (b).

In one embodiment, the method comprises: (a) administering the hypomethylating agent (e.g., 5-azacytidine or decitabine) daily to the human subject for 1, 2, 3, 4, 5, 6, 7, 8, 9, 10, 11, 12, 13, or 14 days; (b) administering the at least one additional therapeutic agent or therapy to the human subject for one or more days; and (c) optionally repeating steps (a) and (b).

In one embodiment, the method comprises: (a) administering the hypomethylating agent (e.g., 5-azacytidine or decitabine) daily to the human subject for 1, 2, 3, 4, 5, 6, 7, 8, 9, 10, 11, 12, 13, 14, 15, 16, 17, 18, 19, 20, or 21 days; (b) administering the at least one additional therapeutic agent or therapy to the human subject for one or more days; and (c) optionally repeating steps (a) and (b).

In one embodiment, the method comprises: (a) administering the hypomethylating agent (e.g., 5-azacytidine or decitabine) daily to the human subject for 1, 2, 3, 4, 5, 6, 7, 8, 9, 10, 11, 12, 13, or 14 days of a 28-day cycle; (b) concurrently administering the at least one therapeutic agent or therapy daily to the human subject for 1, 2, 3, 4, 5, 6, 7, 8, 9, 10, 11, 12, 13, 14, 15, 16, 17, 18, 19, 20, 21, 22, 23, 24, 25, 26, 27, or 28 days of a 28-day cycle; and (c) optionally repeating steps (a) and (b).

In one embodiment, the method comprises: (a) administering the hypomethylating agent (e.g., 5-azacytidine or decitabine) daily to the human subject for 1, 2, 3, 4, 5, 6, 7, 8, 9, 10, 11, 12, 13, 14, 15, 16, 17, 18, 19, 20, or 21 days of a 28-day cycle; (b) concurrently administering the at least one additional therapeutic agent or therapy daily to the human subject for 1, 2, 3, 4, 5, 6, 7, 8, 9, 10, 11, 12, 13, 14, 15, 16, 17, 18, 19, 20, 21, 22, 23, 24, 25, 26, 27, or 28 days of a 28-day cycle; and (c) optionally repeating steps (a) and (b).

In one embodiment, the method comprises the sequential steps of: (a) administering the hypomethylating agent (e.g., 5-azacytidine or decitabine) to the human subject for 7 days of a 28-day cycle; (b) administering the at least one additional therapeutic agent or therapy to the human subject for 1 day of a 28-day cycle; (c) administering the hypomethylating agent (e.g., 5-azacytidine or decitabine) to the human subject for 6 days of a 28-day cycle; and (d) repeating steps (a) to (c) after 7 days of a resting period.

In one embodiment, the method comprises the sequential steps of: (a) administering the hypomethylating agent (e.g., 5-azacytidine or decitabine) daily to the human subject for 1, 2, 3, 4, 5, 6, 7, 8, 9, 10, 11, 12, 13, 14, 15, 16, 17, 18, 19, 20, or 21 days of a 28-day cycle; (b) administering the at least one additional therapeutic agent or therapy to the human subject daily for 1, 2, 3, 4, 5, 6, 7, 8, 9, 10, 11, 12, 13, 14, 15, 16, 17, 18, 19, 20, or 21 days of a 28-day cycle;
(c) administering the hypomethylating agent (e.g., 5-azacytidine or decitabine) to the human subject daily for 1, 2, 3, 4, 5, 6, 7, 8, 9, 10, 11, 12, 13, 14, 15, 16, 17, 18, 19, 20, or 21 days of a 28-day cycle; and (d) optionally repeating steps (a) and (c) after 1, 2, 3, 4, 5, 6, 7, 8, 9, 10, 11, 12, 13, 14, 15, 16, 17, 18, 19, 20, or 21 days of a resting period.

In one embodiment provided herein, the at least one additional therapeutic agent is administered parenterally.

In one embodiment provided herein, the at least one additional therapeutic agent is administered orally.

### Design of Certain Dosage Forms Provided Herein

Provided herein are dosage forms designed to maximize the absorption and/or efficacious delivery of a hypomethylating agent (e.g., 5-azacytidine or decitabine), upon oral administration, e.g., for release substantially in the stomach. Accordingly, certain embodiments herein provide a solid oral dosage form of a hypomethylating agent (e.g., 5-azacytidine or decitabine), using pharmaceutical excipients that effect immediate release of the API upon oral administration, e.g., substantially in the stomach. Particular immediate release formulations comprise a specific amount of a hypomethylating agent (e.g., 5-azacytidine or decitabine) and optionally one or more excipients. In certain embodiments, the formulation is an immediate release tablet or an immediate release capsule (such as, e.g., an HPMC capsule).

Provided herein are methods of making the formulations provided herein including a hypomethylating agent (e.g., 5-azacytidine or decitabine) provided herein (e.g., immediate release oral formulations and/or formulations that release the API substantially in the stomach). In particular embodiments, the formulations provided herein are prepared using conventional methods known to those skilled in the field of pharmaceutical formulation, as described, e.g., in pertinent textbooks. See, e.g., REMINGTON, THE SCIENCE AND PRACTICE OF PHARMACY, 20th Edition, Lippincott Williams & Wilkins, (2000); ANSEL et al., PHARMACEUTICAL DOSAGE FORMS AND DRUG DELIVERY SYSTEMS, 7th Edition, Lippincott Williams & Wilkins, (1999); GIBSON, PHARMACEUTICAL PREFORMULATION AND FORMULATION, CRC Press (2001).

In certain embodiments, the formulation is a tablet, wherein the tablet is manufactured using standard, art-recognized tablet processing procedures and equipment. In certain embodiments, the method for forming the tablets is direct compression of a powdered, crystalline and/or granular composition including a hypomethylating agent (e.g., 5-azacytidine or decitabine), alone or in combination with one or more excipients, such as, for example, carriers, additives, polymers, or the like. In certain embodiments, as an alternative to direct compression, the tablets are prepared using wet granulation or dry granulation processes. In certain embodiments, the tablets are molded rather than compressed, starting with a moist or otherwise tractable material. In certain embodiments, compression and granulation techniques are used.

In certain embodiments, the compressed tablet of a hypomethylating agent (e.g., 5-azacytidine or decitabine) is film-coated. In some embodiments, the film-coated tablets are compressed tablets coated with a thin layer of a polymer capable of forming a skin-like film over the tablet. The film is usually colored and has the advantage to be more durable, less bulky, and less time-consuming to apply. By its composition, the coating may be designed to rupture and expose the core tablet at the desired location within the gastrointestinal tract. The film-coating process, which places a thin skin-tight coating of a plastic-like material over the compressed tablet, may produce coated tablets having essentially the same weight, shape, and size as the originally compressed tablet. In some embodiments, the film-coating is colored to make the tablets attractive and distinctive. In some embodiments, the film-coating solutions are non-aqueous or aqueous. In particular embodiments, the non-aqueous solutions are optionally contain one or more of the following types of materials to provide the desired coating to the tablets: (1) a film former capable of producing smooth, thin films reproducible under conventional coating conditions and applicable to a variety of tablet shapes, such as, for example, cellulose acetate phthalate; (2) an alloying substance providing water solubility or permeability to the film to ensure penetration by body fluids and therapeutic availability of the drug, such as, for example, polyethylene glycol; (3) a plasticizer to produce flexibility and elasticity of the coating and thus provide durability, such as, for example, castor oil; (4) a surfactant to enhance spreadability of the film during application, such as, for example, polyoxyethylene sorbitan derivatives; (5) opaquants and colorants to make the appearance of the coated tablets attractive and distinctive, such as, for example, titanium dioxide as an opaquant, and FD&C or D&C dyes as a colorant; (6) sweeteners, flavors, or aromas to enhance the acceptability of the tablet to the subject, such as, for example, saccharin as sweeteners, and vanillin as flavors and aromas; (7) a glossant to provide a luster to the tablets without a separate polishing operation, such as, for example, beeswax; and (8) a volatile solvent to allow the spread of the other components over the tablets while allowing rapid evaporation to permit an effective yet speedy operation, such as, for example, alcohol-acetone mixture. In certain embodiments, an aqueous film-coating formulation contains one or more of the following: (1) film-forming polymer, such as, for example, cellulose ether polymers as hydroxypropyl methyl-cellulose, hydroxypropyl cellulose, and methyl-cellulose; (2) plasticizer, such as, for example, glycerin, propylene glycol, polyethylene glycol, diethyl phthalate, and dibutyl subacetate; (3) colorant and opacifier, such as, for example, FD&C or D&C lakes and iron oxide pigments; or (4) vehicle, such as, for example, water.

In certain embodiments, the pharmaceutical formulation is an immediate release tablet of a hypomethylating agent (e.g., 5-azacytidine or decitabine). In certain embodiments, the immediate release tablet is designed, e.g., to disintegrate and release the API absent of any special rate-controlling features, such as special coatings and other techniques.

In certain embodiments, the pharmaceutical formulations provided herein contain a hypomethylating agent (e.g., 5-azacytidine or decitabine) and, optionally, one or more excipients to form a “drug core.” Optional excipients include, e.g., diluents (bulking agents), lubricants, disintegrants, fillers, stabilizers, surfactants, preservatives, coloring agents, flavoring agents, binding agents, excipient supports, glidants, permeation enhancement excipients, plasticizers and the like, e.g., as known in the art.

One or more diluents may be used, e.g., to increase bulk so that a practical size tablet is ultimately provided. Diluents also include, e.g., ammonium alginate, calcium carbonate, calcium phosphate, calcium sulfate, cellulose acetate, compressible sugar, confectioner's sugar, dextrates, dextrin, dextrose, erythritol, ethylcellulose, fructose, fumaric acid, glyceryl palmitostearate, isomalt, kaolin, lacitol, lactose, mannitol, magnesium carbonate, magnesium oxide, maltodextrin, maltose, medium-chain triglycerides, microcrystalline cellulose, microcrystalline silicified cellulose, powered cellulose, polydextrose, polymethylacrylates, simethicone, sodium alginate, sodium chloride, sorbitol, starch, pregelatinized starch, sucrose, sulfobutylether-fP-cyclodextrin, talc, tragacanth, trehalose, and xylitol. In some embodiments, the diluents comprise mannitol and microcrystalline silicified cellulose. Diluents may be used in amounts calculated to obtain a desired volume for a tablet. In some embodiments, a diluent is used in an amount of about 5% or more, about 10% or more, about 15% or more, about 20% or more, about 22% or more, about 24% or more, about 26% or more, about 28% or more, about 30% or more, about 32% or more, about 34% or more, about 36% or more, about 38% or more, about 40% or more, about 42% or more, about 44% or more, about 46% or more, about 48% or more, about 50% or more. In some embodiments, a diluent used in the formulation is between about 20% and about 40% w/w of the drug core.

One or more lubricants may be used, e.g., to facilitate tablet manufacture. Examples of suitable lubricants include, for example, vegetable oils such as peanut oil, cottonseed oil, sesame oil, olive oil, corn oil, and oil of theobroma, glycerin, magnesium stearate, calcium stearate, and stearic acid. In certain embodiments, stearates, if present, represent no more than approximately 2 weight % of the drug-containing core. In particular embodiments, the lubricant is magnesium stearate. In certain embodiments, the lubricant is present, relative to the drug core, in an amount of about 0.2% w/w of the drug core, about 0.4% w/w of the drug core, about 0.6% w/w of the drug core, about 0.8% w/w of the drug core, about 1.0% w/w of the drug core, about 1.2% w/w of the drug core, about 1.4% w/w of the drug core, about 1.6% w/w of the drug core, about 1.8% w/w of the drug core, about 2.0% w/w of the drug core, about 2.2% w/w of the drug core, about 2.4% w/w of the drug core, about 2.6% w/w of the drug core, about 2.8% w/w of the drug core, about 3.0% w/w of the drug core, about 3.5% w/w of the drug core, about 4% w/w of the drug core, about 4.5% w/w of the drug core, or about 5% w/w of the drug core. In some embodiments, the lubricant is present in an amount of between about 0.5% and about 5% w/w of the drug core, or between about 1% and about 3% w/w of the drug core.

One or more disintegrants may be used, e.g., to facilitate disintegration of the tablet, and may be, e.g., starches, clays, celluloses, algins, gums or crosslinked polymers. Disintegrants also include, e.g., alginic acid, carboxymethylcellulose calcium, carboxymethylcellulose sodium (e.g., AC-DI-SOL, PRIMELLOSE), colloidal silicon dioxide, croscarmellose sodium, crospovidone (e.g., KOLLIDON, POLYPLASDONE), guar gum, magnesium aluminum silicate, methyl cellulose, microcrystalline cellulose, polacrilin potassium, powdered cellulose, pregelatinized starch, sodium alginate, sodium starch glycolate (e.g., EXPLOTAB) and starch. In some embodiments, the disintegrant is croscarmellose sodium. In certain embodiments, the disintegrant is, relative to the drug core, present in the amount of about 1% w/w of the drug core, about 2% w/w of the drug core, about 3% w/w of the drug core, about 4% w/w of the drug core, about 5% w/w of the drug core, about 6% w/w of the drug core, about 7% w/w of the drug core, about 8% w/w of the drug core, about 9% w/w of the drug core, or about 10% w/w of the drug core. In some embodiments, the disintegrant is present in the amount of about between about 1% and about 10% w/w of the drug core, between about 2% and about 8% w/w of the drug core.

### 5-Azacytidine

In certain embodiments, the hypomethylating agent used in the methods provided here is 5-azacytidine.

### A. Compound

5-Azacytidine (National Service Center designation NSC-102816; CAS Registry Number 320-67-2) is also known as azacitidine, abbreviated as AZA, or 4-amino-l-B-D-ribofuranosyl-1,3,5-triazin-2(1H)-one. The marketed product VIDAZA© (5-azacytidine for injection) contains 5-azacytidine, and is for subcutaneous or intravenous use. The marketed product ONUREG©also contains 5-azacytidine, and is in the form of tablets for oral administration. 5-Azacytidine is a pyrimidine nucleoside analog of cytidine. 5-Azacytidine has the following structure:

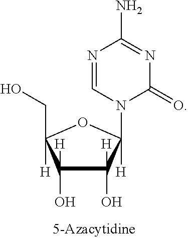

After its incorporation into replicating DNA, 5-azacytidine forms a covalent complex with DNA methyltransferases. DNA methyltransferases are responsible for de novo DNA methylation and for reproducing established methylation patterns in daughter DNA strands of replicating DNA. Inhibition of DNA methyltransferases by 5-azacytidine leads to DNA hypomethylation, thereby restoring normal functions to morphologically dysplastic, immature hematopoietic cells and cancer cells by re-expression of genes involved in normal cell cycle regulation, differentiation and death. The cytotoxic effects of these cytidine analogs cause the death of rapidly dividing cells, including cancer cells, that are no longer responsive to normal cell growth control mechanisms. 5-azacytidine also incorporates into RNA. The cytotoxic effects of 5-azacytidine may result from multiple mechanisms, including inhibition of DNA, RNA and protein synthesis, incorporation into RNA and DNA, and activation of DNA damage pathways.

Injectable 5-azacytidine has been tested in clinical trials and showed significant anti-tumor activity, such as, for example, in the treatment of myelodysplastic syndromes (MDS), acute myelogenous leukemia (AML), chronic myelogenous leukemia (CML), acute lymphocytic leukemia (ALL), and non Hodgkin's lymphoma (NHL). See, e.g., Aparicio et al., Curr. Opin. Invest. Drugs 3(4): 627-33 (2002).

5-Azacytidine is approved for subcutaneous (SC) or intravenous (IV) administration to treat patients with the following French-American-British (FAB) myelodysplastic syndrome subtypes: refractory anemia (RA) or refractory anemia with ringed sideroblasts (if accompanied by neutropenia or thrombocytopenia or requiring transfusions), refractory anemia with excess blasts (RAEB), refractory anemia with excess blasts in transformation (RAEB-T), and chronic myelomonocytic leukemia (CMMoL). Oral dosing has been studied in clinical trials, such as NCT00761722, NCT01519011, NCT00528982, and NCT01757535. 5-Azacytidine is approved for oral administration for continued treatment of adult patients with acute myeloid leukemia who achieved first complete remission (CR) or complete remission with incomplete blood count recovery (CRi) following intensive induction chemotherapy and are not able to complete intensive curative therapy. Oral formulations and methods of treatment using oral 5-azacytidine are disclosed in U.S. Pat. No. 8,846,628, which is incorporated by reference in its entirety.

In some embodiments, 5-azacytidine is administered orally. In some embodiments, 5-azacytidine is administered in the form of a capsule or a tablet. In some embodiments, the tablet is a non-enteric-coated tablet. In some embodiments, the 5-azacytidine is administered at a dose of about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg or about 600 mg orally. In some embodiments, the 5-azacytidine is administered at a dose of about 200 to about 300 mg orally. In some embodiments, the 5-azacytidine is administered at a dose of about 200 mg. In some embodiments, the 5-azacytidine is administered at a dose of about 300 mg. In some embodiments, the 5-azacytidine is administered at a dose of 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg, 400 mg, 410 mg, 420 mg, 430 mg, 440 mg, 450 mg, 460 mg, 470 mg, 480 mg, 490 mg, 500 mg, 510 mg, 520 mg, 530 mg, 540 mg, 550 mg, 560 mg, 570 mg, 580 mg or 600 mg orally. In some embodiments, the 5-azacytidine is administered at a dose of 200 to 300 mg orally. In some embodiments, the 5-azacytidine is administered at a dose of 200 mg. In some embodiments, the 5-azacytidine is administered at a dose of 300 mg. In some embodiments, 5-azacytidine is administered daily orally for the first seven, fourteen, or twenty-one days of a 28-day cycle. In some embodiments, 5-azacytidine is administered daily orally for the first fourteen days of a 28-day cycle. In some embodiments, 5-azacytidine administered to the human subject once per day. In some embodiments, 5-azacytidine administered to the human subject two times per day.

In some embodiments, the 5-azacytidine is administered orally at a dose of about 200 mg per day for 14 days in a 28-day cycle. In some embodiments, the 5-azacytidine is administered orally at a dose of 200 mg per day for 14 days in a 28-day cycle. In some embodiments, the 5-azacytidine is administered orally at a dose of about 300 mg per day for 14 days in a 28-day cycle. In some embodiments, the 5-azacytidine is administered orally at a dose of 300 mg per day for 14 days in a 28-day cycle. In some embodiments, the 5-azacytidine is administered orally at a dose of about 200 mg per day for 21 days in a 28-day cycle. In some embodiments, the 5-azacytidine is administered orally at a dose of 200 mg per day for 21 days in a 28-day cycle. In some embodiments, the 5-azacytidine is administered orally at a dose of about 300 mg per day for 21 days in a 28-day cycle. In some embodiments, the 5-azacytidine is administered orally at a dose of 300 mg per day for 21 days in a 28-day cycle.

In some embodiments, the 5-azacytidine is administered orally daily for 1, 2, 3, 4, 5, 6, 7, 8, 9, 10, 11, 12, 13, 14, or greater than 14 days, optionally followed by a treatment dosing holiday of 1, 2, 3, 4, 5, 6, 7, 8, 9, 10, 11, 12, 13, 14, or greater than 14 days. In some embodiments, the 5-azacytidine is administered orally daily for 14 or more days, optionally followed by a treatment dosing holiday of 7 or more days. In some embodiments, the 5-azacytidine is administered orally for 21 or more days, optionally followed by a treatment dosing holiday of 7 or more days. In some embodiments, the 5-azacytidine is administered orally for 14 days, optionally followed by a treatment dosing holiday of 14 days. In some embodiments, the 5-azacytidine is administered orally for 21 or more days, followed by a treatment dosing holiday of 7 or more days. In some embodiments, the 5-azacytidine is administered orally for 14 days, followed by a treatment dosing holiday of 14 days.

In some embodiments, the 5-azacytidine is administered orally at a dose of about 300 mg daily for 14 days, followed by a treatment dosing holiday of 14 days. In some embodiments, the 5-azacytidine is administered orally at a dose of 300 mg daily for 14 days, followed by a treatment dosing holiday of 14 days. In some embodiments, the 5-azacytidine is administered orally at a dose of about 200 mg daily for 14 days, followed by a treatment dosing holiday of 14 days. In some embodiments, the 5-azacytidine is administered orally at a dose of 200 mg daily for 14 days, followed by a treatment dosing holiday of 14 days. In some embodiments, the 5-azacytidine is administered orally at a dose of about 300 mg daily for 21 days, followed by a treatment dosing holiday of 7 days. In some embodiments, the 5-azacytidine is administered orally at a dose of 300 mg daily for 21 days, followed by a treatment dosing holiday of 7 days. In some embodiments, the 5-azacytidine is administered orally at a dose of about 200 mg daily, followed by a treatment dosing holiday of 7 days. In some embodiments, the 5-azacytidine is administered orally at a dose of 200 mg daily, followed by a treatment dosing holiday of 7 days.

In some embodiments, the 5-azacytidine is administered orally using a treatment cycle comprising administration of 5-azacytidine per day for 7 days in a 28-day cycle. In some embodiments, the 5-azacytidine is administered orally using a treatment cycle comprising administration of 5-azacytidine per day for 14 days in a 28-day cycle. In some embodiments, the 5-azacytidine is administered orally using a treatment cycle comprising administration of 5-azacytidine per day for 21 days in a 28-day cycle.

5-azacytidine exerts effects on cell viability and epigenetic reprogramming of cells. Taylor and Jones, Cell 20(1):85-93 (1980). At high doses, 5-azacytidine is thought to exercise a predominantly acute cytotoxic effect (Khan et al., Experimental Hematology 36(2): 149-57, 2008), while at low doses it inhibits clonogenicity of tumor cells though differentiation (Tsai et al., Cancer Cell, 21(3): 430-46, 2012).

The marketed product VIDAZA®, the injectable formulation of 5-azacytidine, is administered at relatively higher doses and for shorter duration compared to the oral, non-enteric coated formulation of 5-azacytidine as described in U.S. Pat. No. 8,846,628, including CC-486. As used herein and unless otherwise specified, the term “CC-486” or ONUREG® refers to 5-azacytidine for oral use. Clinical studies revealed that CC-486 induces more sustainable demethylative effects as compared to VIDAZA® (Laille et al., PLOSOne 10(8):e0135520, 2015), although cumulative exposures of 14 or 21 day regimens of CC-486 are lower than VIDAZA®, administered for 5 days (Garcia-Manero et al., Leukemia 30(4):889-96, 2016).

To examine differences in cytotoxic and epigenetic effects as a function of duration of exposure to 5-azacytidine, high exposure, limited duration (HELD) administration of injectable 5-azacytidine was compared with low exposure, extended duration (LEED) administration of 5-azacytidine administered orally. To model injectable and oral azacytidine dosing in non-clinical systems, the total 5-azacytidine exposure was held constant while varying the duration of exposure. In some embodiments, to model the oral administration of 5-azacytidine, the 5-azacytidine was delivered at a low exposure for extended duration (LEED), at a dose of 1 mg/kg, once daily for fifteen days (QDx15). To model the same cumulative dose by intravenous or subcutaneous administration of 5-azacytidine, the 5-azacytidine was administered at a high exposure for a limited duration (HELD), at a dose of 3 mg/kg, once daily for five days (QDx5).

In some embodiments, LEED administration of 5-azacytidine in combination with other agents provides a sustained pharmacodynamic effect and/or improved patient compliance. A sustained pharmacodynamic effect may include any change elicited by 5-azacytidine, which includes for example MCL-1 degradation, and/or changes in ATF3 or SCD gene expression. In some embodiments, LEED of 5-azacyitidine in combination with other agents provides a reduction in global DNA methylation (e.g., due to increased nucleic acid incorporation) that sustained through the end of the treatment cycle (i.e., a 28-day cycle) compared to HELD of 5-azacyitidine in combination with other agents. In some embodiments, LEED of 5-azacyitidine in combination with other agents provides a differentiation maker upregulation that peaks at Day 21 of a 28-day cycle and has a cell death that is characterized by a gradual loss of viability through Day 28 of a 28-day cycle. In some embodiments, HELD of 5-azacyitidine in combination with other agents provides a differentiation marker upregulation that peaks at Day 7 of a 28-day cycle and has a cell death that is characterized by a peak at Day 14 followed by recovery in a 28-day cycle. In some embodiments, LEED of 5-azacyitidine in combination with other agents provides a higher expression of myeloid differentiation markers, which include but are not limited to CD11b, CD14, CD86, HLA-DR and MERTK, that is sustained through a treatment cycle (i.e., a 28-day cycle) compared to HELD of 5-azacyitidine in combination with other agents. In some embodiments, LEED of 5-azacyitidine in combination with other agents provides more pronounced epigenetic changes and more extensive differentiation compared to HELD of 5-azacyitidine in combination with other agents.

### B. Pharmaceutical Formulations

Embodiments herein encompass pharmaceutical formulations and compositions comprising 5-azacytidine, and optionally a permeation enhancer, wherein the formulations and compositions are prepared for oral administration. In a particular embodiment, the formulations and compositions are prepared for release of 5-azacytidine substantially in the stomach. In specific embodiments, 5-azacytidine and the pharmaceutical formulations and compositions are used for treating diseases and disorders associated with abnormal cell proliferation, wherein 5-azacytidine, the formulations and compositions are prepared for oral administration, preferably for release of 5-azacytidine substantially in the stomach. Particular embodiments relate to the use 5-azacytidine for the preparation of pharmaceutical formulations and compositions for treating particular medical indications, as provided herein. The pharmaceutical formulations and compositions including 5-azacytidine provided herein are intended for oral delivery of 5-azacytidine to subjects in need thereof. Oral delivery formats include, but are not limited to, tablets, capsules, caplets, solutions, suspensions, and syrups.

Particular embodiments herein provide solid oral dosage forms that are tablets or capsules. In certain embodiments, the formulation is a tablet containing 5-azacytidine. In certain embodiments, the formulation is a capsule containing 5-azacytidine. In certain embodiments, the tablets or capsules provided herein optionally comprise one or more excipients, such as, for example, glidants, diluents, lubricants, colorants, disintegrants, granulating agents, binding agents, polymers, and coating agents. In certain embodiments, embodiments herein encompass the use of 5-azacytidine, for the preparation of a pharmaceutical composition for treating a disease associated with abnormal cell proliferation, wherein the composition is prepared for oral administration.

In certain embodiments, the formulations including 5-azacytidine effect an immediate release of the active pharmaceutical ingredient (API) upon oral administration. In particular embodiments, the formulations including 5-azacytidine comprise a therapeutically effective amount of 5-azacytidine (and, optionally, one or more excipients) and effect an immediate release of the API upon oral administration.

In certain embodiments, the formulations including 5-azacytidine release the API substantially in the stomach upon oral administration. In certain embodiments, the formulations effect an immediate release of 5-azacytidine upon oral administration. In certain embodiments, the formulations further comprise a drug release controlling component which is capable of releasing 5-azacytidine substantially in the stomach. In certain embodiments, the formulations optionally further comprises a drug release controlling component, wherein the drug release controlling component is adjusted such that the release of 5-azacytidine occurs substantially in the stomach. In particular embodiments, the drug release controlling component is adjusted such that the release of 5-azacytidine is immediate and occurs substantially in the stomach. In particular embodiments, the drug release controlling component is adjusted such that the release of 5-azacytidine is sustained and occurs substantially in the stomach. In certain embodiments, the formulation of 5-azacytidine releases the API substantially in the stomach, and, subsequently, releases the remainder of the API in the intestine upon oral administration.

Methods by which skilled practitioners can assess the oral bioavailability of a drug formulation in a subject are known in the art. Such methods, include, for example, comparing various pharmacokinetic parameters, such as, but not limited to, maximum plasma concentration (“Cmax”), time to maximum plasma concentration (“Tmax”), or area-under-the-curve (“AUC”) determinations.

Particular embodiments herein provide pharmaceutical formulations (e.g., immediate release oral formulations and/or formulations that release the API substantially in the stomach) including 5-azacytidine that achieve a particular AUC value (e.g., AUC(O-t) or AUC(O-oo)) in the subject (e.g., human) to which the formulation is orally administered. Particular embodiments provide oral formulations that achieve an AUC value of at least 25 ng-hr/mL, at least 50 ng-hr/mL, at least 75 ng-hr/mL, at least 100 ng-hr/mL, at least 150 ng-hr/mL, at least 200 ng-hr/mL, at least 250 ng-hr/mL, at least 300 ng-hr/mL, at least 350 ng-hr/mL, at least 400 ng-hr/mL, at least 450 ng-hr/mL, at least 500 ng-hr/mL, at least 550 ng-hr/mL, at least 600 ng-hr/mL, at least 650 ng-hr/mL, at least 700 ng-hr/mL, at least 750 ng-hr/mL, at least 800 ng-hr/mL, at least 850 ng-hr/mL, at least 900 ng-hr/mL, at least 950 ng-hr/mL, at least 1000 ng-hr/mL, at least 1100 ng-hr/mL, at least 1200 ng-hr/mL, at least 1300 ng-hr/mL, at least 1400 ng-hr/mL, at least 1500 ng-hr/mL, at least 1600 ng-hr/mL, at least 1700 ng-hr/mL, at least 1800 ng-hr/mL, at least 1900 ng-hr/mL, at least 2000 ng-hr/mL, at least 2250 ng-hr/mL, or at least 2500 ng-hr/mL. In particular embodiments, the AUC determination is obtained from a time-concentration pharmacokinetic profile obtained from the blood samples of human patients following dosing.

Particular embodiments herein provide pharmaceutical formulations (e.g., immediate release oral formulations and/or formulations that release the API substantially in the stomach) including 5-azacytidine that achieve a particular maximum plasma concentration (“Cmax”) in the human subject to which the formulation is orally administered. Particular embodiments provide oral formulations that achieve a Cmax of 5-azacytidine of at least 25 ng/mL, at least 50 ng/mL, at least 75 ng/mL, at least 100 ng/mL, at least 150 ng/mL, at least 200 ng/mL, at least 250 ng/mL, at least 300 ng/mL, at least 350 ng/mL, at least 400 ng/mL, at least 450 ng/mL, at least 500 ng/mL, at least 550 ng/mL, at least 600 ng/mL, at least 650 ng/mL, at least 700 ng/mL, at least 750 ng/mL, at least 800 ng/mL, at least 850 ng/mL, at least 900 ng/mL, at least 950 ng/mL, at least 1000 ng/mL, at least 1100 ng/mL, at least 1200 ng/mL, at least 1300 ng/mL, at least 1400 ng/mL, at least 1500 ng/mL, at least 1600 ng/mL, at least 1700 ng/mL, at least 1800 ng/mL, at least 1900 ng/mL, at least 2000 ng/mL, at least 2250 ng/mL, or at least 2500 ng/mL. Particular embodiments herein provide pharmaceutical formulations (e.g., immediate release oral formulations and/or formulations that release the API substantially in the stomach) including 5-azacytidine that achieve a particular time to maximum plasma concentration (“Tmax”) in the human subject to which the formulation is orally administered. Particular embodiments provide oral formulations that achieve a Tmax of 5-azacytidine of less than 10 minutes, less than 15 minutes, less than 20 minutes, less than 25 minutes, less than 30 minutes, less than 35 minutes, less than 40 minutes, less than 45 minutes, less than 50 minutes, less than 55 minutes, less than 60 minutes, less than 65 minutes, less than 70 minutes, less than 75 minutes, less than 80 minutes, less than 85 minutes, less than 90 minutes, less than 95 minutes, less than 100 minutes, less than 105 minutes, less than 110 minutes, less than 115 minutes, less than 120 minutes, less than 130 minutes, less than 140 minutes, less than 150 minutes, less than 160 minutes, less than 170 minutes, less than 180 minutes, less than 190 minutes, less than 200 minutes, less than 210 minutes, less than 220 minutes, less than 230 minutes, or less than 240 minutes In particular embodiments, the Tmax value is measured from the time at which the formulation is orally administered.

### VI. Kits

Provided herein are kits for identifying subjects with cancer or disorders related to abnormal cell proliferation who are likely to be responsive to treatment with a hypomethylating agent.

In one embodiment, provided herein is a kit for identifying a subject having a cancer or a disorder related to abnormal cell proliferation who is likely to be responsive to treatment with a hypomethylating agent.

In one embodiment, provided herein is a kit for treating cancer or a disorder related to abnormal cell proliferation, comprising a means for detecting the presence of one or more gene mutations in the sample from a human subject, wherein the treatment compound is a hypomethylating agent.

In one embodiment, the one or more gene mutations detected by the various kits described herein are mutations in NPM1, FLT3, DNMT3A, P53, IDH2 and/or SRSF2.

In one embodiment, the presence of a NPM1 mutation is detected. In one embodiment, the presence of a FLT3 mutation is detected. In one embodiment, the presence of a NPM1 mutation and a FLT3 mutation is detected.

In one embodiment, the FLT3 mutation is a FMS-like tyrosine kinase-3 internal tandem duplication (FLT3-ITD) or FMS-like tyrosine kinase-3 tyrosine kinase domain (FLT3-TKD) mutation. In one embodiment, the FLT3 mutation is a FLT3-ITD mutation. In one embodiment, the FLT3 mutation is a FLT3-TKD mutation. In one embodiment, the FLT3 mutation is a FLT3-ITD and FLT3-TKD co-mutation.

In one embodiment, the presence of a DNMT3A mutation is detected.

In one embodiment, the presence of a P53 mutation is detected.

In one embodiment, the presence of an IDH2 mutation and a SRSF2 mutation is detected.

In one embodiment, the presence of a RAS mutation is not detected.

In one embodiment, the hypomethylating agent is 5-azacytidine or decitabine.

In one embodiment, the human has acute myeloid leukemia. In one embodiment, the human has myelodysplastic syndrome. In one embodiment, the myelodysplastic syndrome is high and very high risk myelodysplastic syndromes as defined by the Revised International Prognostic Scoring System (IPSS-R).

In one embodiment, the cancer is a hematological cancer. In one embodiment, the hematological cancer is acute myeloid leukemia (AML), acute lymphoblastic leukemia (ALL), chronic myeloid leukemia (CML), chronic lymphocytic leukemia (CLL), non-Hodgkin's lymphoma (NHL), Hodgkin's lymphoma, or multiple myeloma (MM).

In one embodiment, the hematological cancer is acute myeloid leukemia (AML). In one embodiment, the AML is de novo AML. In one embodiment, the AML is secondary to prior myelodysplastic disease (MDS) or chronic myelomonocytic leukemia (CMML). In one embodiment, the AML is relapsed or refractory AML. In one embodiment, the AML has intermediate-risk cytogenetic characteristics as defined according to National Comprehensive Cancer Network 2011 guidelines. In one embodiment, the AML has poor-risk cytogenetic characteristics as defined according to National Comprehensive Cancer Network 2011 guidelines.

Such a kit can employ, for example, a dipstick, a membrane, a chip, a disk, a test strip, a filter, a microsphere, a slide, a multi-well plate, or an optical fiber. The solid support of the kit can be, for example, a plastic, silicon, a metal, a resin, glass, a membrane, a particle, a precipitate, a gel, a polymer, a sheet, a sphere, a polysaccharide, a capillary, a film, a plate, or a slide.

In some embodiments, the kits described herein include reagents for DNA sequencing.

In some embodiments, the kits described herein include primers for PCR amplification of mutant loci. In some embodiments, such kits may include primers for PCR as well as probes for qPCR and enzymes suitable for amplifying nucleic acids (e.g., polymerases such as Taq polymerase). In some embodiments, such kits may include multiple primers and multiple probes. In some embodiments, such kits may include a computer program product embedded on computer readable media for predicting whether a human subject is likely to be responsive to treatment with a hypomethylating agent. In some embodiments, the kits may include a computer program product embedded on a computer readable media along with instructions.

In one embodiment, a kit provided herein comprises a hypomethylating agent described herein. Kits may further comprise additional active agents, including but not limited to those disclosed herein.

Kits provided herein may further comprise devices that are used to administer the active ingredients. Examples of such devices include, but are not limited to, syringes, drip bags, patches, and inhalers.

Kits provided herein may further comprise solid phase supports. Examples of solid phases suitable for carrying out the methods disclosed herein include beads, particles, colloids, single surfaces, tubes, multi-well plates, microtiter plates, slides, membranes, gels, and electrodes. When the solid phase is a particulate material (e.g., a bead), it is, in one embodiment, distributed in the wells of multi-well plates to allow for parallel processing of the solid phase supports.

In one embodiment, the kit further comprises an instruction for using the kit. The kit can be tailored for in-home use, clinical use, or research use.

It is noted that any combination of the above-listed embodiments, for example, with respect to one or more reagents, such as, without limitation, nucleic acid primers, solid support, and the like, are also contemplated in relation to any of the various methods and/or kits provided herein.

### EXAMPLES

Certain embodiments of the invention are illustrated by the following non-limiting example.

### Example 1. Analysis of Gene Mutations from Phase 3 Clinical Trial

### Clinical Study Description and Cohort:

The correlative analyses of gene mutations with clinical indices (overal survival (OS), relapse-free survival (RFS)) were performed in the CC0486-AML-001 study (the QUAZAR® study). This clinical trial was a Phase 3, randomized, double-blind, placebo-controlled trial of the oral formulation of azacitidine (CC-486, a DNMT inhibitor and hypomethylating agent), administered as maintenance therapy in patients with AML who were in first remission after intensive chemotherapy with or without consolidation treatment. In this clinical trial, the key enrollment criteria included newly diagnosed de novo or secondary AML patients, with either intermediate or poor risk (based on the 2012 National Comprehensive Cancer Network (NCCN) classification), were 55 years of age or older, who were in complete remission with or without complete blood count recovery. Patients were also those that were not candidates for hematopoietic stem-cell transplantation. See, ClinicalTrials.gov Identifier: NCT01757535, https://clinicaltrials.gov/ct2/show/NCT01757535, the entirety of which is incorporated herein by reference.

Treatment arms included: CC-486 (300 mg) or placebo once daily for 14 days per 28-day cycle. The primary end point was overall survival (OS). Secondary end points included relapse-free survival (RFS).

### Gene Mutational Analyses:

Bone marrow mononuclear cell (BMMC) DNA was isolated from patients at diagnoses (before consideration for study enrollment) and specific gene mutations (NPM1, FLT3 ITD, FLT3 TKD, CEBPA, MLL, P53, cKIT, NRAS) were determined by investigator report or retrospectively identified by central sequencing for available samples. Cytogenetic risk classification was performed according to the 2012 NCCN Guidelines (source: National Comprehensive Cancer Network Clinical Practice Guidelines in Oncology for Acute Myeloid Leukemia; available at https://www.nccn.org/professionals/physician_gls/PDF/aml.pdf); only intermediate- and poor-risk patients were eligible.

At screening (post-IC/C induction chemotherapy/consolidation, when patients achieved complete remission (CR) or CR with incomplete blood count recovery (CRi), gene mutations were identified by targeted next-generation sequencing (ArcherDX VariantPlex Core Myeloid panel; 37 genes, ArcherDX, Boulder, CO) of DNA isolated from BMMCs. Gene mutational analyses and variants reported were based on the sequence coverages of the 37-gene AML panel. Mutation variants were filtered. Karyotypes from post-IC/C bone marrow samples were determined by MLL (Munich Leukemia Laboratory, Munich, Germany). Abnormal karyotype was defined as one cytogenetic abnormality and complex karyotype was defined as three or more cytogenetic abnormalities in the absence of a WHO designated recurring translocation or inversion; i.e., t(8;21)(q22;q22.1); t(9;11)(p21.3;q23.3); inv(3)(q21.3q26.2) or t(3;3)(q21.3;q26.2); t(6;9)(p23;q34.1); t(v;11;q23.3).

When applicable, statistical analyses were performed using Prism Software (v.8.0.0; GraphPad Software, San Diego, CA) or R statistical computing software environment (v3.6.3). OS and RFS were estimated using Kaplan-Meier methods and P values were calculated using the log-rank test. Cox regression analyses were performed to evaluate the association of gene mutation status and treatment arm with OS and RFS. Gene mutation frequencies and landscape were visualized. Correlative analyses including single gene mutation and co-mutational queries were performed with clinical response. All possible pairings between genes and/or cytogenetic alterations were evaluated for tendency to co-occur or for mutual exclusivity; i.e., a pair of alterations occurring together more frequently or rarely than expected by their individual frequencies, as described by Papaemmanuil et al. (N Engl J Med, 2016). 2×2 contingency tables were generated to test putative associations using Fisher's exact tests and adjusted to control for multiple hypothesis testing. Significant differences between groups were assessed using statistical methods listed in the corresponding Figure footnotes. P-values were considered statistically significant at *p≤0.05, **p≤0.01, or ***p≤0.001.

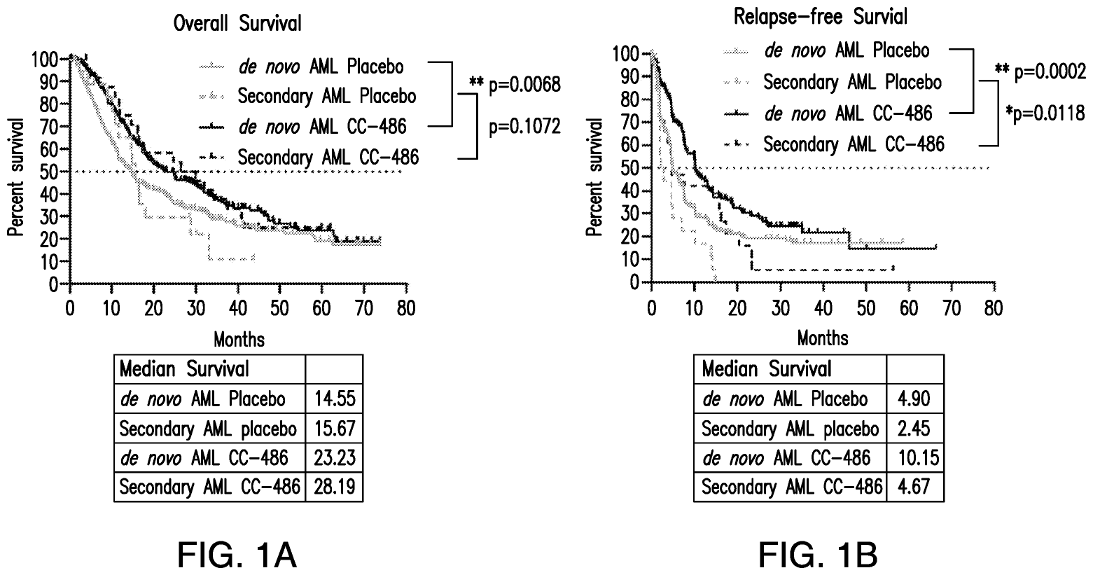

The results of these analyses are shown in FIG. 1 to FIG. 21D, as described in more detail herein.

FIG. 1A and FIG. 1B represent AML subtypes (de novo, n=429; and secondary, n=43) at diagnosis and associations with clinical response. FIG. 1A shows overall survival (OS) for patients in the defined AML subtype and treated with either placebo or CC-486. FIG. 1B shows relapse-free survival (RFS) for patients in the defined AML subtype and treated with either placebo or CC-486. In FIG. 1A, a statistically significant increase in OS was observed comparing de novo AML patients treated with CC-486 versus placebo (median OS 23.23 months versus 14.55 months, respectively; p=0.0068). An increase in OS was observed in secondary AML patients treated with CC-486 versus placebo (median OS 28.19 months versus 15.67 months, respectively; p=0.1072). In FIG. 1B, statistically significant increases in RFS were observed comparing de novo AML patients treated with CC-486 versus placebo (median RFS 10.15 months versus 4.90 months, respectively; p=0.0002), and in secondary AML patients treated with CC-486 versus placebo (median RFS 4.67 months versus 2.45 months, respectively; p=0.0118). Figures were illustrated using Kaplan-Meier analyses and p-values were calculated using log-rank test.

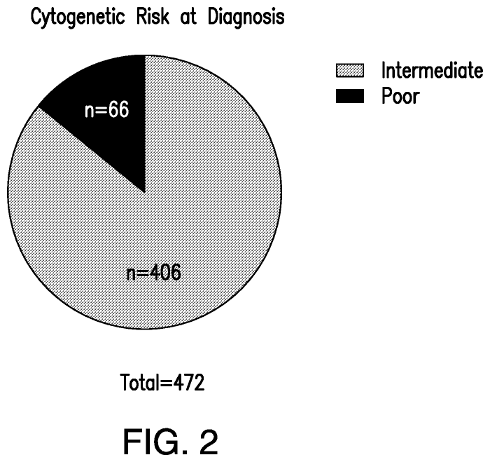

FIG. 2 represents cytogenetic risk (intermediate- or poor-risk) at diagnosis; favorable-risk patients were excluded from the trial. There were 406 out of 472 patients characterized as intermediate-risk and 66 out of 472 patients characterized as poor-risk. Cytogenetic risk classification was performed according to the 2012 National Comprehensive Cancer Network (NCCN) Guidelines.

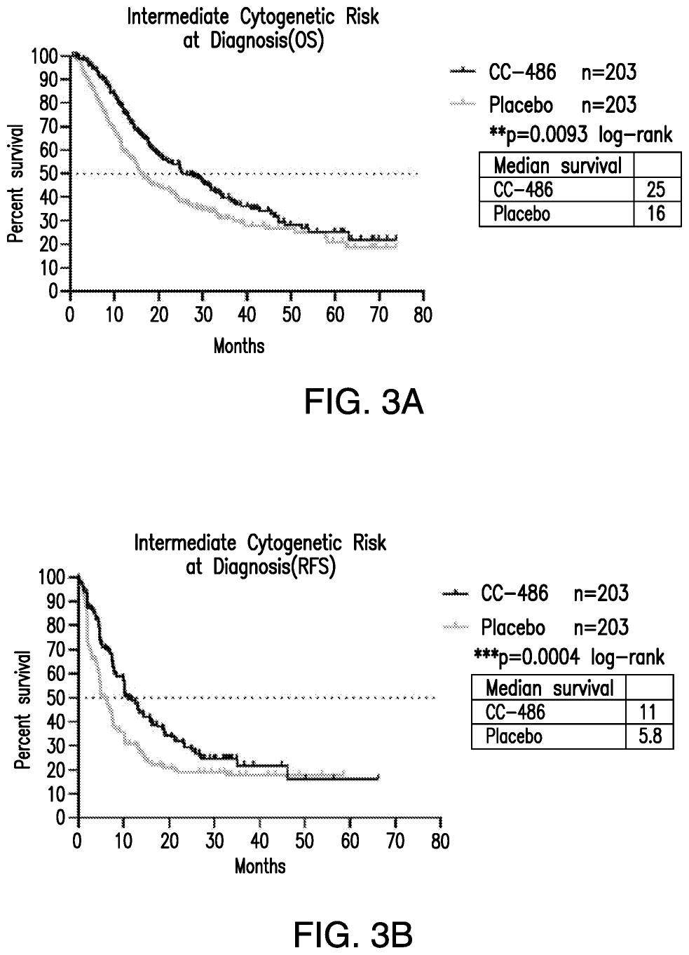

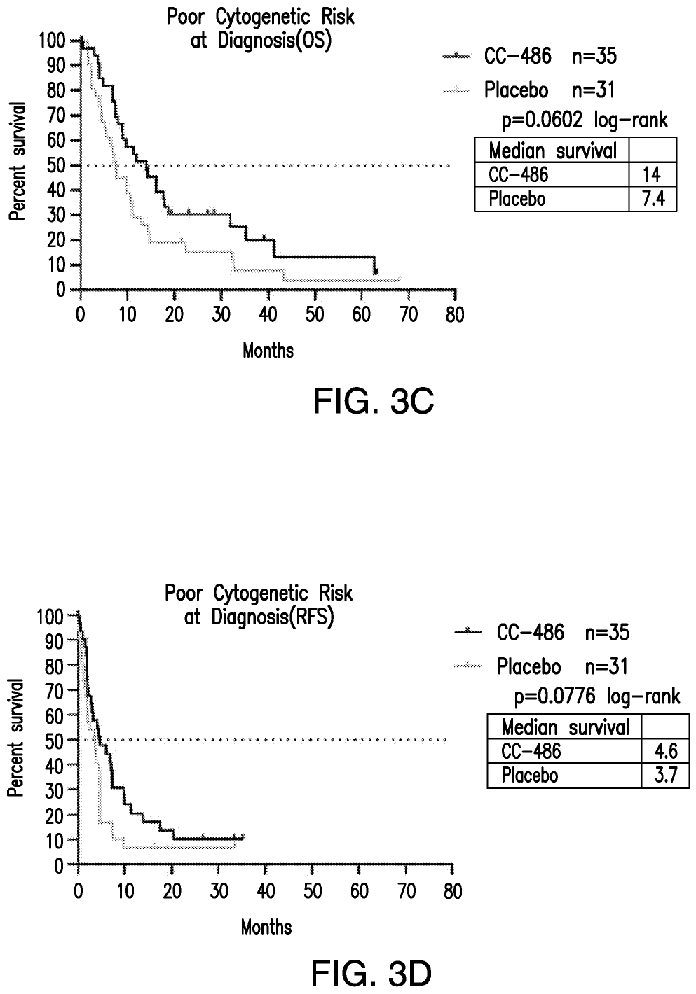

FIG. 3A to FIG. 3D represent cytogenetic risk (intermediate- or poor-risk) at diagnosis and associations with clinical response. FIG. 3A shows OS for patients with intermediate cytogenetic risk treated with either CC-486 or placebo. FIG. 3B shows RFS for patients with intermediate cytogenetic risk treated with either CC-486 or placebo. FIG. 3C shows OS for patients with poor cytogenetic risk treated with either CC-486 or placebo. FIG. 3D shows RFS for patients with poor cytogenetic risk treated with either CC-486 or placebo. In intermediate cytogenetic risk patients, statistically significant increases in OS and RFS were observed in patients treated with CC-486 versus placebo (median OS 25 months versus 16 months, respectively; p=0.0093; median RFS 11 months versus 5.8 months, p=0.0004). In poor cytogenetic risk patients, a trend for increases in OS and RFS were observed in patients treated with CC-486 versus placebo (median OS 14 months versus 7.4 months, respectively; p=0.0602; median RFS 4.6 months versus 3.7 months, respectively; p=0.0776). The Figures were generated using Kaplan-Meier analyses and p-values were calculated using log-rank test.

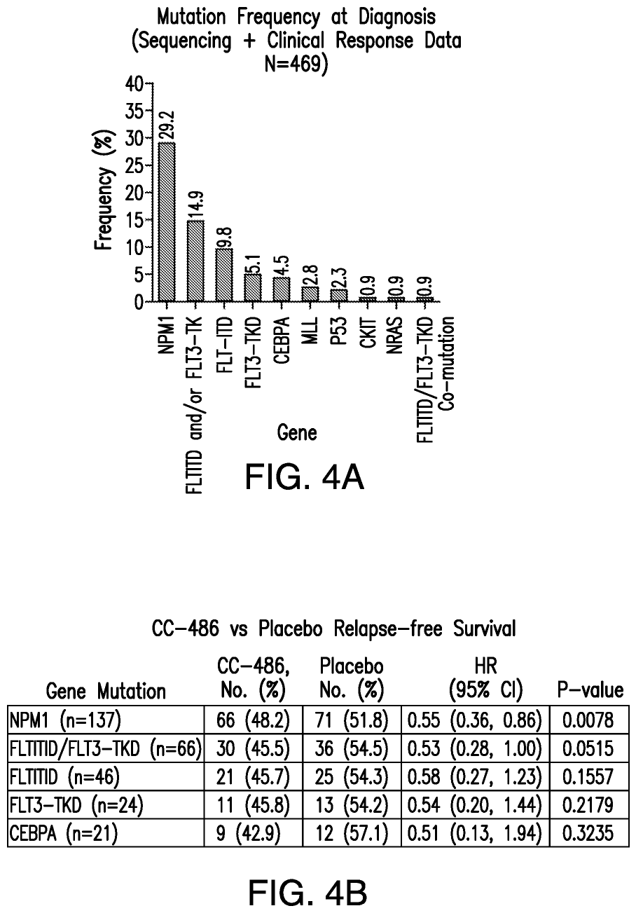

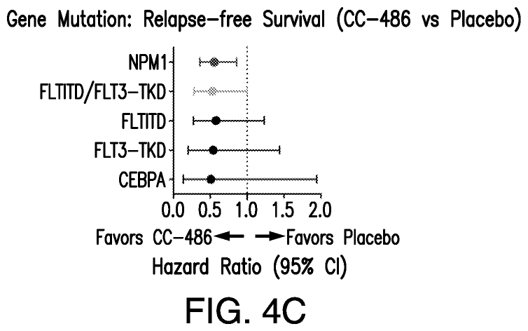

FIG. 4A to FIG. 4C represent prevalence of gene mutations at diagnosis and associated hazard ratios for RFS comparing CC-486 versus placebo. In FIG. 4A, the most frequently mutated gene was NPM1 (29.2%), followed by FLTITD and/or FLT3-TKD (14.9%), FLTITD (9.8%), FLT3-TKD (5.1%), CEBPA (4.5%), MLL (2.8%), p53 (2.3%), CKIT (0.9%), NRAS (0.9%), and FLTITD/FLT3-TKD co-mutation (0.9%). In FIG. 4B, hazard ratios (95% confidence intervals [CI]) and associated p-values are reported in the table; the data were visualized using a forest plot in FIG. 4C. A hazard ratio below 1.0 favors CC-486 treatment, and a hazard ratio above 1.0 favors placebo. NPM1 mutation had a hazard ratio of 0.55 (95% CI 0.36 to 0.86), which favored CC-486 treatment, and that was a statistically significant result (p=0.0078). Genes that were mutated in at least 20 patients were also analyzed; FLTITD and/or FLT3-TKD, FLTITD, FLT3-TKD, and CEBPA had hazard ratios below 1.0; however, the associated p-values were not statistically significant. Cox regression analyses were performed to evaluate the association of gene mutation status and treatment arm with RFS.

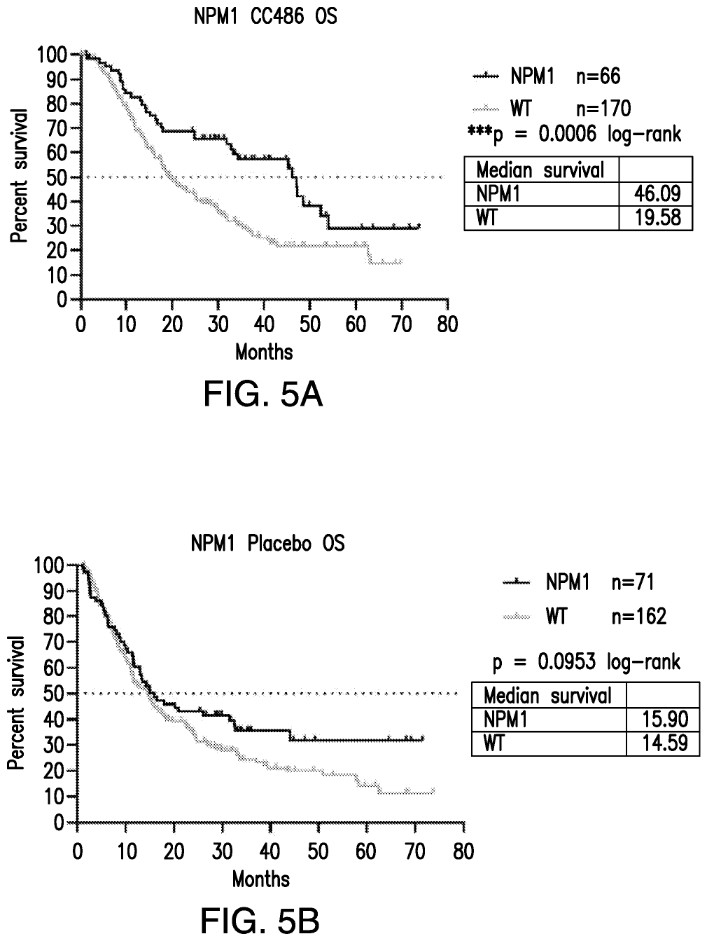

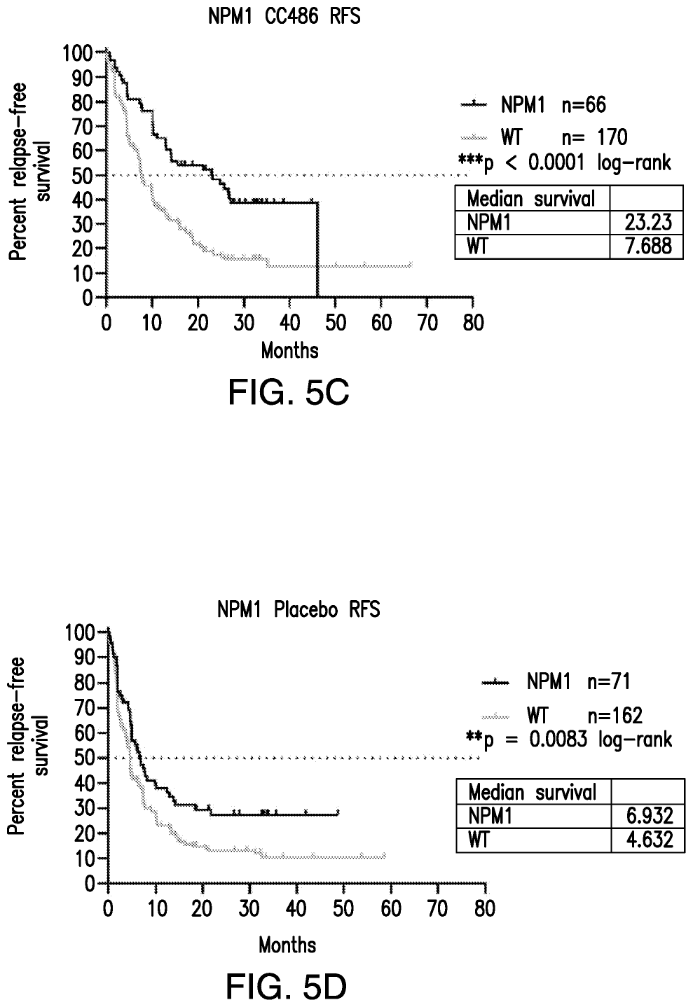

FIG. 5A to FIG. 5D represent NPM1 mutation status (NPM1 mutation versus NPM1 wild type) at diagnosis and associations with clinical response. FIG. 5A shows OS for patients with NPM1 mutation versus NPM1 wild type treated with CC-486. FIG. 5B shows OS for patients with NPM1 mutations versus NPM1 wild type treated with placebo. FIG. 5C shows RFS for patients with NPM1 mutations versus NPM1 wild type treated with CC-486. FIG. 5D shows RFS for patients with NPM1 mutation versus NPM1 wild type treated with placebo. In FIG. 5A, a statistically significant increase in OS was observed in patients harboring NPM1 mutation versus patients with NPM1 wild type and treated with CC-486 (median OS 46.09 months versus 19.58 months, respectively; p=0.0006). In FIG. 5B, a trend for prolonged OS was observed in patients harboring NPM1 mutation versus NPM1 wild type treated with placebo (median OS 15.90 months versus 14.59 months, respectively; p=0.0953). In FIG. 5C and FIG. 5D, statistically significant increases in OS and RFS were observed in patients harboring NPM1 mutation versus patients with NPM1 wild type and treated with CC-486 (median RFS 23.23 months versus 7.688 months, respectively; p<0.0001) or placebo (median RFS 6.932 months versus 4.632 months, respectively; p=0.0083). Figures were generated using Kaplan-Meier analyses and p-values were calculated using log-rank test.

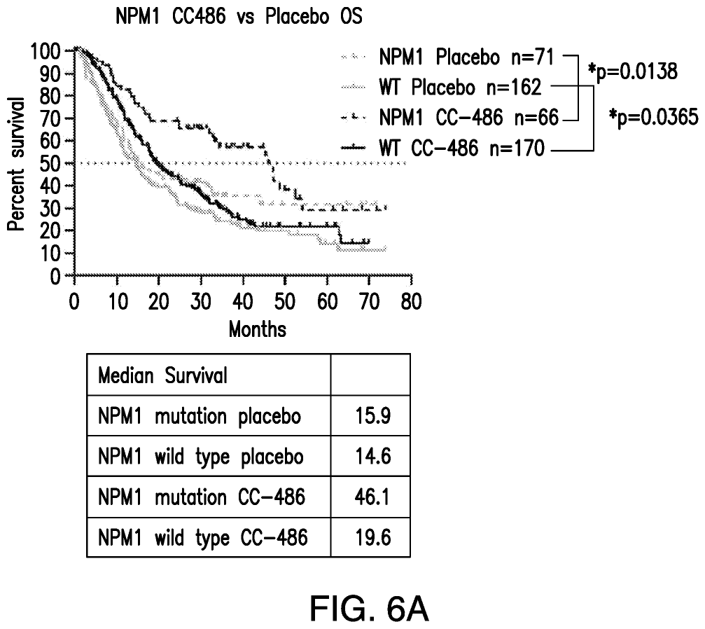

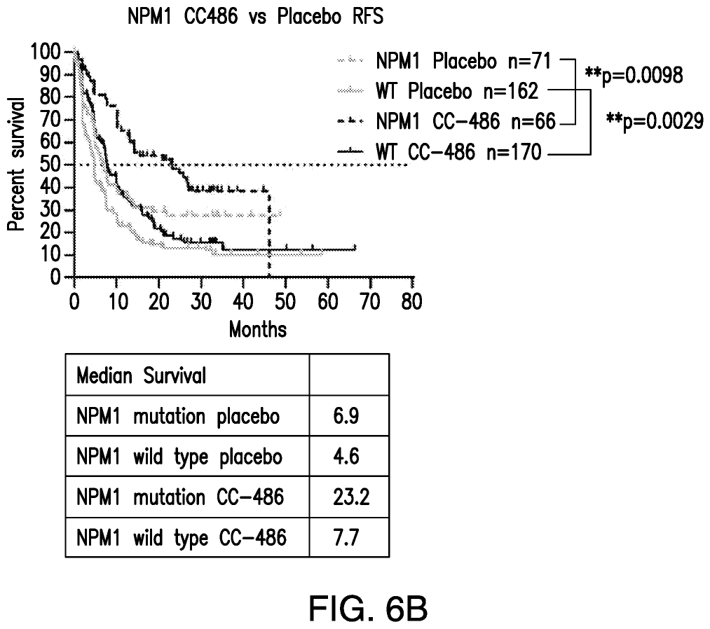

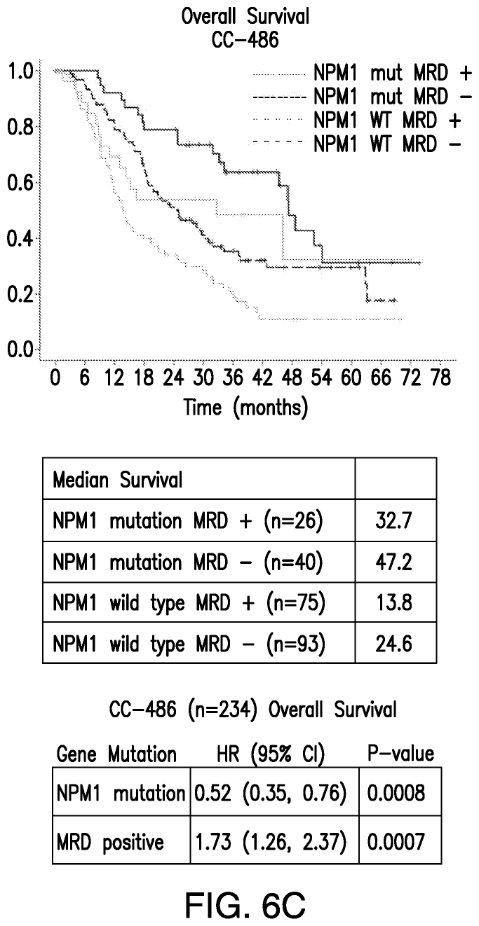

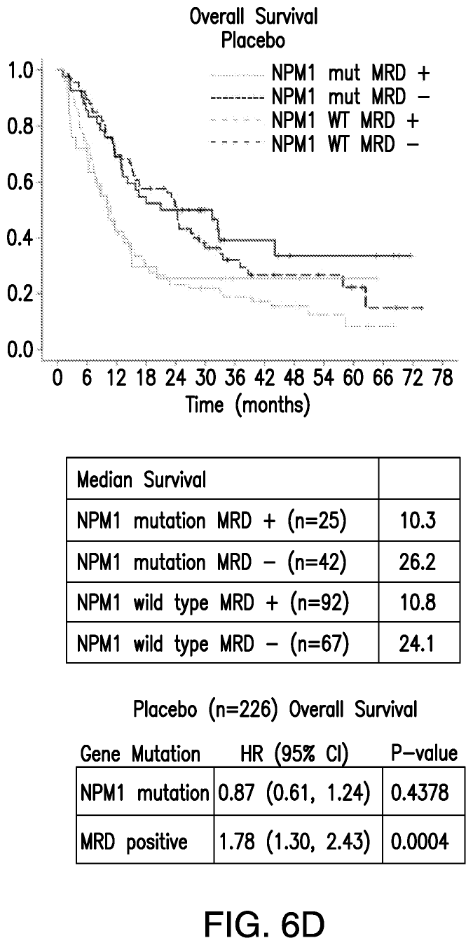

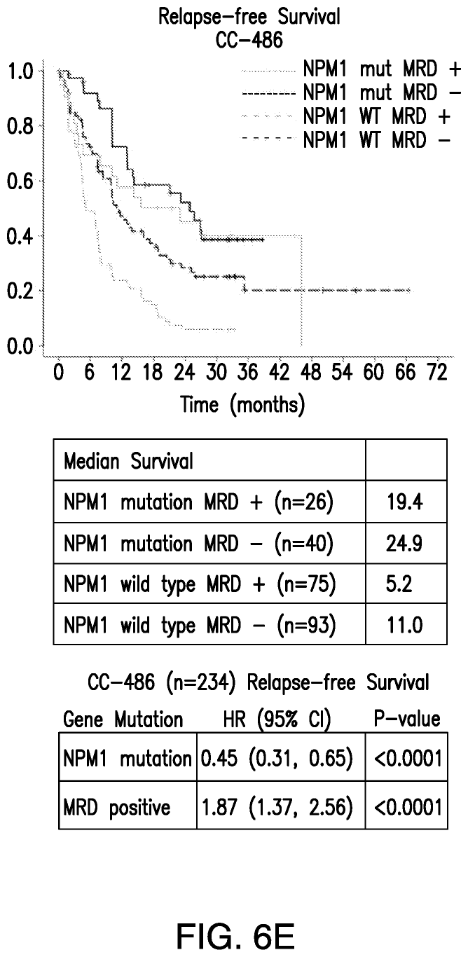

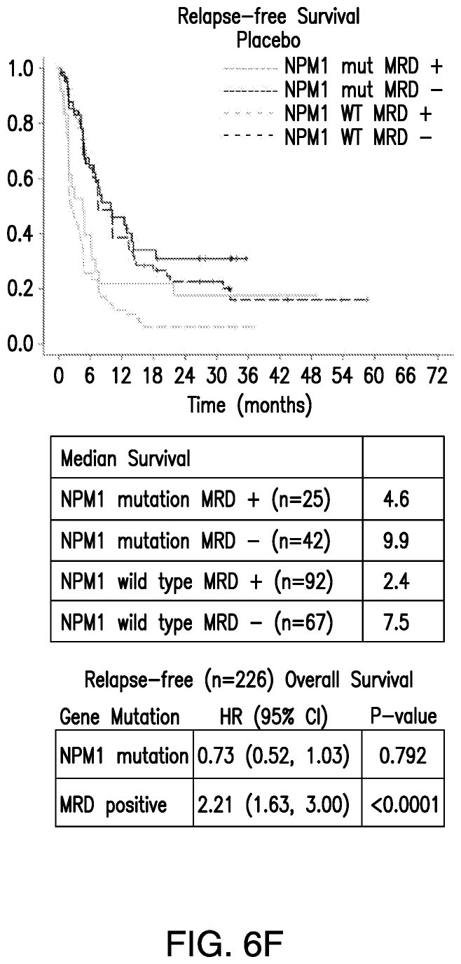

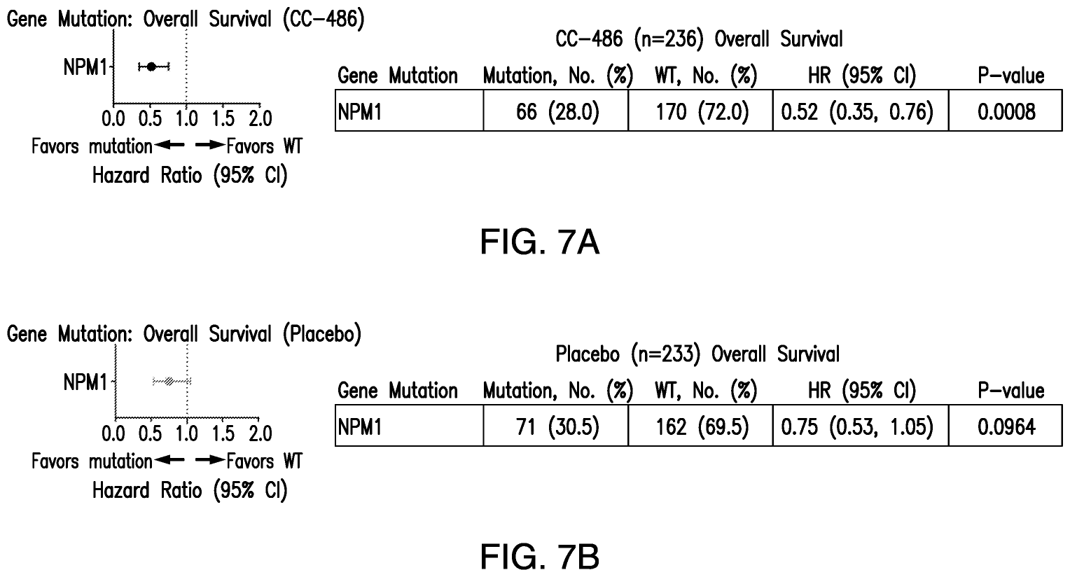

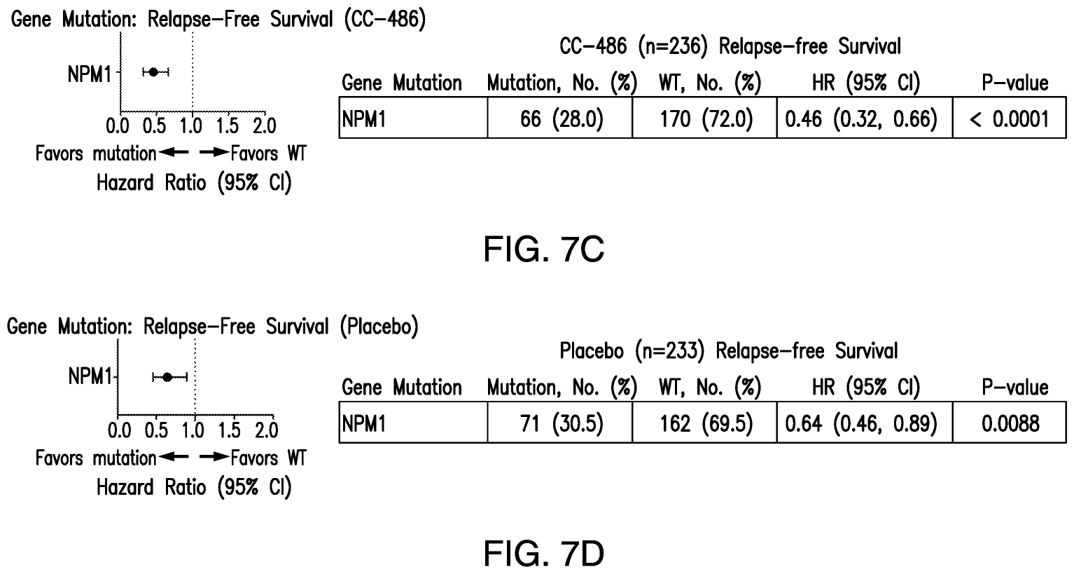

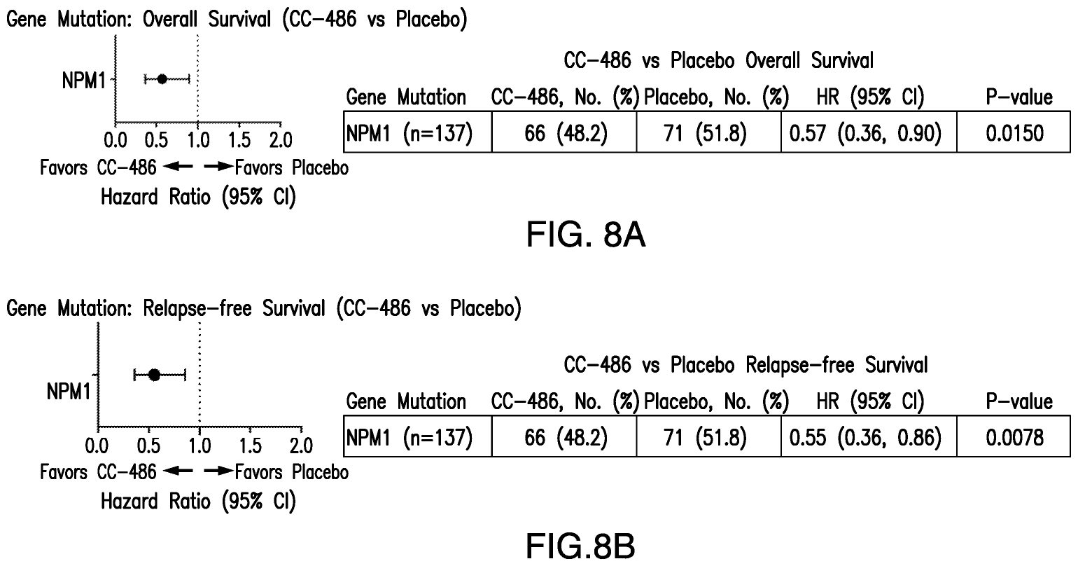

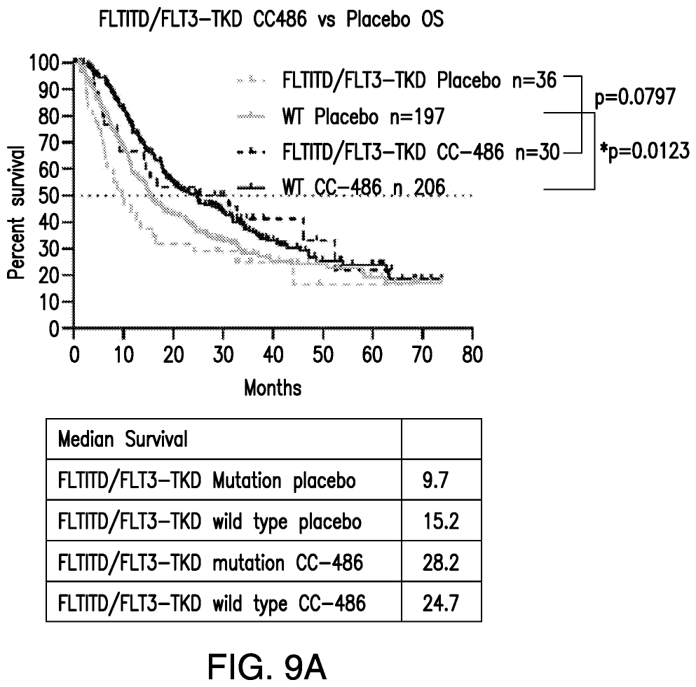

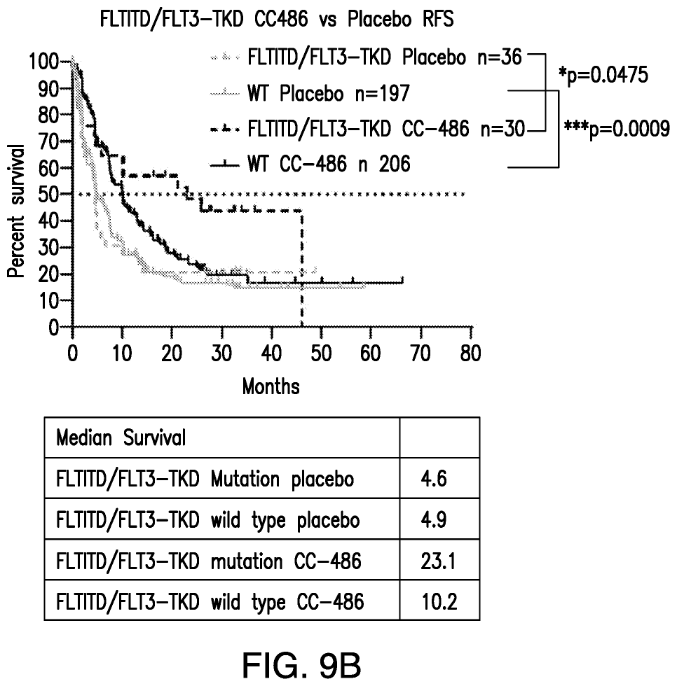

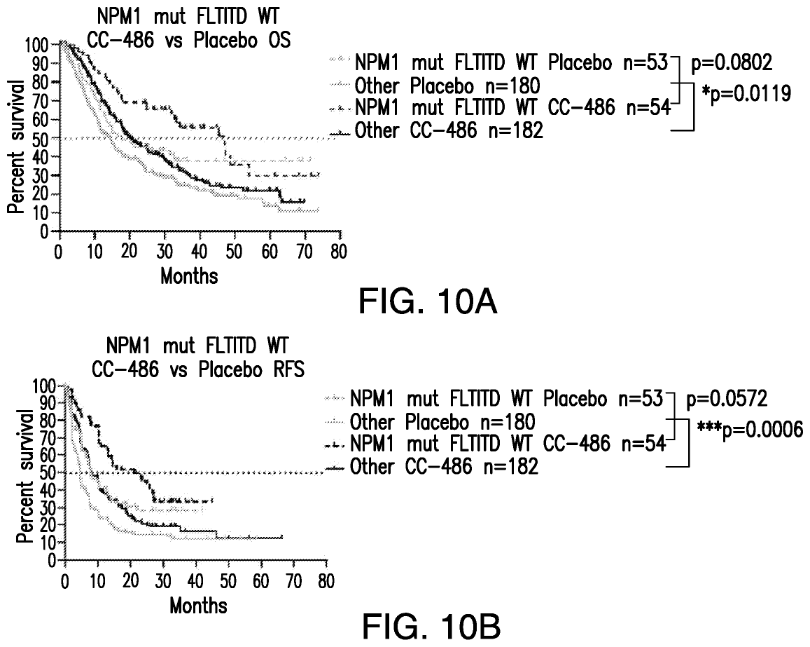

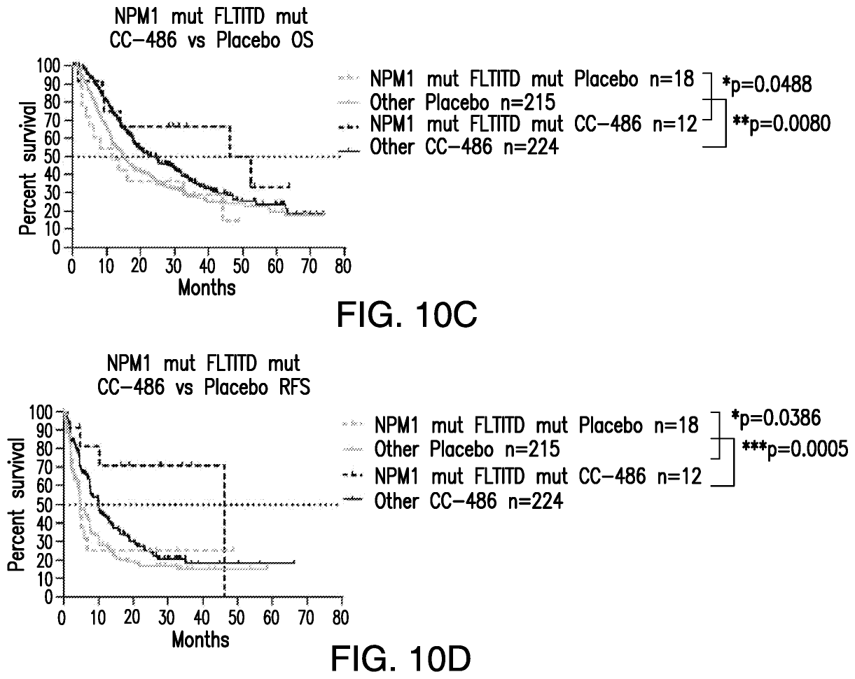

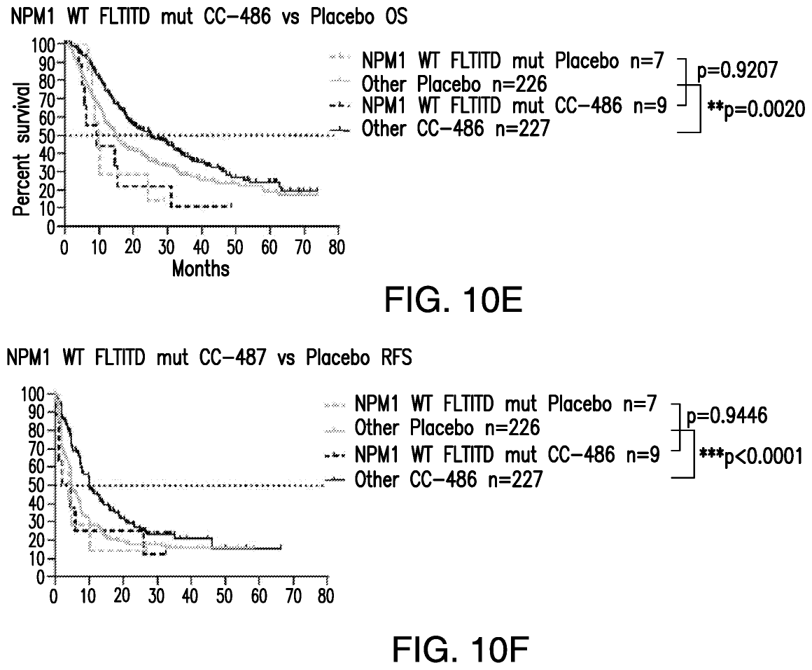

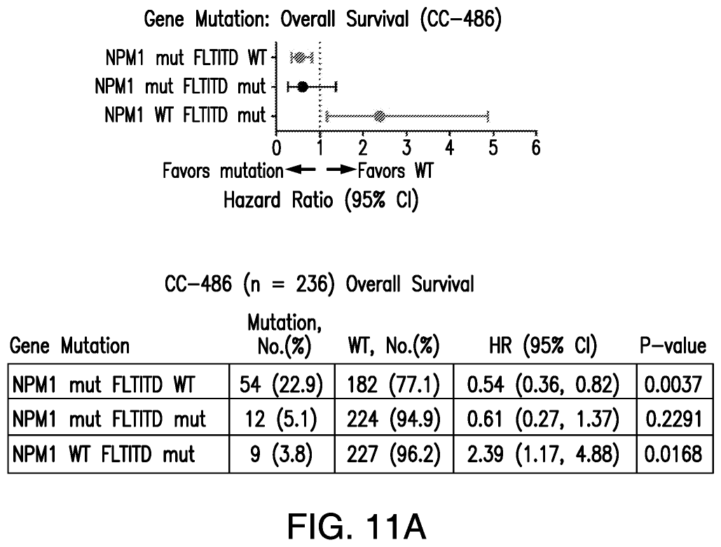

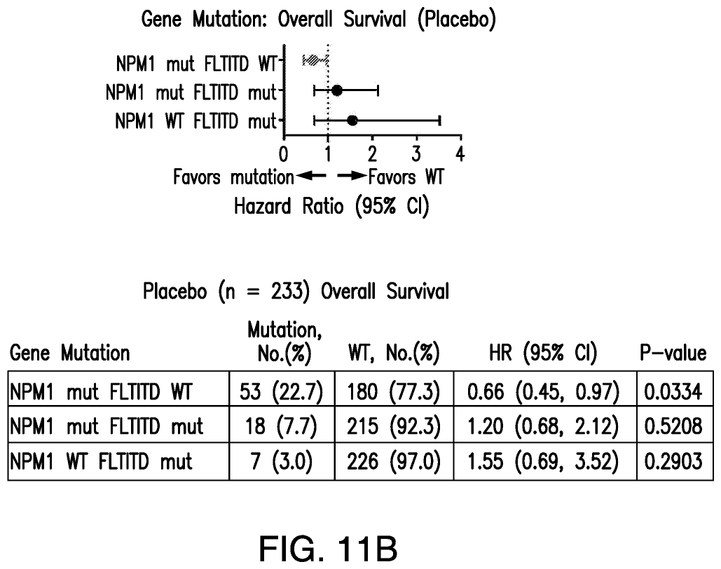

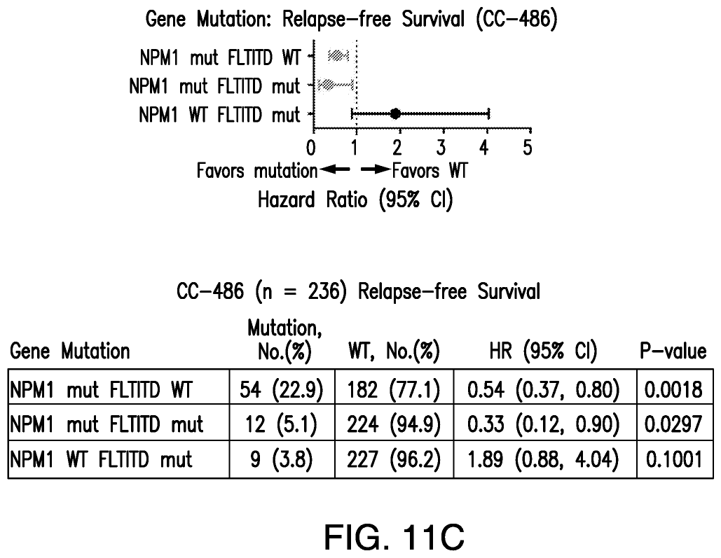

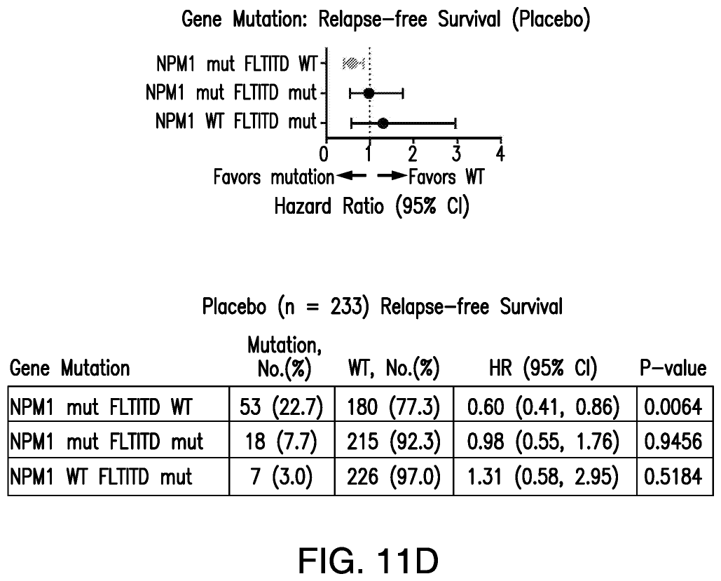

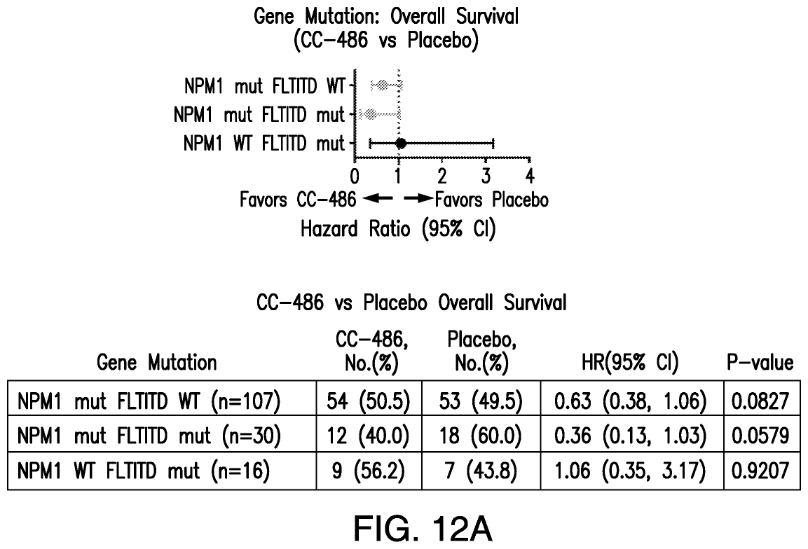

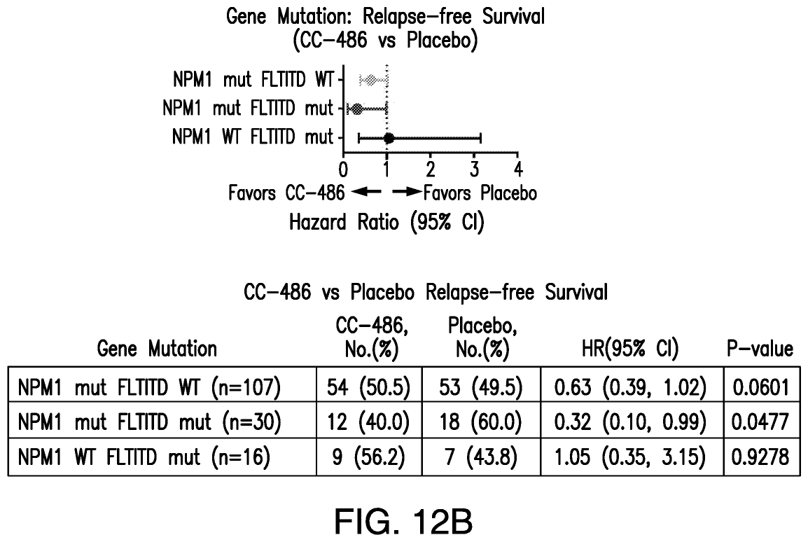

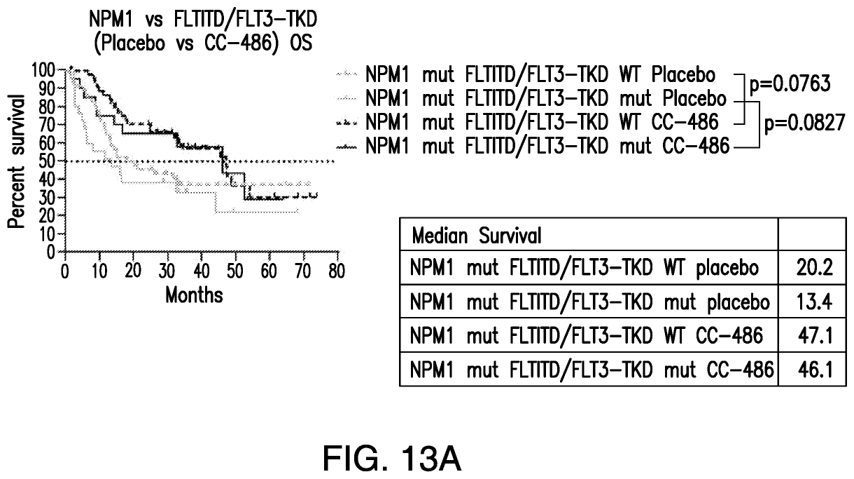

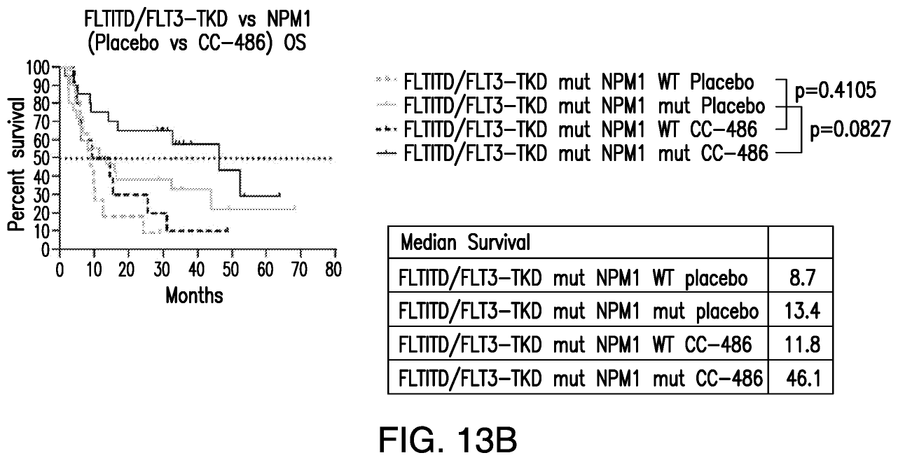

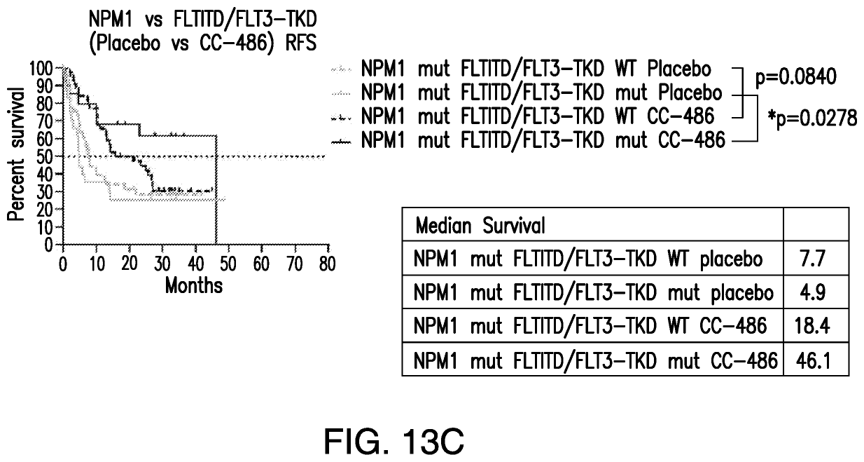

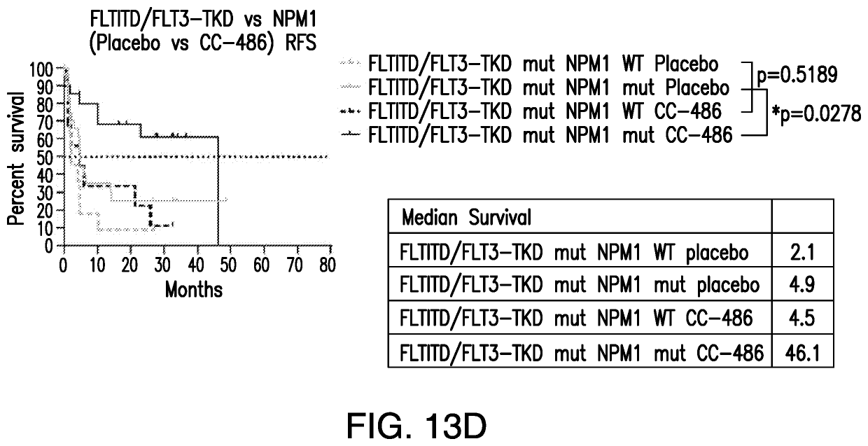

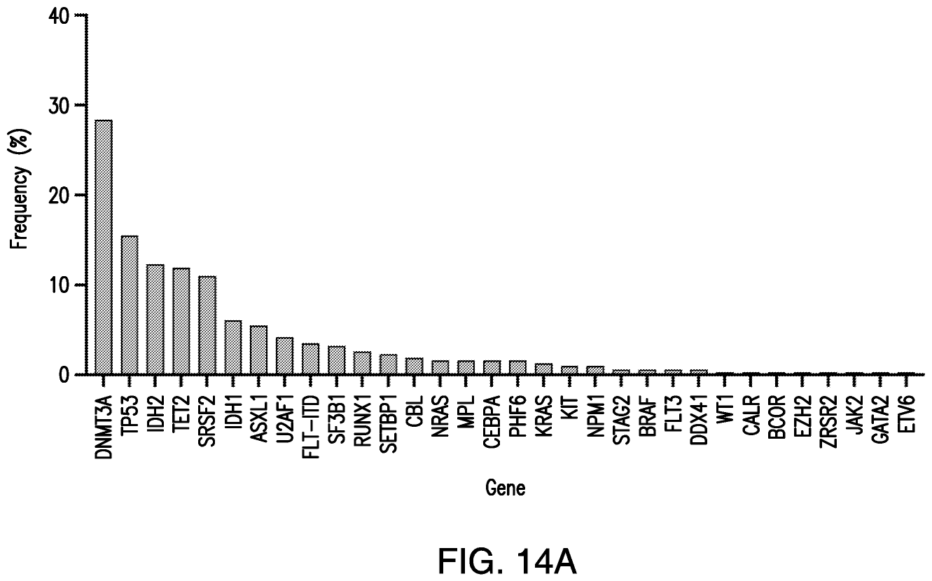

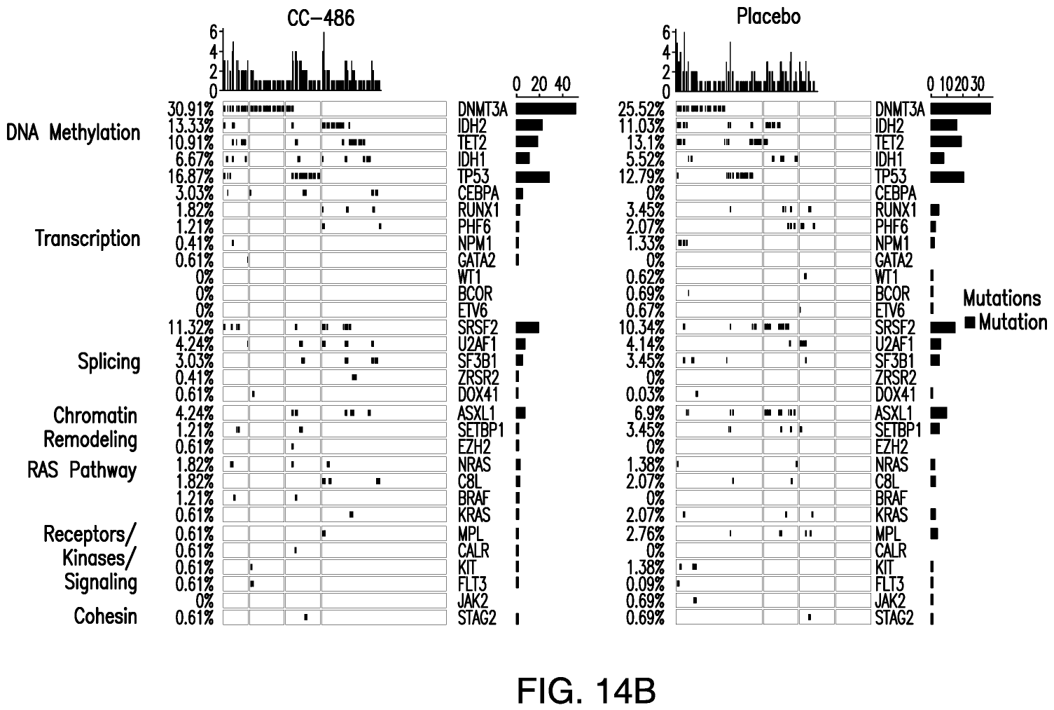

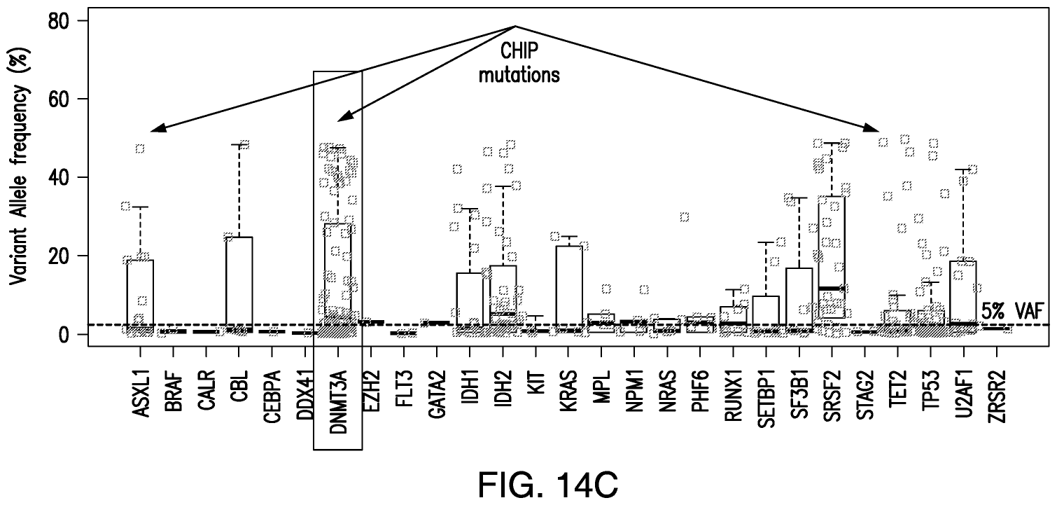

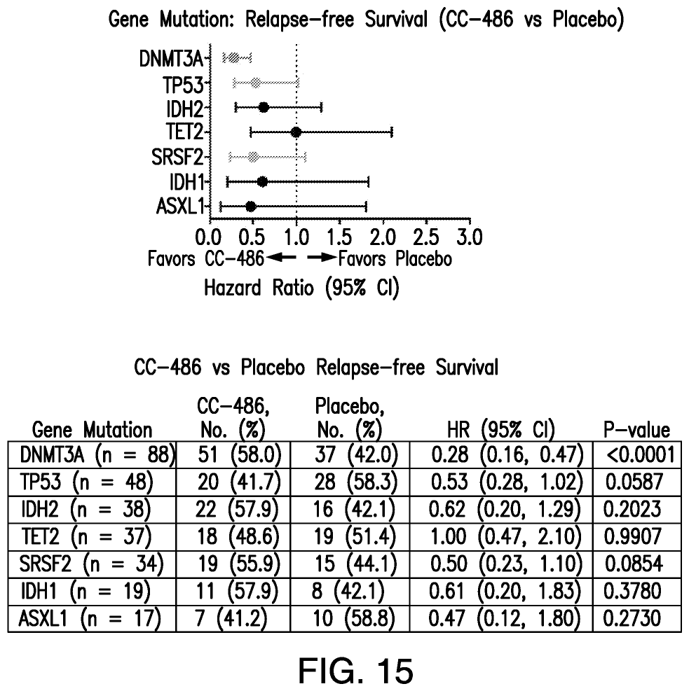

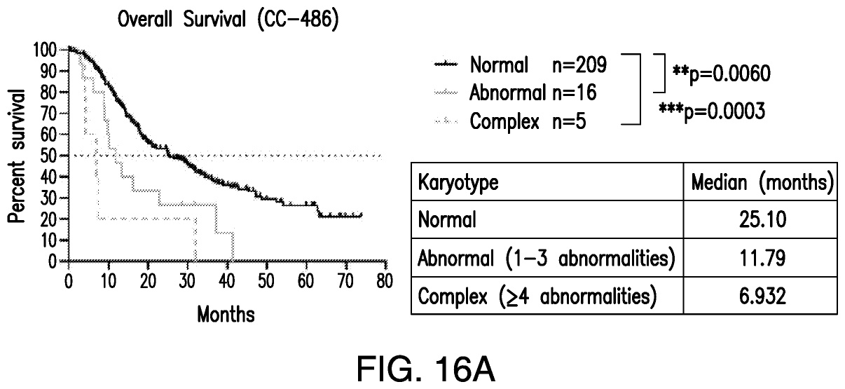

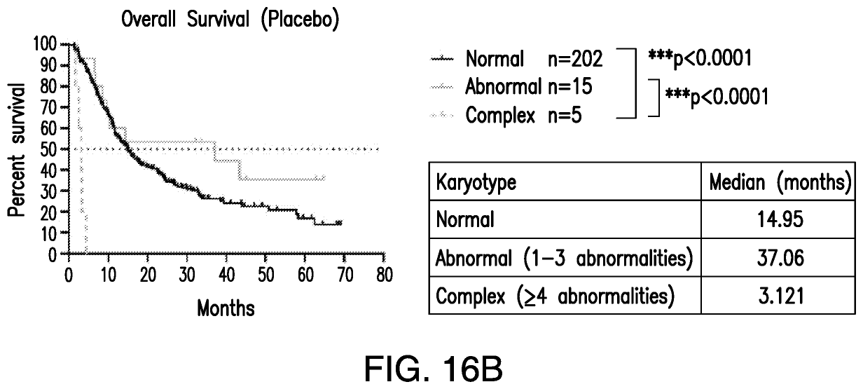

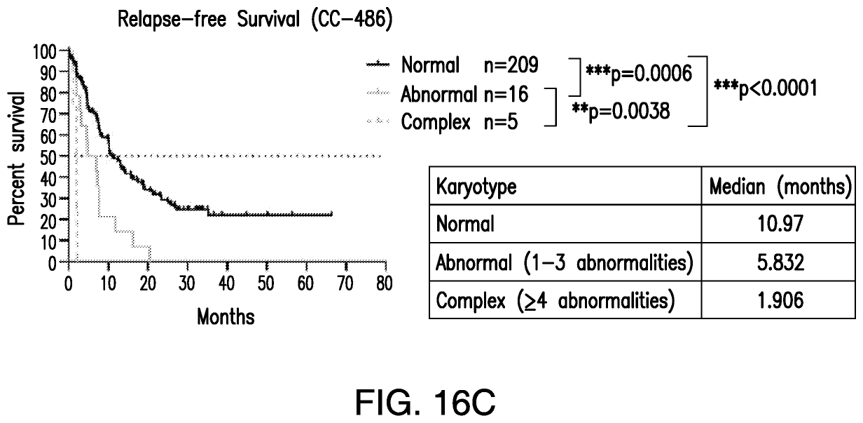

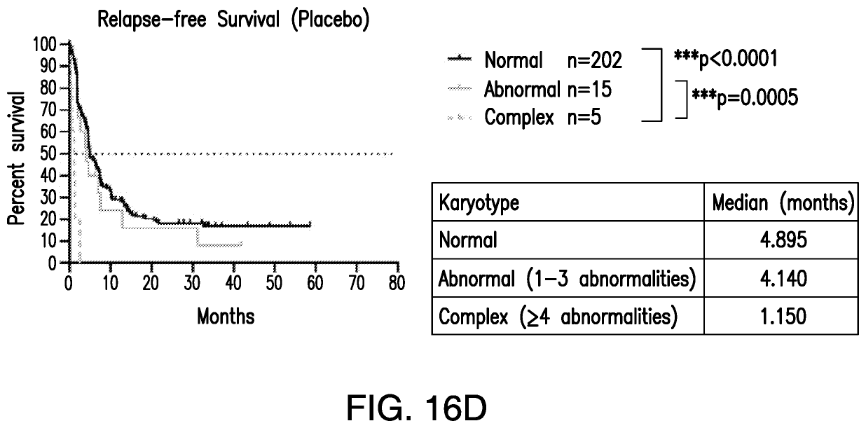

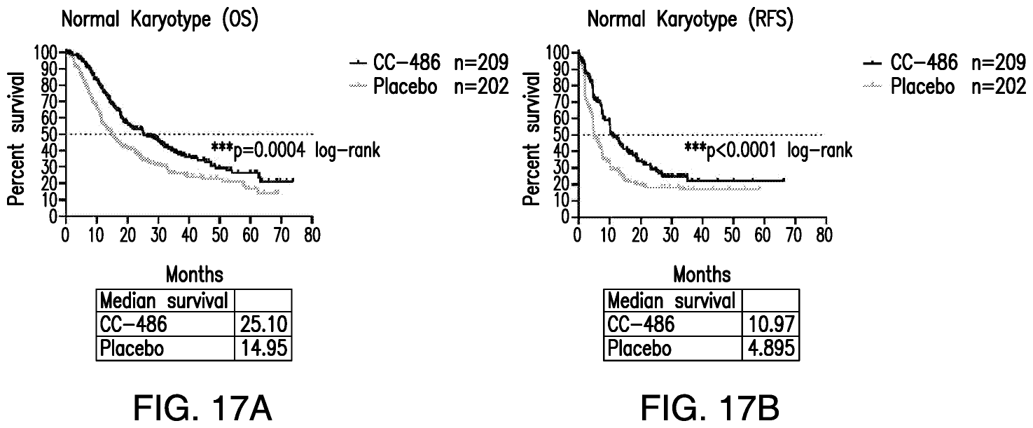

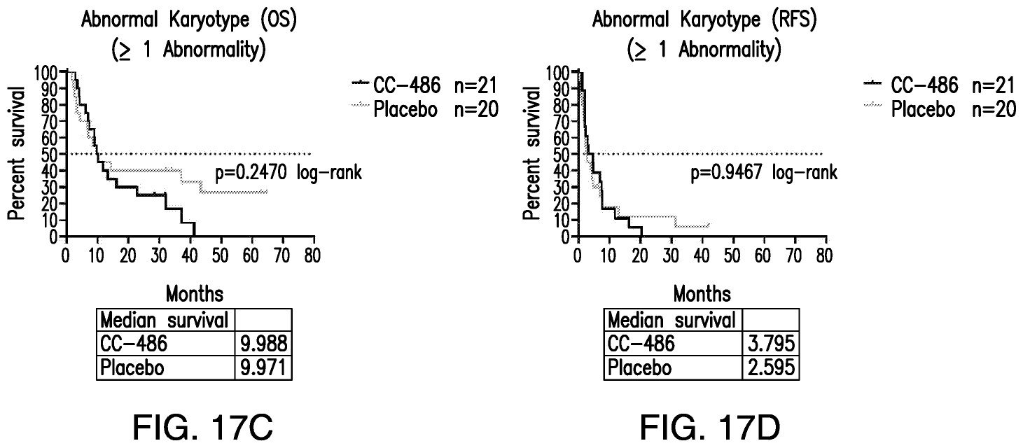

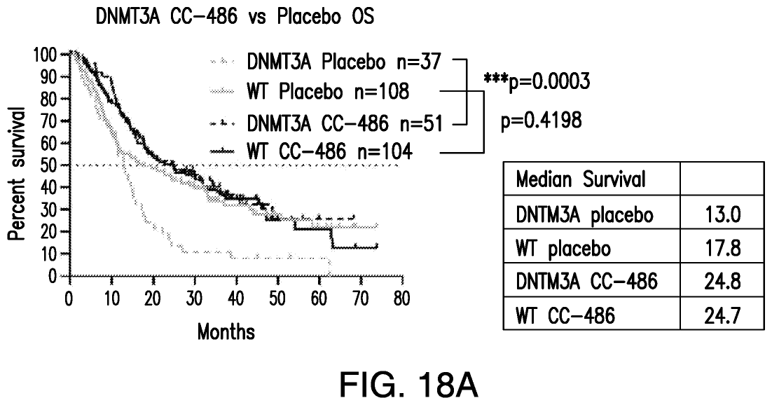

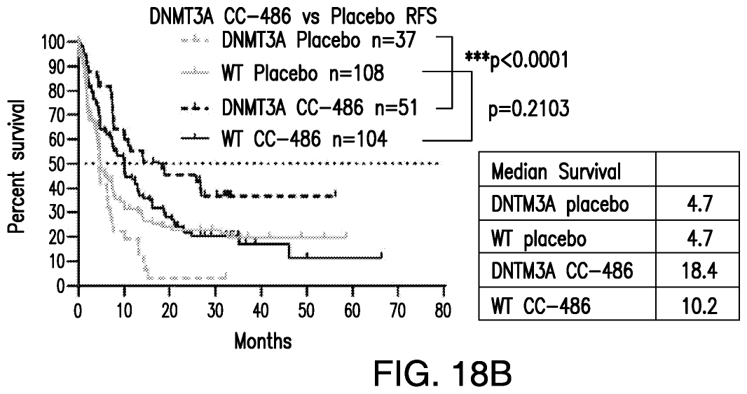

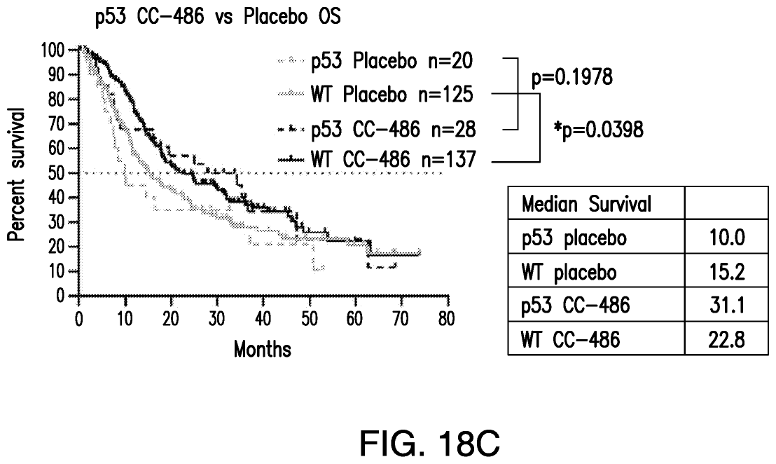

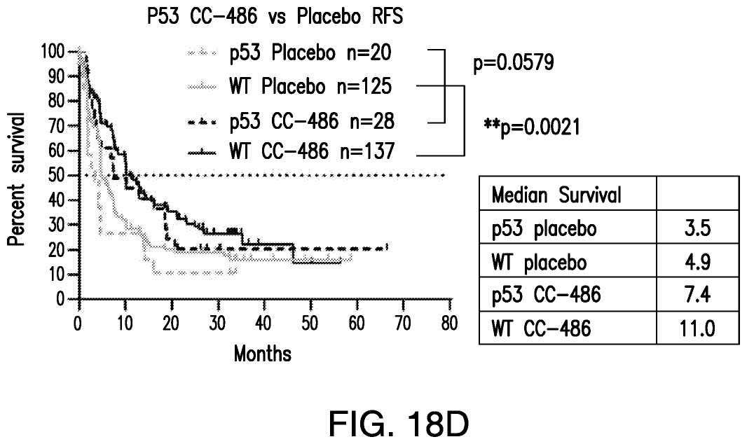

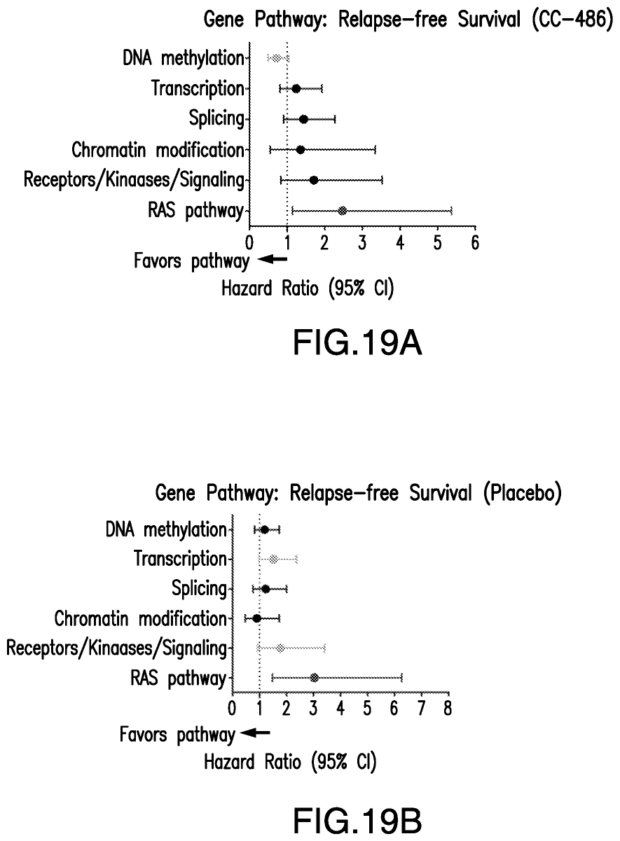

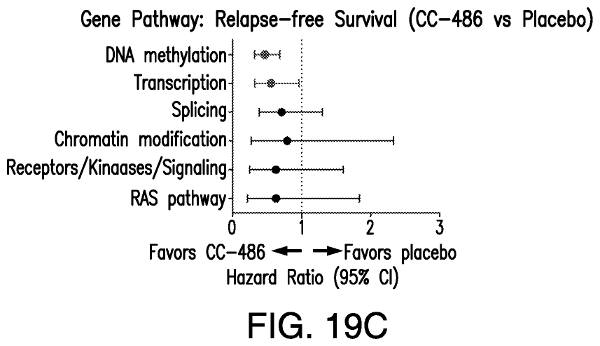

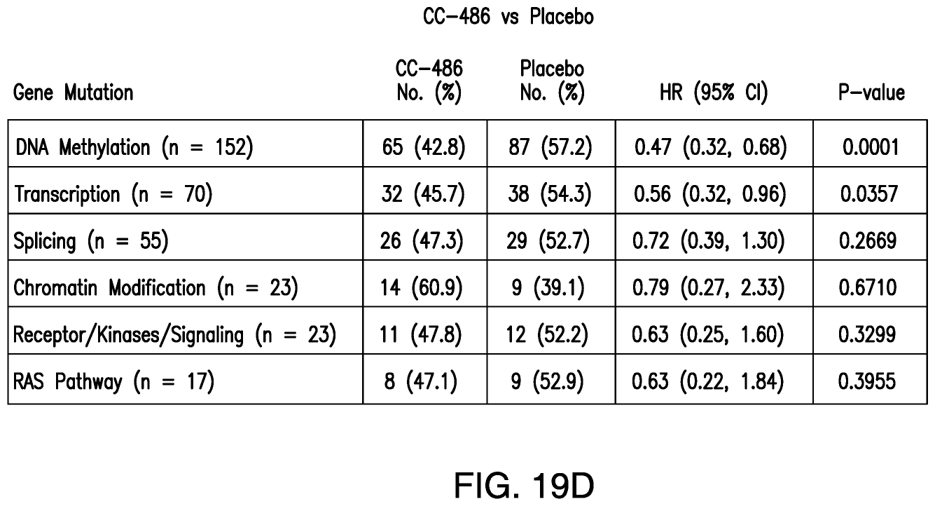

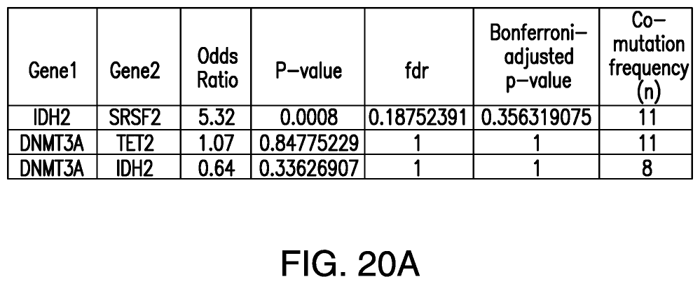

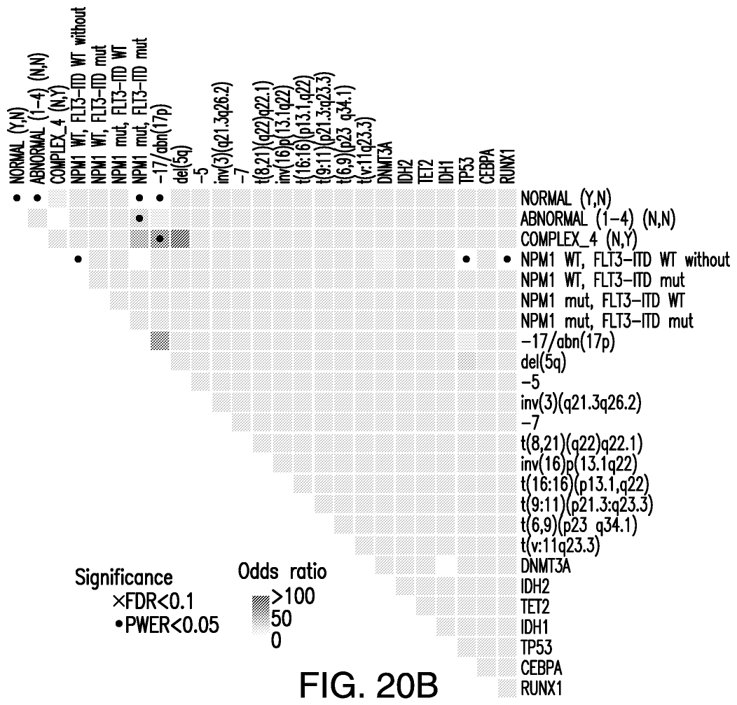

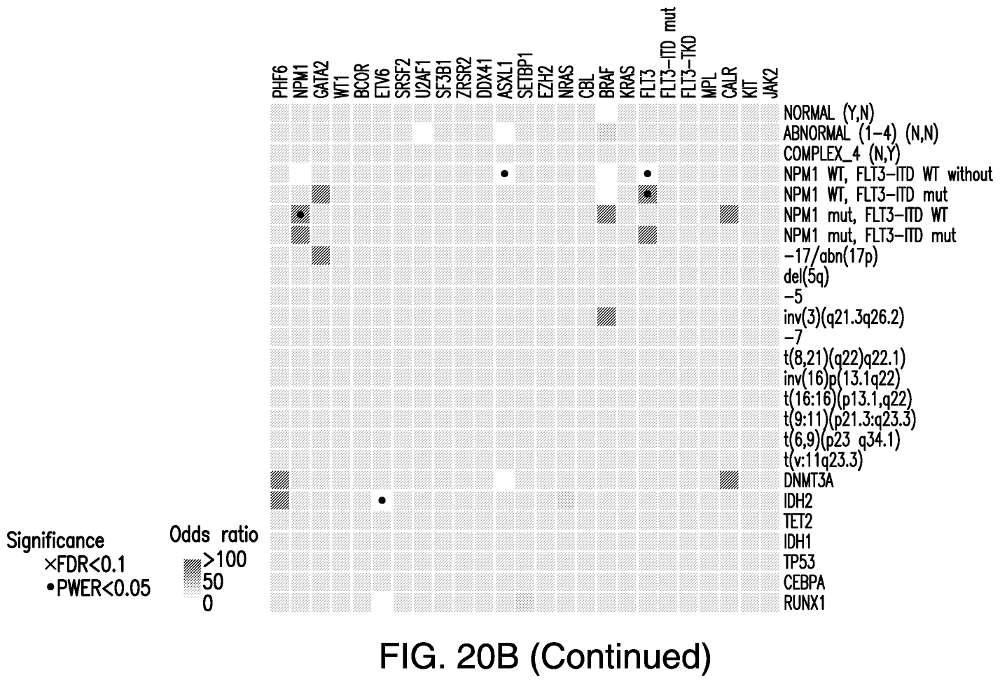

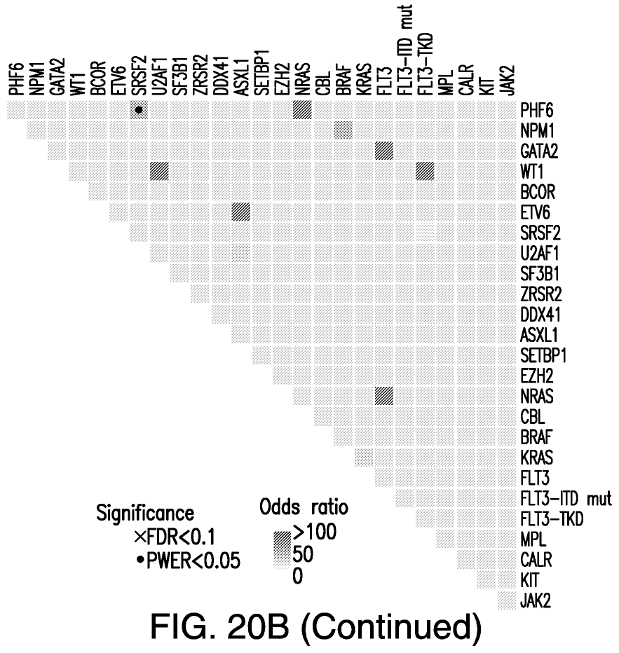

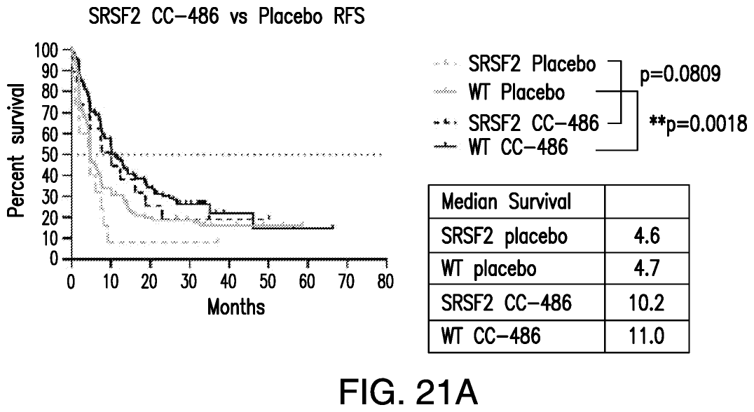

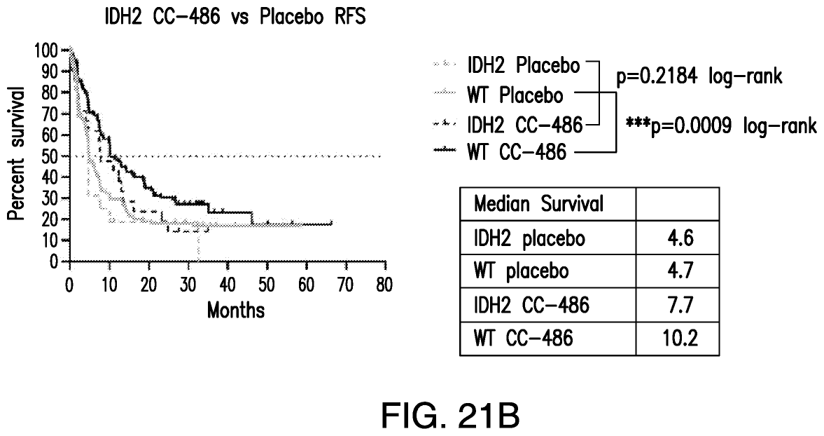

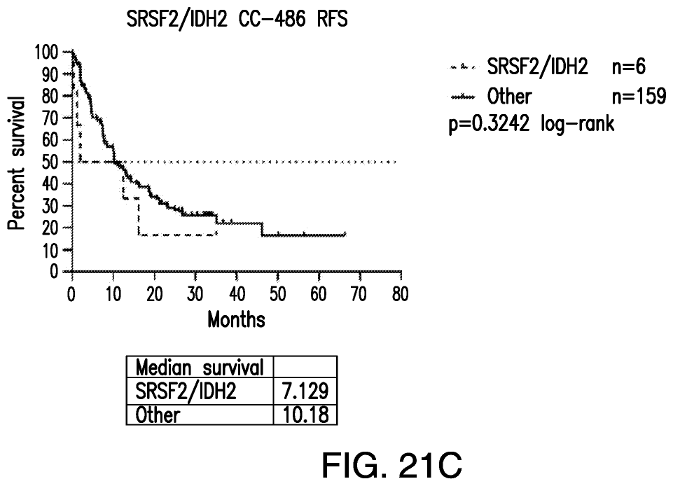

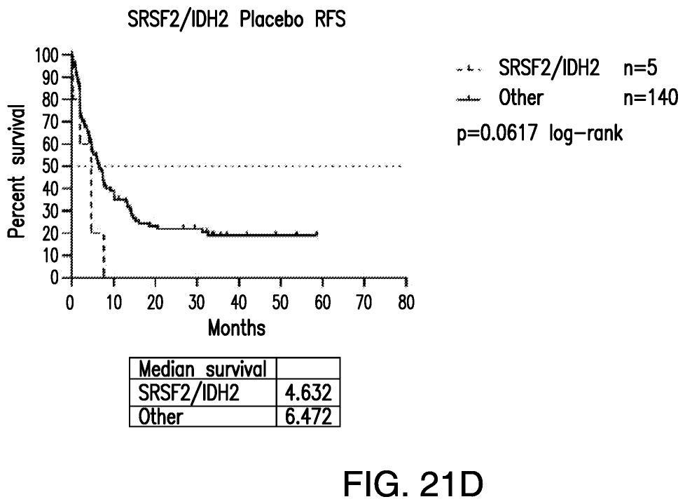

FIG. 6A and FIG. 6B represent NPM1 mutation status at diagnosis and associations with clinical response in patients treated with CC-486 versus placebo. FIG. 6C to FIG. 6F represent multivariate analysis of NPM1 mutation status at diagnosis and minimal residual disease (MRD) at screening and associations with clinical response. FIG. 6A shows overall survival (OS) for patients harboring NPM1 mutation or NPM1 wild type and treated with either placebo or CC-486. FIG. 6B shows relapse-free survival (RFS) for patients harboring NPM1 mutation or NPM1 wild type and treated with either placebo or CC-486. FIGS. 6C and 6D shows overall survival (OS) analysis and hazard ratios comparing NPM1 mutation status in combination with MIRD status in patients treated with CC-486 or placebo. FIGS. 6E and 6F shows relapse-free survival (RFS) analysis and hazard ratios comparing NPM1 mutation status in combination with MIRD status in patients treated with CC-486 or placebo. In FIG. 6A, a significantly prolonged increase in OS was observed comparing patients harboring NPM1 mutation and treated with CC-486 versus placebo (median OS 46.1 months versus 15.9 months, respectively; p=0.0138). A significant increase in OS was observed in NPM1 wild type patients treated with CC-486 versus placebo (median OS 19.6 months versus 14.6 months, respectively; p=0.0365). In FIG. 6B, a significantly prolonged increase in RFS was observed in patients harboring NPM1 mutation and treated with CC-486 versus placebo (median OS 23.2 months versus 6.9 months, respectively; p=0.0098). A significant increase in RFS was observed in NPM1 wild type patients treated with CC-486 versus placebo (median RFS 7.7 months versus 4.6 months, respectively; p=0.0029). A larger fraction of patients with NPM1 mutation were MRD negative (61.7%) than were MRD positive (38.4%) at screen compared to patients with wild type NPM1 (p=0.0178 by Fisher's exact test) (FIG. 6C to FIG. 6F). In FIG. 6C, prolonged increases in OS was observed in CC-486-treated patients with NPM1 mutation and MRD negative status (median OS 47.2 months) or NPM1 mutation and MRD positive status (median OS 32.7 months) compared to patients with NPM1 wild type and MRD negative (median OS 24.6 months) or NPM1 wild type and MRD positive status (median OS 13.8 months). A hazard ratio below 1.0 favors NPM1 mutation or MRD positive status, and a hazard ratio above 1.0 favors wild type NPM1 or MRD negative status. In FIG. 6C, the hazard ratio for NPM1 status was 0.52 (p=0.0008), showing that NPM1 mutation was associated with better survival in CC-486-treated patients and the hazard ratio for MRD status was 1.73 (p=0.0007), showing that MRD positive status was associated with poor survival. These results also showed that NPM1 mutation status was associated with improved survival beyond the effects of MRD status. In FIG. 6D, NPM1 mutation status was not improved in placebo-treated patients compared to wild type NPM1 (median OS NPM1 mutation and MRD negative 26.2 months; median OS NPM1 mutations and MIRD positive 10.3 months); only MRD negative status was associated with improved survival independent of NPM1 status in placebo-treated patients (median OS wild type NPM1 and MIRD negative 24.1 months; medina OS wild type NPM1 and MRD positive 10.8 months). Furthermore, the hazard ratio for NPM1 mutation in placebo-treated patients was 0.87 (p=0.4378) and MRD positive status was 1.78 (0.0004), which showed that NPM1 mutation was not associated with improved survival, whereas MRD positive status was associated with poor survival in placebo-treated patients. Similar results were observed for relapse-free survival outcomes (FIG. 6E and FIG. 6F). Figures were generated using Kaplan-Meier analyses, and multivariate Cox regression analyses were performed to compare the associations of NPM1 mutation status and MRD status with clinical outcomes (OS or RFS), separated by treatment arm.

FIG. 7A to FIG. 7D represent NPM1 mutation status at diagnosis and associated hazard ratios for OS and RFS in patients treated with CC-486 or placebo. In FIG. 7A, OS hazard ratio for NPM1 mutation versus NPM1 wild type in CC-486 treated patients was visualized using a forest plot (left) and reported in the table (right) along with 95% confidence interval [CI] and associated p-value. In FIG. 7B, OS hazard ratio for NPM1 mutation versus NPM1 wild type in placebo treated patients was visualized using a forest plot (left) and reported in the table (right) along with 95% confidence interval (CI) and associated p-value. In FIG. 7C, RFS hazard ratio for NPM1 mutation versus NPM1 wild type in CC-486 treated patients was visualized using a forest plot (left) and reported in the table (right) along with 95% confidence interval (CI) and associated p-value. In FIG. 7D, RFS hazard ratio for NPM1 mutation versus NPM1 wild type in placebo treated patients was visualized using a forest plot (left) and reported in the table (right) along with 95% confidence interval (CI) and associated p-value. A hazard ratio below 1.0 favors NPM1 mutation and a hazard ratio above 1.0 favors NPM1 wild type. In FIG. 7A, OS hazard ratio for CC-486 was 0.52 (95% CI 0.35 to 0.76), which favored NPM1 mutation versus wild type, and this was a statistically significant result (p=0.0008). In FIG. 7B, OS hazard ratio for placebo was 0.75 (95% CI 0.53 to 1.05), which favored NPM1 mutation versus wild type, with a trend for significance (p=0.0964). In FIG. 7C, RFS hazard ratio for CC-486 was 0.46 (95% CI 0.32 to 0.66), which favored NPM1 mutation versus wild type, and this was a statistically significant result (p<0.0001). In FIG. 7D, RFS hazard ratio for placebo was 0.64 (95% CI 0.46 to 0.89), which favored NPM1 mutation versus wild type, and this was a statistically significant result (p=0.0088). Cox regression analyses were performed to evaluate the association of gene mutation status and treatment arm with OS and RFS.

FIG. 8A and FIG. 8B represent hazard ratios for patients harboring NPM1 mutations and associations with clinical response in patients treated with CC-486 versus placebo. In FIG. 8A, OS hazard for patients with NPM1 mutations in CC-486 versus placebo was visualized using a forest plot (left) and reported in the table (right) along with 95% CI and associated p-value. In FIG. 8B, RFS hazard ratio for patients with NPM1 mutations in CC-486 versus placebo was visualized using a forest plot (left) and reported in the table (right) along with 95% CI and associated p-value. A hazard ratio below 1.0 favors CC-486 and a hazard ratio above 1.0 favors placebo. The OS hazard ratio for NPM1 mutated AML was 0.57 (95% CI 0.36 to 0.90), which favored CC-486 treatment versus placebo, and this was a statistically significant result (p=0.0150). The RFS hazard ratio for NPM1 mutated AML was 0.55 (95% CI 0.36 to 0.86), which favored CC-486 treatment versus placebo, and this was a statistically significant result (p=0.0078). Cox regression analyses were performed to evaluate the association of gene mutation status and treatment arm with OS and RFS.

FIG. 9A and FIG. 9B represent FLT3 mutation status (FLTITD and/or FLT3-TKD) at diagnosis and associations with clinical response in patients treated with CC-486 versus placebo. FIG. 9A shows overall survival (OS) for patients harboring FLTITD/FLT3-TKD mutation or FLTITD/FLT3-TKD wild type and treated with either placebo or CC-486. FIG. 9B shows RFS for patients harboring FLTITD/FLT3-TKD mutation or FLTITD/FLT3-TKD wild type and treated with either placebo or CC-486. In FIG. 9A, a trend for prolonged OS was observed comparing patients harboring FLTITD/FLT3-TKD mutation and treated with CC-486 versus placebo (median OS 28.2 months versus 9.7 months, p=0.0797). A statistically significant increase in OS was observed in FLTITD/FLT3-TKD wild type patients treated with CC-486 versus placebo (median OS 24.7 months versus 15.2 months, respectively; p=0.0123). In FIG. 9B, a significantly prolonged increase in RFS was observed for patients harboring FLTITD/FLT3-TKD mutation and treated with CC-486 versus placebo (median OS 23.1 months versus 4.6 months, respectively; p=0.0475). A significant increase in RFS was observed in FLTITD/FLT3-TKD wild type patients treated with CC-486 versus placebo (median RFS 10.2 months versus 4.9 months, respectively; p=0.0009). Figures were generated using Kaplan-Meier analyses and p-values were calculated using log-rank test.

FIG. 10A to FIG. 10F represent NPM1 in combination with FLTITD mutation status at diagnosis and associations with clinical response in patients treated with CC-486 versus placebo. FIG. 10A and FIG. 10B show OS and RFS for patients harboring NPM1 mutation and FLTITD wild type and treated with either placebo or CC-486. FIG. 10C and FIG. 10D show OS and RFS for patients harboring NPM1 mutation and FLTITD mutation and treated with either placebo or CC-486. FIG. 10E and FIG. 10F show OS and RFS for patients harboring NPM1 wild type and FLTITD mutation and treated with either placebo or CC-486. In FIG. 10A, a trend for prolonged OS was observed comparing patients harboring NPM1 mutation FLTITD WT and treated with CC-486 versus placebo (median OS 47.1 months versus 18.0 months, p=0.0802). A statistically significant increase in OS was observed in other patients (that lack NPM1 mutation in combination with FLTITD WT) treated with CC-486 versus placebo (median OS 20.2 months versus 14.6 months, respectively; p=0.0119). In FIG. 10B, a significantly prolonged RFS was observed for patients harboring NPM1 mutation and FLTITD WT treated with CC-486 versus placebo (median OS 21.1 months versus 7.8 months, p=0.0572). A significant increase in RFS was observed in other patients treated with CC-486 versus placebo (median RFS 7.9 months versus 4.6 months, respectively; p=0.0006). In FIG. 10C, a trend for prolonged OS was observed comparing patients harboring NPM1 mutation and FLTITD mutation and treated with CC-486 versus placebo (median OS 49.3 months versus 11.5 months, respectively; p=0.0488). A statistically significant increase in OS was observed in other patients treated with CC-486 versus placebo (median OS 24.7 months versus 14.9 months, respectively; p=0.0080). In FIG. 10D, a significantly prolonged RFS was observed for patients harboring NPM1 mutation and FLTITD mutation treated with CC-486 versus placebo (median RFS 10.2 months versus 4.6 months, respectively; p=0.0368). A significant increase in RFS was observed in other patients treated with CC-486 versus placebo (median RFS 10.2 months versus 4.1 months, respectively; p=0.0008). In FIG. 10E, OS was not significantly different in patients harboring NPM1 WT and FLTITD mutation and treated with CC-486 versus placebo (median OS 9.2 months versus 9.7 months, respectively; p=0.9207). A statistically significant increase in OS was observed in other patients treated with CC-486 versus placebo (median OS 25.0 months versus 14.9 months, respectively; p=0.0020). In FIG. 10F, RFS was not significantly different for patients harboring NPM1 WT and FLTITD mutation treated with CC-486 vs placebo (median OS 4.5 months versus 4.0 months, respectively; p=0.9446). A significant increase in RFS was observed in other patients treated with CC-486 versus placebo (median RFS 10.2 months versus 4.9 months, respectively; p<0.0001). Figures were generated using Kaplan-Meier analyses and p-values were calculated using log-rank test.

FIG. 11A to FIG. 11D represent NPM1 in combination with FLTITD mutation status at diagnosis and associated hazard ratios for OS and RFS in patients treated with CC-486 or placebo. In FIG. 11A, OS hazard ratio for NPM1 mutation FLTITD WT, NPM1 mutation FLTITD mutation, and NPM1 WT FLTITD mutation (versus patients that lack the reported NPM1 mutation status in combination with FLTITD mutation status) in CC-486 treated patients was visualized using a forest plot (left) and reported in the table (right) along with 95% confidence interval [CI] and associated p-value. In FIG. 11B, OS hazard ratio for NPM1 mutation FLTITD WT, NPM1 mutation FLTITD mutation, and NPM1 WT FLTITD mutation (versus patients that lack the reported NPM1 mutation status in combination with FLTITD mutation status) in placebo treated patients was visualized using a forest plot (left) and reported in the table (right) along with 95% confidence interval (CI) and associated p-value. In FIG. 11C, RFS hazard ratio for NPM1 mutation FLTITD WT, NPM1 mutation FLTITD mutation, and NPM1 WT FLTITD mutation (versus patients that lack the reported NPM1 mutation status in combination with FLTITD mutation status) in CC-486 treated patients was visualized using a forest plot (left) and reported in the table (right) along with 95% confidence interval (CI) and associated p-value. In FIG. 11D, RFS hazard ratio for NPM1 mutation FLTITD WT, NPM1 mutation FLTITD mutation, and NPM1 WT FLTITD mutation (versus patients that lack the reported NPM1 mutation status in combination with FLTITD mutation status) in placebo treated patients was visualized using a forest plot (left) and reported in the table (right) along with 95% confidence interval (CI) and associated p-value. A hazard ratio below 1.0 favors NPM1 and FLTITD mutation status and a hazard ratio above 1.0 favors wild type (patients lacking the reported NPM1 mutations status in combination with FLTITD mutation status). In FIG. 11A, OS hazard ratio for CC-486 was 0.54 (95% CI 0.36 to 0.82), which favored NPM1 mutation FLTITD WT versus wild type, and this was a statistically significant result (p=0.037). No statistical significance was observed for OS hazard ratio for CC-486 was in patients with NPM1 mutation FLTITD mutation versus wild type. OS hazard ratio for CC-486 was 2.39 (95% CI 1.17 to 4.88), which favored wild type versus NPM1 WT FLTITD mutation, and this was a statistically significant result (p=0.0168). In FIG. 111B, OS hazard ratio for placebo was 0.66 (95% CI 0.45 to 0.97), which favored NPM1 mutation FLTITD WT versus wild type, and this was a statistically significant result (p=0.0334). No statistical significance was observed for OS hazard ratio for placebo in patients with NPM1 mutation FLTITD mutation or NPM1 WT FLTITD WT versus WT. In FIG. 11C, RFS hazard ratio for CC-486 was 0.54 (95% CI 0.37 to 0.80), which favored NPM1 mutation FLTITD WT versus wild type, and this was a statistically significant result (p=0.0018). RFS hazard ratio for CC-486 was 0.33 (95% CI 0.12 to 0.90, which favored NPM1 mutation FLTITD mutation versus wild type, and this was a statistically significant result (p=0.0297). No statistical significance was observed for CC-486 hazard ratio in patients harboring NPM1 WT FLTITD mutation versus wild type. In FIG. 11D, RFS hazard ratio for placebo was 0.60 (95% CI 0.41 to 0.86), which favored NPM1 mutation FLTITD WT versus wild type, and this was a statistically significant result (p=0.0064). No statistical significance was observed for RS hazard ratio for placebo in patients harboring NPM1 mutation FLTITD mutation or NPM1 WT FLTITD mutation versus WT. Cox regression analyses were performed to evaluate the association of gene mutation status and treatment arm with OS and RFS.

FIG. 12A and FIG. 12B represent for NPM1 in combination with FLTITD mutation status at diagnosis and associated hazard ratios with clinical response in patients treated with CC-486 versus placebo. In FIG. 12A, OS hazard for patients with NPM1 mutation FLTITD WT, NPM1 mutation FLTITD mutation, and NPM1 WT FLTITD mutation in CC-486 versus placebo was visualized using a forest plot (left) and reported in the table (right) along with 95% CI and associated p-value. In FIG. 12B, RFS hazard ratio for patients with NPM1 mutation FLTITD WT, NPM1 mutation FLTITD mutation, and NPM1 WT FLTITD mutation in CC-486 versus placebo was visualized using a forest plot (left) and reported in the table (right) along with 95% CI and associated p-value. A hazard ratio below 1.0 favors CC-486 and a hazard ratio above 1.0 favors placebo. In FIG. 12A, the OS hazard ratio for NPM1 mutated FLTITD WT was 0.63 (95% CI 0.38 to 1.06), which favored CC-486 treatment versus placebo, with a trend for significance (p=0.0827). The OS hazard ratio for NPM1 mutated FLTITD mutation was 0.36 (95% CI 0.13 to 1.03), which favored CC-486 treatment versus placebo, with a trend for significance (p=0.0579). No statistical significance was observed for OS hazard ratio for patients harboring NPM1 WT FLITD mutations in CC-486 versus placebo. In FIG. 12B, the RFS hazard ratio for NPM1 mutated FLTITD WT was 0.63 (95% CI 0.39 to 1.02), which favored CC-486 treatment versus placebo, with a trend for significance (p=0.0601). The RFS hazard ratio for NPM1 mutation and FLTITD mutation was 0.32 (95% CI 0.10 to 0.99), which favored CC-486 treatment versus placebo, and was statistically significant (p=0.0477). No statistical significance was observed for RFS hazard ratio for patients harboring NPM1 WT FLITD mutation in CC-486 versus placebo. Cox regression analyses were performed to evaluate the association of gene mutation status and treatment arm with OS and RFS.

FIG. 13A to FIG. 13D represent NPM1 and FLTITD and/or FLT3-TKD mutation status at diagnosis and associations with clinical response in patients treated with CC-486 versus placebo. FIG. 13A and FIG. 13B show OS for patients harboring NPM1 mutation FLTITD/FLT3-TKD wild type versus NPM1 mutation FLTITD/FLT3-TKD mutation or FLTITD/FLT3-TKD mutation NPM1 mutation versus FLTITD/FLT3-TKD mutation NPM1 WT and treated with either placebo or CC-486. FIG. 13C and FIG. 13D show RFS for patients harboring NPM1 mutation FLTITD/FLT3-TKD wild type versus NPM1 mutation FLTITD/FLT3-TKD mutation or FLTITD-FLT3-TKD mutation NPM1 mutation versus FLTITD/FLT3-TKD mutation NPM1 WT and treated with either placebo or CC-486. In FIG. 13A, a trend for prolonged OS was observed in patients harboring NPM1 mutation FLTITD/FLT3-TKD WT and treated with CC-486 vs placebo (median OS 47.1 months versus 20.2 months, respectively; p=0.0763). A trend for prolonged OS was observed in patients harboring NPM1 mutation FLTITD/FLT3-TKD mutation and treated with CC-486 versus placebo (median OS 46.1 months versus 13.4 months, respectively; p=0.0827). In FIG. 13B, no statistical significance in prolonged OS was observed for patients harboring FLTITD/FLT3-TKD mutation NPM1 WT and treated with CC-486 vs placebo (median OS 11.8 months versus 8.7 months, respectively; p=0.4105). A trend for prolonged OS was observed for patients harboring FLTITD/FLT3-TKD mutation NPM1 mutation and treated with CC-486 vs placebo (median OS 46.1 months versus 13.4 months, p=0.0827). In FIG. 13C, a trend for prolonged RFS was observed for patients harboring NPM1 mutation FLTITD/FLT3-TKD WT and treated with CC-486 versus placebo (median RFS 18.4 months versus 7.7 months, p=0.0827). A statistically significant increase in RFS was observed in patients harboring NPM1 mutation FLTITD/FLT3-TKD mutation and treated with CC-486 vs placebo (median RFS 46.1 months versus 4.9 months, respectively; p=0.0278). In FIG. 13D, no statistical significance in prolonged RFS was observed for patients harboring FLTITD/FLT3-TKD mutation NPM1 WT and treated with CC-486 vs placebo. A significant increase in RFS was observed for patients harboring FLTITD/FLT3-TKD mutation NPM1 mutation and treated with CC-486 vs placebo (median RFS 46.1 months versus 4.9 months, p=0.0278). Figures were generated using Kaplan-Meier analyses and p-values were calculated using log-rank test.

FIG. 14A and FIG. 14B represent gene mutation frequency and mutational landscape in patients (median age of 68 years) who achieved complete response CR or CRi, post-induction chemotherapy/consolidation (postIC/C). FIG. 14C shows the variant allele frequencies (VAFs) of gene mutations included in the analysis. In FIG. 14A, the most frequently mutated gene was DNMT3A, followed by TP53, IDH2, TET2, SRSF2, IDH1, and ASXL1. In FIG. 14B, the mutational landscape is presented according to functional category and separated by treatment arm. The most frequent mutations occurred in the DNA methylation category. In FIG. 14C, an increased prevalence of VAFs in genes (some pts >50% VAF in DNMT3A, TET2, ASXL1) typically classified as clonal hematopoiesis of indeterminate potential (CHIP; i.e., age-related) mutations was observed in the bone marrow, indicative that in some patients these mutations may be widely present in bone marrow cellular population (in both somatic and non-somatic hematopoietic cells). Increased prevalence of VAFs (>50% VAF) in genes such as IDH1, IDH2, and SRSF2 was also observed. Gene mutations were identified by targeted next-generation sequencing (ArcherDX VariantPlex Core Myeloid panel; 37 genes) of DNA isolated from bone marrow mononuclear cells (BMMCs).

FIG. 15 represents gene mutation status (postIC/C, in CR or CRi) and associated hazard ratios with clinical response in patients treated with CC-486 versus placebo, visualized using a forest plot (left) and reported in the table (right) along with 95% CI and associated p-value. A hazard ratio below 1.0 favors CC-486 and a hazard ratio above 1.0 favors placebo. The RFS hazard ratio for DNMT3A was 0.23 (95% CI 0.16 to 0.47), which favored CC-486 treatment versus placebo, and was statistically significant (p<0.0001). The RFS hazard ratio for TP53 was 0.53 (95% CI 0.28 to 1.02), which favored CC-486 treatment versus placebo, with a trend for significance (p=0.0587). The RFS hazard ratio for SRSF2 was 0.50 (95% CI 0.23 to 1.10), which favored CC-486 treatment versus placebo, with a trend for significance (p=0.0854). No statistical significance was observed for RFS hazard ratios for patients harboring mutations in IDH2, TET2, IDH1, or ASXL1. Figures were generated using Kaplan-Meier analyses and p-values were calculated using log-rank test.

FIG. 16A to FIG. 16D represent karyotypes (normal, abnormal, or complex) and associations with clinical response in patients that achieved CR or CRi, postIC/C. FIG. 16A and FIG. 16B show OS for patients with a normal karyotype (no abnormalities), abnormal karyotype (1-3 abnormalities), or complex karyotype (≥4 abnormalities) treated with either CC-486 or placebo. FIG. 16C and FIG. 16D show RFS for patients with a normal karyotype (no abnormalities), abnormal karyotype (1-3 abnormalities), or complex karyotype (≥4 abnormalities) treated with either CC-486 or placebo. In FIG. 16A to FIG. 16D, statistically significant increases in OS and RFS were observed for patients with a normal karyotype versus complex karyotype and patients treated with CC-486 or placebo (median OS CC-486 25.10 months versus 6.932 months, p=0.0003; median OS placebo 14.95 months versus 3.121 months, p<0.0001; median RFS CC-486 10.97 months versus 1.906 months, p<0.0001; median RFS placebo 4.895 months versus 1.150 months, p<0.0001). Other statistically significant differences in median OS or median RFS were observed for patients with normal versus abnormal karyotype and treated with CC-486. Statistically significant differences in median OS or median RFS were also observed for patients with abnormal versus complex karyotype and treated with placebo. Figures were generated using Kaplan-Meier analyses and p-values were calculated using log-rank test.

FIG. 17A to FIG. 17D represent karyotypes (normal or abnormal) and associations with clinical response in patients that achieved CR or CRi, postIC/C. FIG. 17A and FIG. 17B show OS and RFS for patients with a normal karyotype treated with either CC-486 or placebo. FIG. 17C and FIG. 17D show OS and RFS for patients with an abnormal karyotype (>1 abnormality) treated with either CC-486 or placebo. In patients with a normal karyotype, statistically significant increases in OS and RFS were observed in patients treated with CC-486 versus placebo (median OS 25.1 months versus 14.95 months, p=0.004; median RFS 10.97 months versus 4.895 months, p<0.0001). In patients with an abnormal karyotype, no statistically significant differences in OS or RFS were observed between CC-486 and placebo. Figures were generated using Kaplan-Meier analyses and p-values were calculated using log-rank test.

FIG. 18A to FIG. 18D represent gene mutations (DNMT3A or p53) and associations with clinical response in patients that achieved CR or CRi, postIC/C. FIG. 18A and FIG. 18B show OS and RFS for patients with DNMT3A mutation or WT and treated with either CC-486 or placebo. FIG. 18C and FIG. 18D show OS and RFS for patients with p53 mutation or WT and treated with either CC-486 or placebo. In FIG. 18A and FIG. 18B, statistically significant increases in OS and RFS were observed for patients with DNMT3A mutation and treated with CC-486 versus placebo (median OS 24.8 months versus 13.0 months, p=0.0003; median RFS 18.4 months versus 4.7 months, p<0.0001). No differences in OS or RFS were observed for patients with WT DNMT3A and treated with CC-486 versus placebo. In FIG. 18C, no statistically significant differences in OS were observed between CC-486 and placebo in patients with p53 mutation. However, in FIG. 18D, a trend for prolonged RFS was observed for patients with p53 mutation and treated with CC-486 versus placebo (median RFS 7.4 months versus 3.5 months, p=0.0579). For patients with WT p53, increases in OS and RFS were observed comparing CC-486 versus placebo. Figures were generated using Kaplan-Meier analyses and p-values were calculated using log-rank test.

FIG. 19A to FIG. 19D represent mutations in functional categories and associated hazard ratios for RFS. RFS hazard ratios for mutations in functional categories are reported for patients treated with CC-486 (FIG. 19A) or placebo (FIG. 19B). A hazard ratio below 1.0 favors mutations in the reported functional category and a hazard ratio above 1.0 does not favor mutations in the reported functional category. RFS hazard ratios for mutations in functional categories for patients treated with CC-486 versus placebo were visualized using a forest plot (FIG. 19C) and reported in the table (FIG. 19D) along with 95% confidence interval (CI) and associated p-value. A hazard ratio below 1.0 favors CC-486 and a hazard ratio above 1.0 favors placebo. In FIG. 19A, RFS hazard ratio favoring DNA methylation category was observed in patients treated with CC-486, with a trend for significance. The RFS hazard ratio for patients with mutations in the RAS pathway treated with CC-486 was greater than 1.0 (not in favor of mutations in the pathway), and this was statistically significant. No other statistically significant RFS hazard ratios were observed for the other functional categories. In FIG. 19B, RFS hazard ratio for patients with mutations in DNA transcription or Receptors/Kinases/Signaling categories treated with placebo was greater than 1.0, and there was a trend for significance. The RFS hazard ratio for patients with mutations in the RAS pathway treated with placebo was greater than 1.0 (not in favor of mutations in the pathway), and this was statistically significant. No other statistically significant RFS hazard ratios were observed for the other functional categories. In FIG. 19C and FIG. 19D, RFS hazard ratios for patients with mutations in DNA methylation and transcription were observed favoring CC-486 treatment versus placebo (hazard ratios less than 1.0), and both were statistically significant. Cox regression analyses were performed to evaluate the association of gene mutation status and treatment arm with OS and RFS.

FIG. 20A and FIG. 20B represent statistical associations of co-occurring gene mutations in patients that achieved CR or CRi, postIC/C. The commutation frequencies are listed in the Table (FIG. 20A). FIG. 20B is a heat map showing the odds ratio for all possible pairwise comparisons between individual gene mutations, karyotypes, chromosomal abnormalities, and fusion genes. The list of genes that were included in the co-mutation analysis in FIG. 20B include: ASXL1, BCOR, BRAF, CALR, CBL, CEBPA, DDX41, DNMT3A, ETV6, EZH2, FLT3, FLTITD, GATA2, IDH1, IDH2, JAK2, KIT, KRAS, MPL, NPM1, NRAS, PHF6, RUNX1, SETBP1, SF3B1, SRSF2, STAG2, TET2, TP53, U2AF1, WT1, and ZRSR2. In FIG. 20A and FIG. 20B, the top three associations in the data set were IDH2 and SRSF2, DNMT3A and TET2, and DNMT3A and IDH2. 2×2 contingency tables were generated to test putative associations using Fisher's exact tests and adjusted to control for multiple hypothesis testing.

FIG. 21A to FIG. 21D represent single gene mutations (SRSF2 or IDH2) or co-occurring mutations in SRSF2/IDH2 and associations with clinical response in patients that achieved CR or CRi, postIC/C. FIG. 21A and FIG. 21B show the clinical associations (RFS) in patients with mutations in SRSF2 alone or SRSF2 WT, or IDH2 alone or IDH2 WT and treated with CC-486 versus placebo. RFS curves for co-occurrence of SRS2 and IDH2 versus SRSF/IDH2 WT (other) are demonstrated with CC-486 treatment (FIG. 21C) or placebo (FIG. 21D). A trend for improved RFS was observed for patients with mutations in SRSF2 alone and treated with CC-486 versus placebo (FIG. 21A, p=0.0809). A trend for decreased RFS was observed for patients with SRSF2/IDH2 co-mutation and treated with placebo (FIG. 21D, p=0.0617). No other significant differences in RFS were observed in single gene mutation queries or co-occurring mutations. Figures were generated using Kaplan-Meier analyses and p-values were calculated using log-rank test.

The embodiments provided herein are not to be limited in scope by the specific embodiments provided in the Example, which are intended as illustrations of a few aspects of the provided embodiments and any embodiments that are functionally equivalent are encompassed by the present disclosure. Indeed, various modifications of the embodiments provided herein are in addition to those shown and described herein will become apparent to those skilled in the art and are intended to fall within the scope of the appended claims.

All disclosures (e.g., patents, publications, and web pages) referenced throughout this specification are incorporated by reference in their entireties.

1. A method of treating a cancer or a disorder related to abnormal cell proliferation in a human subject, comprising administering to the human subject a therapeutically effective amount of a hypomethylating agent, and wherein the cancer or the disorder is characterized by:
(i) presence of a nucleophosmin (NPM1) mutation;
(ii) presence of a NPM1 mutation and a FMS-like tyrosine kinase-3 (FLT3) mutation;
(iii) presence of a DNA methyltransferase 3A (DNMT3A) mutation;
(iv) presence of a tumor protein p53 (TP53) mutation; or
(v) presence of an isocitrate dehydrogenase 2 (IDH2) mutation and a serine-and-arginine-rich-splicing-factor 2 (SRSF2) mutation.

2. A method of treating a cancer or a disorder related to abnormal cell proliferation in a human subject, comprising:
(1) identifying the cancer or the disorder in the human subject to be characterized by:
(i) presence of a nucleophosmin (NPM1) mutation;
(ii) presence of a NPM1 mutation and a FMS-like tyrosine kinase-3 (FLT3) mutation;
(iii) presence of a DNA (cytosine-5)-methyltransferase 3A (DNMT3A) mutation;
(iv) presence of a tumor protein p53 (TP53) mutation; or
(v) presence of an isocitrate dehydrogenase 2 (IDH2) mutation and a serine-and-arginine-rich-splicing-factor 2 (SRSF2) mutation; and
(2) administering to the human subject a therapeutically effective amount of a hypomethylating agent.

3. The method of claim 1, wherein the cancer or the disorder is characterized by the presence of a NPM1 mutation.

4. The method of claim 1, wherein the cancer or the disorder is characterized by the presence of a NPM1 mutation and a FLT3 mutation.

5. The method of claim 4, wherein the FLT3 mutation is a FMS-like tyrosine kinase-3 internal tandem duplication (FLT3-ITD) or FMS-like tyrosine kinase-3 tyrosine kinase domain (FLT3-TKD) mutation.

6. The method of claim 1, wherein the cancer or the disorder is characterized by the presence of a DNMT3A mutation.

7. The method of claim 1, wherein the cancer or the disorder is characterized by the presence of a TP53 mutation.

8. The method of claim 1, wherein the cancer or the disorder is characterized by the presence of an IDH2 mutation and a SRSF2 mutation.

9. The method of claim 1, wherein the cancer or the disorder is further characterized by absence of a RAS mutation.

10. The method of claim 1, wherein the human subject tests negative for minimal residual disease (MRD).

11. The method of claim 10, wherein the cancer or the disorder is characterized by the presence of a NPM1 mutation, and the human subject tests negative for MRD.

12. A method of preventing a clonal hematopoiesis of indeterminate potential (CHIP) disease in a human subject from progressing into a cancer or a disorder related to abnormal cell proliferation, comprising:
(1) identifying the CHIP disease in the human subject to be characterized by:
(i) presence of a DNA (cytosine-5)-methyltransferase 3A (DNMT3A) mutation;
(ii) presence of an additional sex comb-like 1 (ASXL1) mutation; or
(iii) presence of ten-eleven translocation-2 (TET2) mutation; and
(2) administering to the human subject a therapeutically effective amount of a hypomethylating agent.

13. A method of treating a cancer or a disorder related to abnormal cell proliferation in a human subject with a hypomethylating agent, comprising:
(a) identifying the human subject having the cancer or the disorder that may be responsive to the treatment comprising the hypomethylating agent, comprising:
i. detecting the presence of one or more gene mutations in a sample obtained from the patient; and
ii. identifying the human subject as being likely to be responsive to the treatment comprising the hypomethylating agent if one or more gene mutations are detected; and
(b) administering to the human subject a therapeutically effective amount of the hypomethylating agent if the human subject is identified as being likely to be responsive to the treatment;
wherein the one or more gene mutations are detected in NPM1, FLT3, DNMT3A, TP53, IDH2 and/or SRSF2.

14-22. (canceled)

23. The method of claim 1, wherein the hypomethylating agent is administered as the only therapeutic agent or therapy.

24. The method of claim 1, further comprising administering at least one additional therapeutic agent or therapy.

25. The method of claim 24, wherein the at least one additional therapeutic agent or therapy is an agent that modulates NPM1 pathway or targets nucleophosmin, a FLT3 inhibitor, an agent or therapy that restore the wild-type DNMT3A function, a PI3K/Akt/mTOR pathway inhibitor, a TP53 inhibitor, an agent that targets spliceosome or its downstream target(s), an IDH2 inhibitor, or a RAS pathway inhibitor.

26. The method of claim 25, wherein the at least one additional therapeutic agent or therapy is SGC0946, pinometostat, MI-503, midostaurin, lestaurtinib, sunitinib, sorafenib, quizartinib, crenolanib, pexidartinib, gilteritinib, a small molecule compound or a therapy that utilizes genetic method CRISPR, rapamycin or an analog thereof (also termed rapalog), everolimus, idelalisib, ipatasertib, FR901464, herboxidiene, pladienolide, meayamycin, E7107, spliceostatin A, ivosidenib, enasidenib, 2-methyl-1-[(4-[6-(trifluoromethyl)pyridin-2-yl]-6-{[2-(trifluoromethyl)pyridin-4-yl]amino}-1,3,5-triazin-2-yl)amino]propan-2-ol, captopril, imidapril, zofenopril, candesartan, delapril, telmisartan, aliskiren, moexipril, enalapril, valsartan, fosinopril, irbesartan, perindopril, quinapril, ramipril, eprosartan, olmesartan, trandolapril, losartan, azilsartan, lisinopril, spirapril, benazepril, cilazapril, vemurafenib, dabrafenib, trametinib, selumetinib, binimetinib, PD-325901, cobimetinib, CI-1040, PD035901, LY3214996, LTT462, or BVD-523.

27-50. (canceled)

51. The method of claim 25, wherein: (i) the cancer or the disorder is characterized by the presence of a NPM1 mutation, and the at least one additional therapeutic agent or therapy is an agent that modulates NPM1 pathway or targets nucleophosmin or a TP53 inhibitor;
(ii) wherein the cancer or the disorder is characterized by the presence of a NPM1 mutation and a FLT3 mutation, and the at least one additional therapeutic agent or therapy is a FLT3 inhibitor;
(iii) wherein the cancer or the disorder is characterized by the presence of a DNMT3A mutation, and the at least one additional therapeutic agent or therapy is an agent that modulates NPM1 pathway or targets nucleophosmin, an agent or therapy that restore the wild-type DNMT3A function, or a PI3K/Akt/mTOR pathway inhibitor;
(iv) wherein the cancer or the disorder is characterized by the presence of a TP53 mutation, and the at least one additional therapeutic agent or therapy is a TP53 inhibitor;
(v) wherein the cancer or the disorder is characterized by the presence of an IDH2 mutation and a SRSF2 mutation, and the at least one additional therapeutic agent or therapy is an agent that targets spliceosome or its downstream target(s) or an IDH2 inhibitor; or
(vi) wherein the cancer or the disorder is further characterized by the absence of a RAS mutation, and the at least one additional therapeutic agent or therapy is a RAS pathway inhibitor.

52-77. (canceled)

78. The method of claim 1, wherein the hypomethylating agent is 5-azacytidine.

79. The method of claim 78, wherein the 5-azacytidine is in orally administered as a composition that is non-enteric-coated.

80. The method of claim 1, wherein the hypomethylating agent is decitabine, guadecitabine, or zebularine.

81. The method of claim 1, wherein the cancer is a hematological cancer.

82. The method of claim 81, wherein the hematological cancer is acute myeloid leukemia (AML), acute lymphoblastic leukemia (ALL), chronic myeloid leukemia (CML), chronic lymphocytic leukemia (CLL), non-Hodgkin's lymphoma (NHL), Hodgkin's lymphoma, or multiple myeloma (MM).

83. The method of claim 82, wherein the hematological cancer is acute myeloid leukemia (AML).

84. The method of claim 83, wherein the AML is secondary to prior myelodysplastic disease (MDS) or chronic myelomonocytic leukemia (CMML).

85. The method of claim 83, wherein the AML is relapsed or refractory AML.

86. The method of claim 83, wherein the AML has intermediate-risk cytogenetic characteristics as defined according to National Comprehensive Cancer Network 2011 guidelines.

87. The method of claim 83, wherein the AML has poor-risk cytogenetic characteristics as defined according to National Comprehensive Cancer Network 2011 guidelines.

88. The method of claim 1, wherein the cancer is a solid tumor.

89. The method of claim 88, wherein the solid tumor is melanoma, carcinoma, adenocarcinoma, chordoma, breast cancer, colorectal cancer, ovarian cancer, lung cancer, testicular cancer, renal cancer, pancreatic cancer, bone cancer, gastric cancer, head and neck cancer, or prostate cancer.

90. The method of claim 1, wherein the disorder related to abnormal cell proliferation is myelodysplastic syndromes (MDS).
